(19) United States
(12) Patent Application Publication
(10) Pub. No.: US 2004/0039175 A1
(43) Pub. Date: Feb. 26, 2004

(54) Modulation of viral gene expression by engineered zinc finger proteins

(76) Inventors: Yen Choo, Cambridge (GB); Christophe Demaison, London (GB); Michael Moore, Amersham Bucks (GB); Monika Anna Papworth, Cambs (GB); Lindsey Reynolds, Hertsfordshire (GB); Christopher Graeme Ullman, London (GB); Mark Isalan, London (GB)

Correspondence Address: COOLEY GODWARD, LLP, 3000 EL CAMINO REAL, 5 PALO ALTO SQUARE, PALO ALTO, CA, 94306, US

(21) Appl. No.: 10/276,608

(22) Filed: Nov. 7, 2002

(30) Foreign Application Priority Data
May 8, 2001 (WO) PCT/GB01/02017
Jan. 19, 2001 (GB) 0101446.3
Oct. 2, 2000 (WO) PCT/GB00/03765
May 30, 2000 (GB) 0013106.0
May 8, 2000 (GB) 0011068.4

(51) Int. Cl. C12N 9/22; C07K 16/10

(52) U.S. Cl. 530/388.35; 435/199

(57) ABSTRACT

We disclose a polypeptide capable of binding to a nucleic acid comprising a viral nucleotide sequence. Preferably, the viral nucleotide sequence comprises a viral promoter sequence, for example, an HIV promoter or a herpesvirus promoter sequence.

### FIELD OF THE INVENTION

The present invention relates to molecules. In particular, the present invention relates to molecules capable of binding to viral nucleotide sequences.

### BACKGROUND TO THE INVENTION

Many diseases are caused by viral infections. Infection of humans with Human Immunodeficiency Virus such as HIV-1 causes a dramatic decline in the numbers of white blood cells, particularly in the numbers of CD4+ T-lymphocytes. When the number of such cells becomes low enough, opportunistic infections and neoplasms occur, and the pathology may progress to Advanced Immune Deficiency Syndrome (AIDS).

Infection with Herpes Simplex Virus produces a variety of clinical syndromes, including cold sores and genital lesions, as well as neonatal herpes, herpes encephalitis, eye infections, and disseminated infections of the internal organs. Therapeutics aimed at combating HIV, HSV, and other viruses, as well as research tools for their study, are extremely important.

A zinc finger is a DNA-binding protein domain that may be used as a scaffold to design DNA-binding proteins with predetermined sequence-specificity (3, 4). The peptide motif comprises about 30 amino acids that adopt a compact DNA-binding structure on chelating a zinc ion (5). Each zinc finger module is capable of recognising 34 bp of DNA, such that arrays comprising tandemly repeated modules bind proportionally longer nucleotide sequences. The crystal structure of the Zif268 DNA-binding domain, in complex with its optimal DNA binding site, shows that the zinc finger array wraps around the DNA, with the α-helix of each finger buried in the major groove (6).

DNA-binding domains with predetermined sequence-specificity have been engineered by selection of zinc finger modules using phage display, allowing the construction of customised transcription factors using available protein engineering methods (1, 2). Phage display libraries of zinc fingers have been used to select individual zinc fingers with predetermined DNA-binding specificities (1, 2, 7-15). Two protein engineering strategies (recently reviewed in (16)) have been developed to facilitate construction of DNA-binding domains using such zinc fingers, however both methods exhibit certain limitations, and are not of general applicability.

An earlier engineering strategy (1), and a recent derivative thereof (13), involve parallel pre-selection of individual zinc fingers and subsequent combination of these modules to produce a polymeric zinc finger molecule. The implementation of this strategy is currently limited to producing proteins that only bind to DNA sequences with guanine repeated at every third base (eg. GNNGNN . . . ).

Greisman and Pabo's strategy of serial zinc finger selections (2, 17), though allowing for binding to more diverse DNA targets, appears too cumbersome for widespread application, and is a highly labour-intensive procedure. The prior art appears to describe only a few different zinc finger DNA-binding domains with non-arbitrary binding specificities, these having been produced using phage display (1, 2, 10, 15).

The present invention seeks to overcome one or more problem(s) associated with the prior art.

### SUMMARY OF THE INVENTION

According to a first aspect of the present invention, we provide a polypeptide capable of binding to a nucleic acid comprising a viral nucleotide sequence. Other aspects of the invention, and preferred embodiments, are set out in the independent claims as well as in the description.

### BRIEF DESCRIPTION OF THE FIGURES

FIG. 1. Overview of the protein engineering strategy. Step 1. Two pre-made zinc finger phage-display libraries, Lib12 and Lib23, contain randomised DNA-binding amino acid positions in fingers 1 and 2 (black) or fingers 2 and 3 (grey) respectively. Selections of ‘one-and-a-half’ fingers from each master library are carried out in parallel using DNA sequences in which 5 nucleotides have been fixed to a sequence of interest. Step 2. Zinc finger genes are amplified from the recovered phage using PCR and sets of ‘one-and-a-half’ fingers are paired to yield recombinant three-finger DNA-binding domains. Step 3. The recombinant DNA-binding domains are cloned back into phage and subjected to further rounds of selection, or immediately validated for binding to a composite 10 bp DNA of pre-defined sequence.

FIG. 2. Composition of the ‘bipartite’ library. (a) DNA recognition by the two zinc finger master libraries, Lib12 and Lib23. The libraries are based on the three-finger DNA-binding domain of Zif268 and the putative binding scheme is based on the crystal structure of the wild-type domain in complex with DNA (6, 22). The DNA-binding positions of each zinc finger are numbered and randomised residues in the two libraries are circled. Broken arrows denote possible DNA contacts from Lib12 to bases H′IJKLM and from Lib23 to bases MNOPQ. Solid arrows show DNA contacts from those regions of the two libraries that carry the wild-type Zif268 amino acid sequence, as observed in the crystal structure. The wild-type portion of each library target site (white boxes) determines the register of the zinc finger-DNA interactions, such that the selected portions of the two libraries can be recombined to recognise the composite site H′IJKLMNOPQ. (b) Amino acid composition of the randomised DNA-binding positions on the α-helix of each zinc finger. A subset of the 20 amino acids is included in each DNA-binding position. Note that positions 4 and 5 of F2 (LS) are specified by the codons CTG AGC, which contain the recognition site of the restriction enzyme DdeI (underlined), used as a breakpoint to recombine the products of the two libraries.

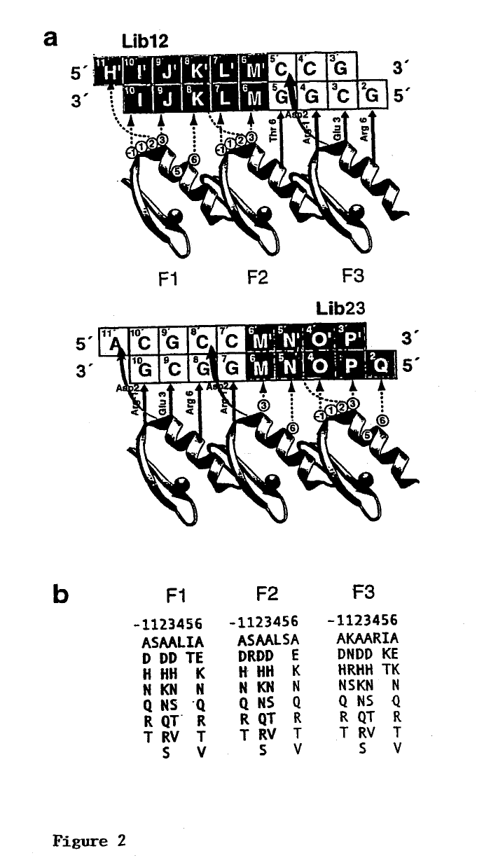

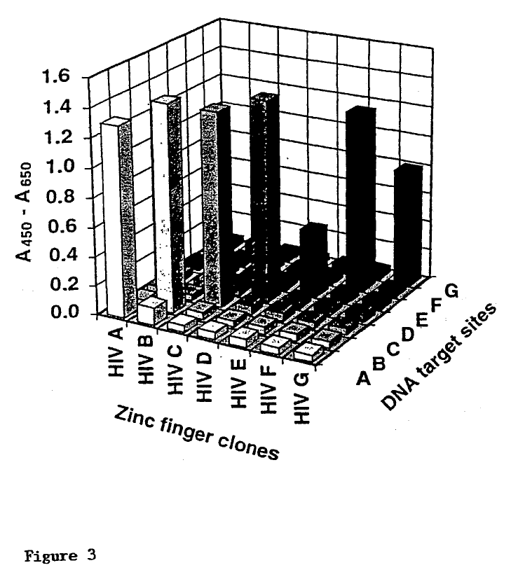

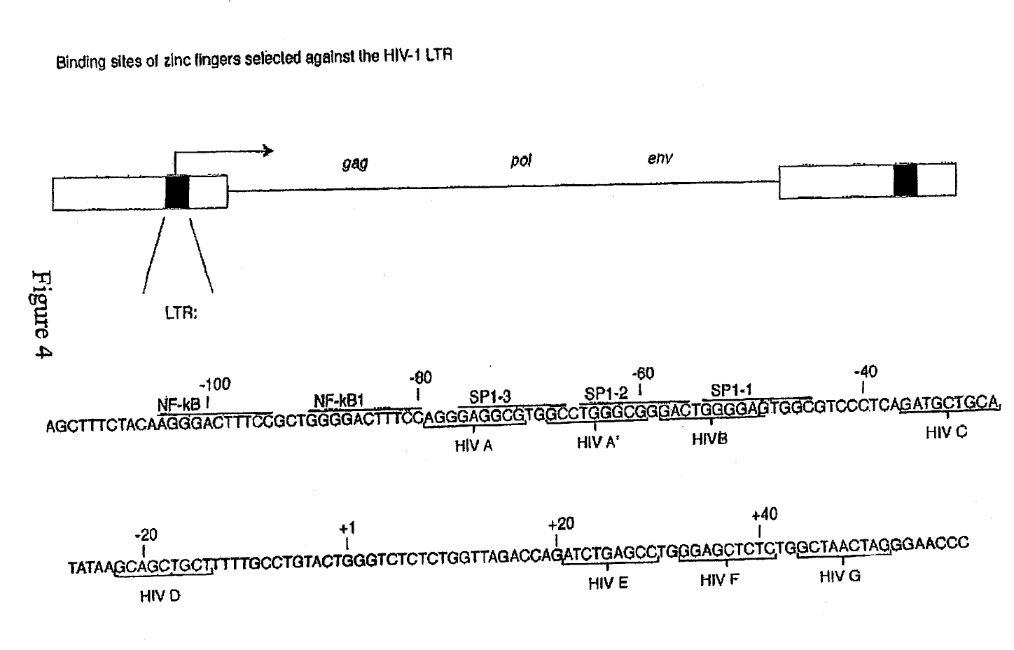

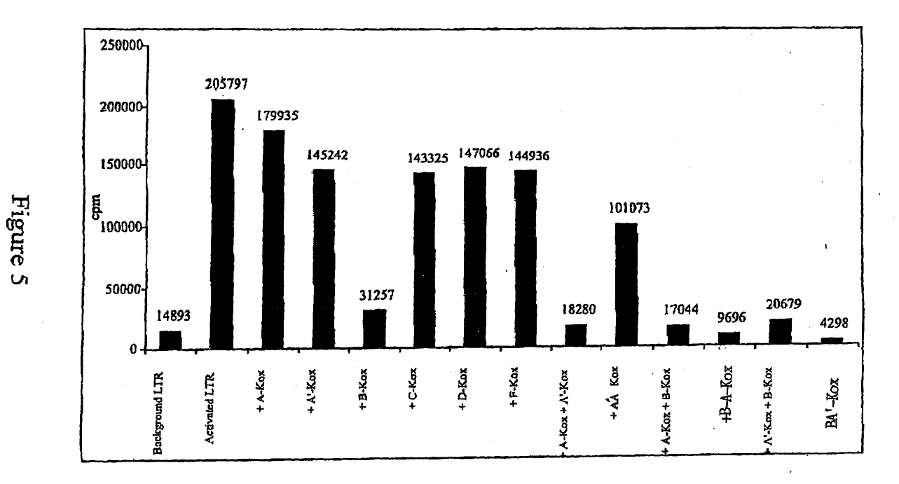

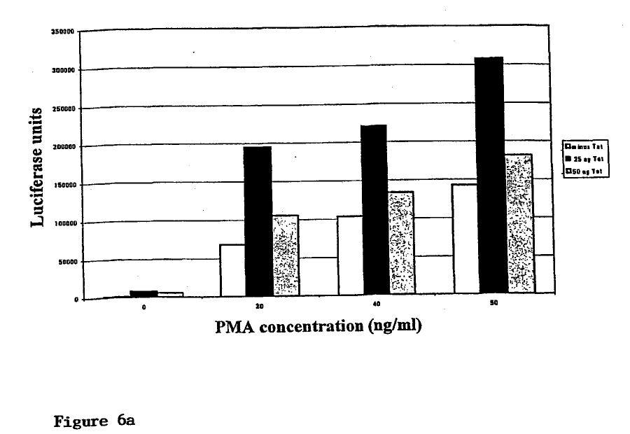

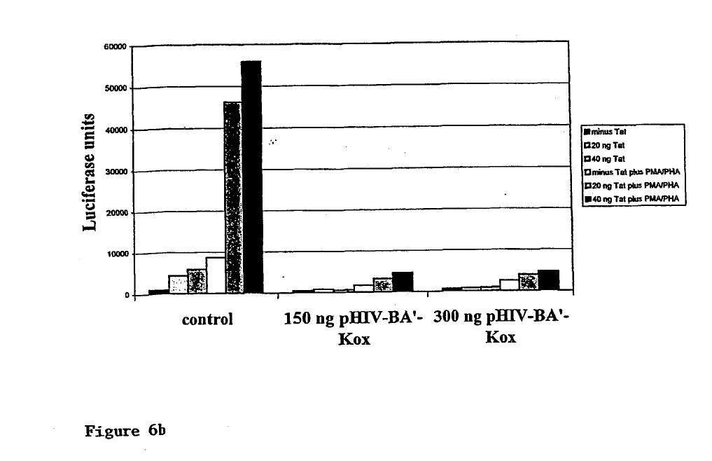

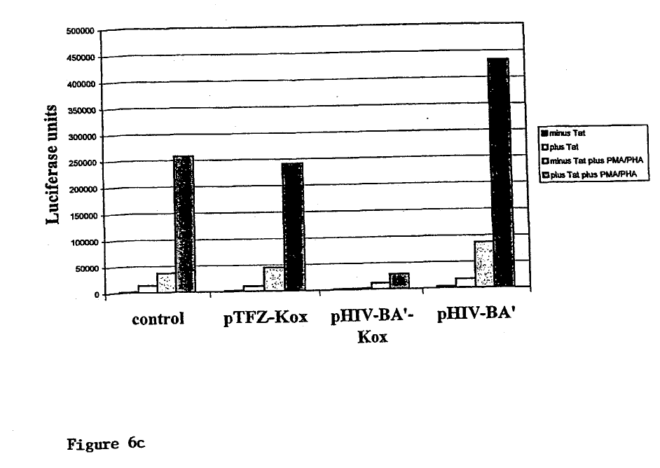

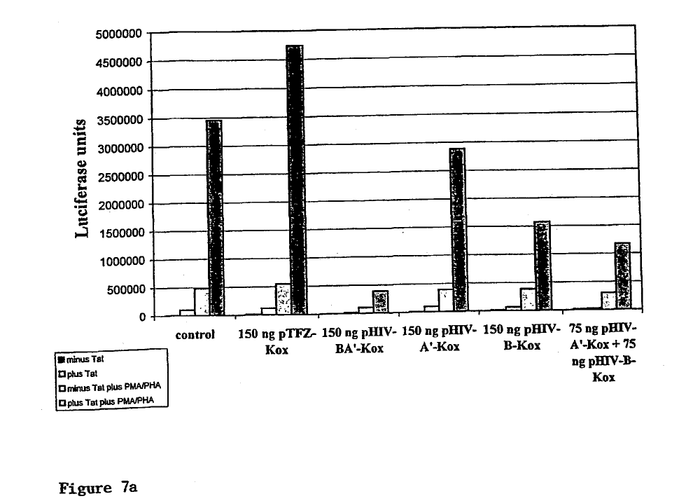

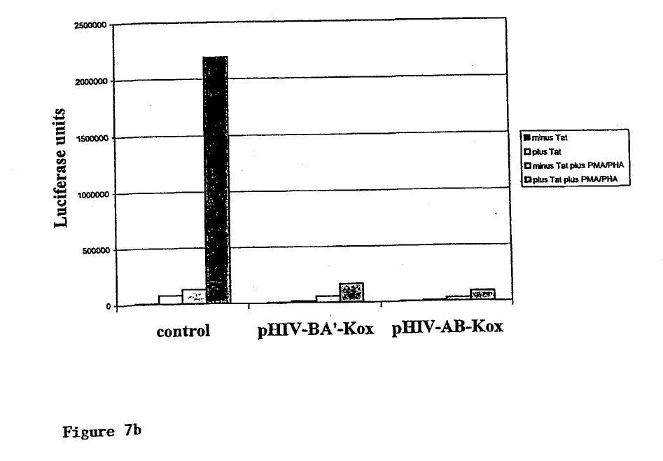

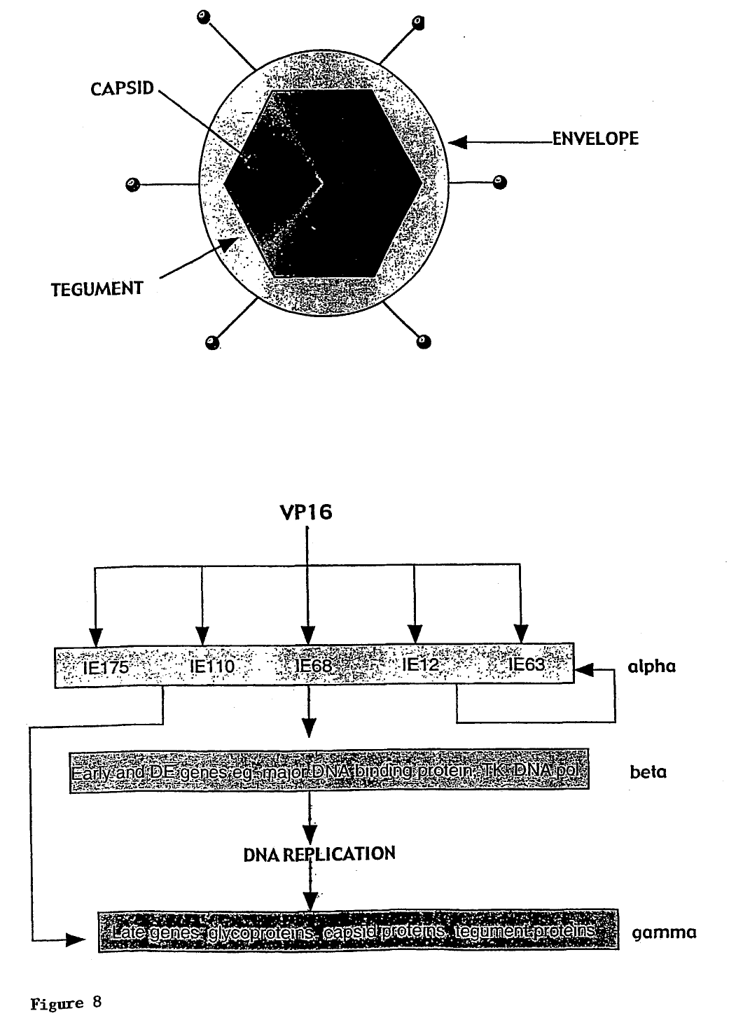

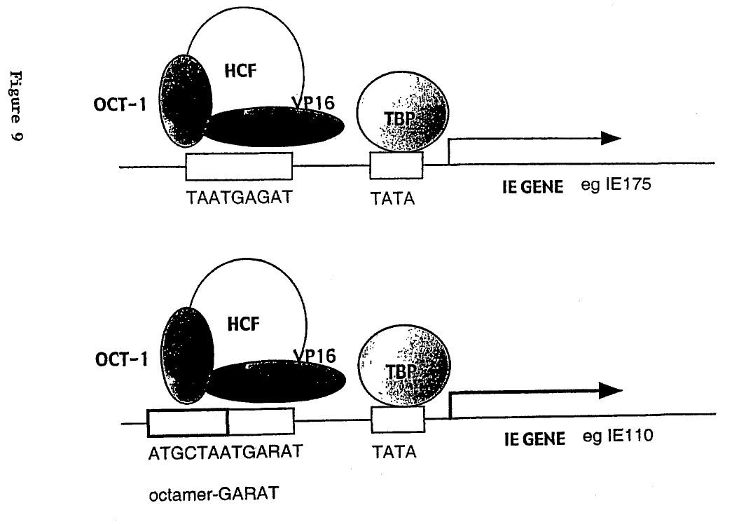

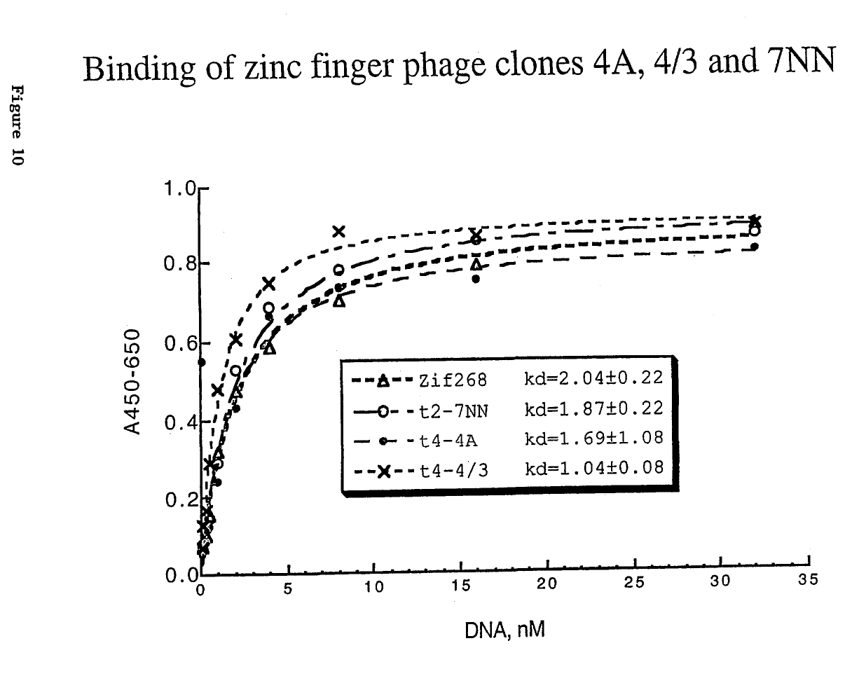

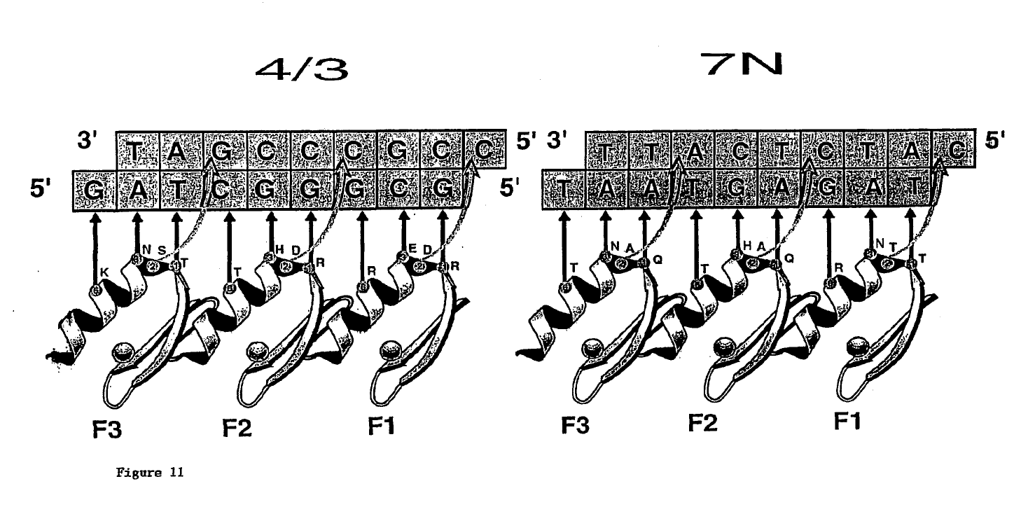

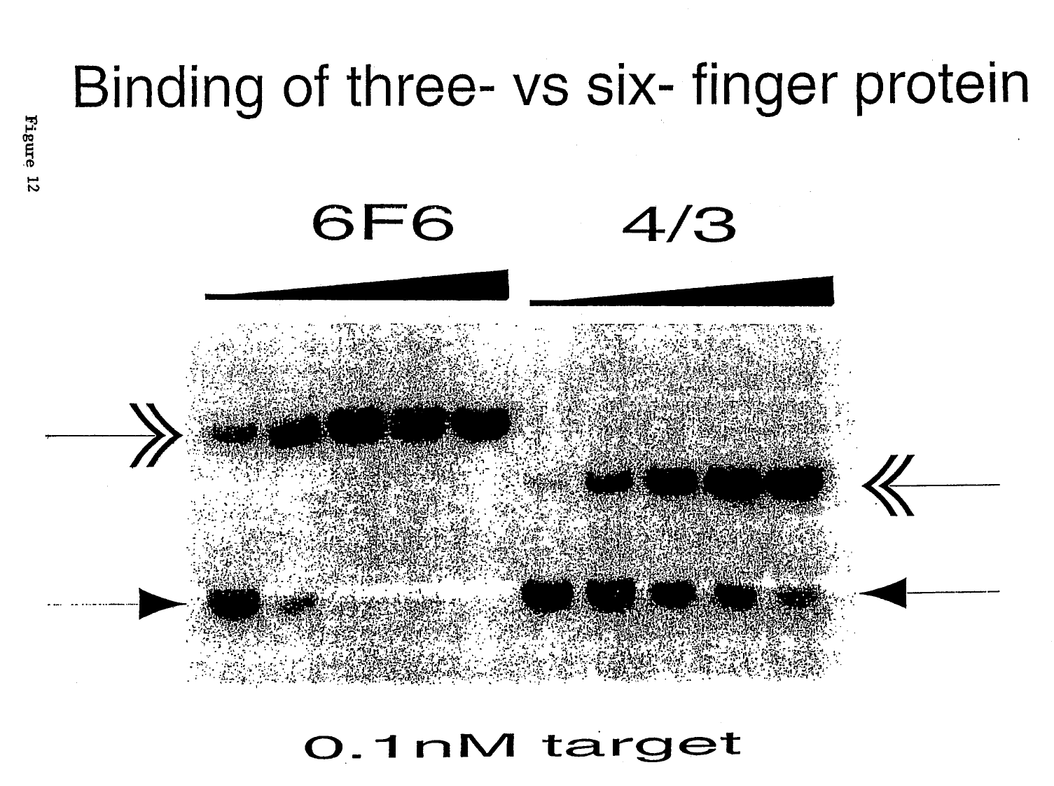

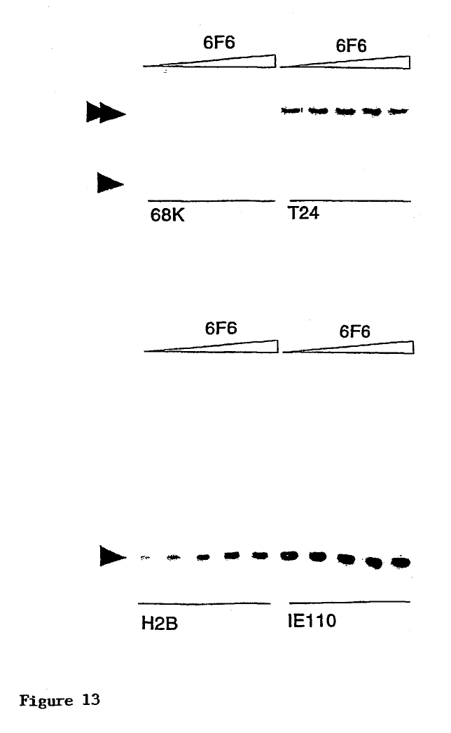

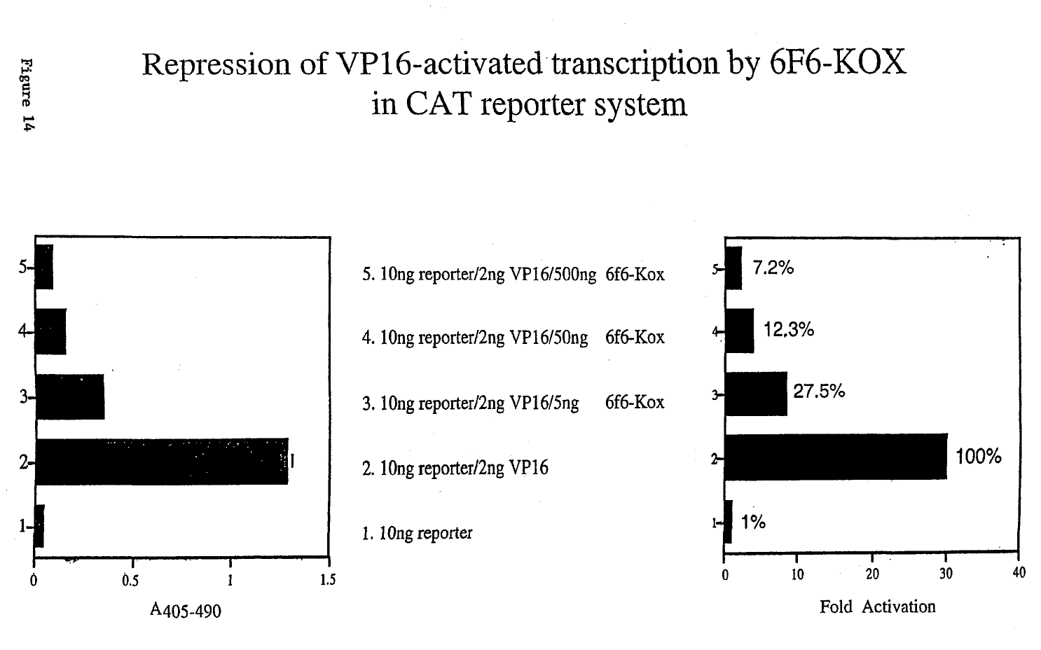

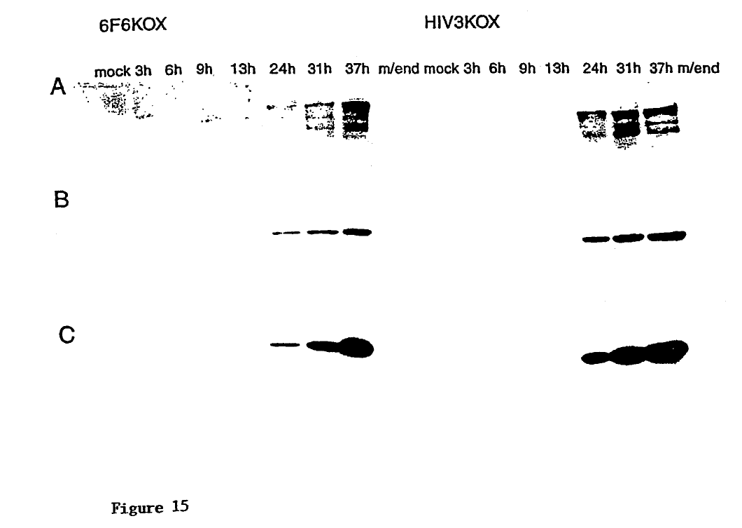

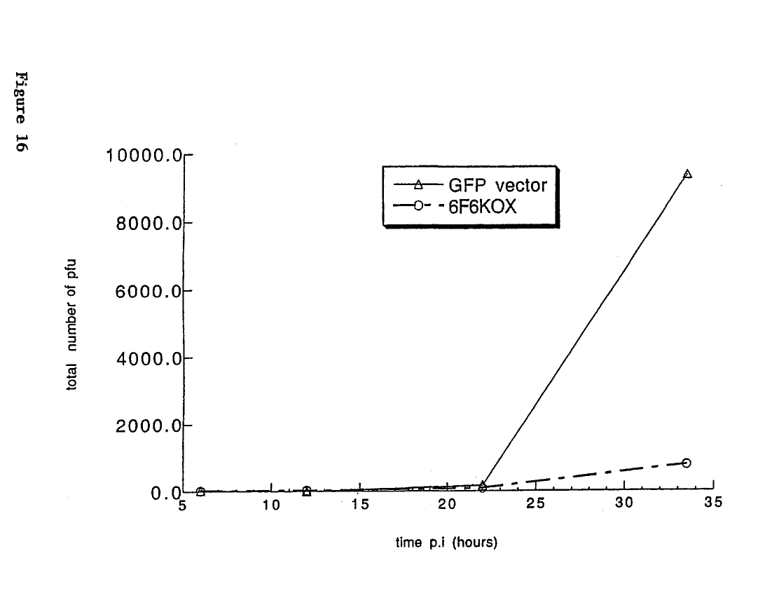

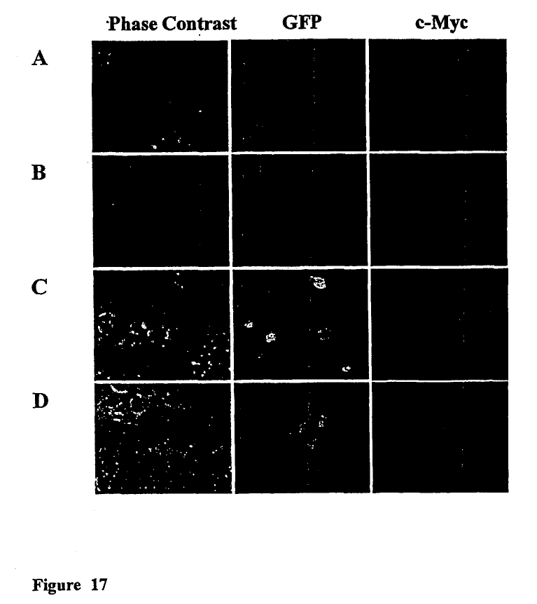

Table 1. Selection of DNA-binding domains to recognise the HIV-1 promoter. (a) Nucleotide sequences from HIV-1 of the form 3′-HIJKLMNOPQ-5′ as recognised by phage clones A-G. Bases which are predicted to be bound by amino acid residues from Lib12 and Lib23, according to the model described in FIG. 2, are shown. The position of base Q in each site is numbered relative to the transcription start site (+1) in the HIV promoter. Note that the binding site for Clone HIV-A contains 5 bases from the binding site of Zif268 (underlined); and that this clone is thus derived directly from Lib23, without the need for recombination. (b) Amino acid sequences of the helical regions from recombinant zinc finger DNA-binding domains that recognise HIV-1 sequences. The origin of the amino acids is indicated by shading Lib12 and Lib23 residues. Clone HIV-A, which is derived solely from Lib23, contains wild-type Zif23 residues (underlined). (c) Apparent K_{d} for the interaction of the customised DNA-binding domains for their cognate sequences as measured by phage ELISA.

FIG. 3. Matrix specificity assay for seven zinc finger DNA-binding domains designed to bind sequences in the HIV-1 promoter. The seven constructs and their respective binding sites are labelled A-G. Binding of zinc fingers to 0.4 pmol DNA per 50 μl well is plotted vertically from phage ELISA absorbance readings (A₄₅₀-A₆₅₀). Each clone is tested using all seven DNA sequences but strong binding is only observed to those sequences against which they had been designed.

FIG. 4. Binding sites of zinc finger DNA binding doamins selected to recognise the HIV-1 LTR. Shown is the 9 kbp HIV-1 genome encoding the gag pol env genes and the 5′ and 3′ long terminal repeats (LTR). These genes are transcribed from a single promoter in the 5′ LTR, the DNA sequence of which is shown in detail. This is the sequence as reported by Jones and Peterlin Annu. Rev. Biochem. 63:717-743 (1994). The DNA bases in the sequence are numbered relative to the transcription start site (+1). Highlighted above the sequence are the binding sites for the human transcription factors NF-kB and SPI. Highlighted below the sequence are the sites targeted by exemplary zinc finger DNA binding domains selected by the bipartite selection strategy as described herein (HIV-A, HIV-A′, HIV-B to HIV-G).

FIG. 5. Bar chart showing the expression/transcription from a LTR-CAT reporter plasmid transfected into COS7 cells measured as the CAT activity in counts per million (cpm). Shown is the activating effect of Tat on the LTR (Activated LTR′) and the repressing effect of zinc finger repressor proteins HIV-A-KOX (A-KOX), HIV-A′-KOX (A′-KOX), HIV-B-KOX (B-KOX), HIV-C-KOX (C-KOX), HIV-D-KOX (D-KOX), and HIV-F-KOX (F-KOX) on the ‘Activated LTR’. Also shown are the repressive effects combinations of three finger proteins such as A-KOX+A′-KOX, A-KOX+B-KOX, A′-KOX+B-KOX and six finger proteins such as HIV-A′A-KOX (A′A-KOX), HIV-BA-KOX (BA-KOX) and HIV-BA′-KOX (BA′-KOX) have on the ‘Activated LTR’.

FIG. 6A. Graph showing the amount of luciferase activity produced by transcription from the HIV LTR in the presence of varying concentrations of PMA and in the absence (empty bars) or presence of 25 ng of the Tat-expressing plasmid (black bars), or 50 ng of the plasmid (grey bars).

FIG. 6B. Graph showing the amount of luciferase activity produced by transcription from the HIV LTR in the absence or presence of 150 ng or 300 ng of the plasmid expressing the HIV-inhibitory peptide HIV-BA′-KOX. Experiments are carried out in the absence or presence of different amounts of the Tat-expressing plasmid, PMA and PHA, as indicated.

FIG. 6C. Graph showing the amount of luciferase activity produced by transcription from the HIV LTR in the absence or presence of the control plasmid or the plasmids expressing the peptides HIV-BA′-KOX or HIV-BA′. Experiments are carried out in the absence or presence of the Tat-expressing plasmid, PMA and PHA, as indicated.

FIG. 7A. Graph showing the amount of luciferase activity produced by transcription from the HIV LTR in the absence or presence of the control plasmid or the plasmids expressing the peptides HIV-BA′-KOX, HIV-A′-KOX, and/or HIV-B-KOX. Experiments are carried out in the absence or presence of the Tat-expressing plasmid, PMA and PHA, as indicated.

FIG. 7B. Graph showing the amount of luciferase activity produced by transcription from the HIV LTR in the absence or presence of the plasmids expressing the peptides HIV-BA′-KOX and HIV-AB-KOX. Experiments are carried out in the absence or presence of the Tat-expressing plasmid, PMA and PHA, as indicated.

FIG. 8. HSV-1 virus structure and cascade of HSV-1 gene expression FIG. 9. Mechanism of activation of HSV-1 IE genes by VP16 interaction with TAATGARAT elements. Two types of TAATGARAT sites—octa+ and octa− are shown on IE175k and IE110k promoters respectively

FIG. 10. Binding of 3-finger proteins to their target sites. Selected phage clones 4/3, 4A and 7N are used for phage ELISA experiment on serial dilutions of their binding sites. Zif 268 displayed on the phage is used as a control. The ELISA readings (at 450-650 nm) are plotted against DNA concentrations in nM

FIG. 11. Predicted amino acid to base contacts between 3-finger proteins (4/3 and 7N) and their target sites. Major contacts (amino acids at position −1, 3 and 6) are shown as solid arrows and cross-strand contacts are shown as shaded curved arrows.

FIG. 12. In vitro binding of 3- versus 6-finger proteins. The 6F6 and 4/3 proteins are expressed in the in vitro transcription/translation system and used in 5-fold dilutions in gel retardation assay with T24 DNA probe (used at 0.1 nM). Solid single-headed arrows mark the position of free unbound probe while double-headed arrows show the position of protein-DNA complexes

FIG. 13. In vitro binding of 6F6-KOX to IE175k target sites and related sequences. The 6F6 protein is expressed in the in vitro transcription/translation system and used in 5-fold dilutions in gel retardation assay with DNA probes T24, H2B, 68K and IE110 (used at 0.1 nM). Solid single-headed arrows mark the position of free unbound probe while double-headed arrows show the position of protein-DNA complexes.

FIG. 14. Repression of VP16-activated transcription by 6F6-KOX in CAT reporter system. COS-1 cells grown in 6-well cluster dishes are transiently transfected with combinations of pPO13, pCMV-VP16 and pc6F6-KOX (in amounts indicated) and assayed by CAT ELISA (Roche) at 40 h post transfection. ELISA readings (at 405-490 nm) are shown at left hand panel and 6F6-KOX inhibition (right hand panel) is expressed as a percentage of amount of CAT produced in the absence of 6F6-KOX (sample 2). Basal level of CAT produced by pPO13 in the absence of VP16 (sample 1) corresponds to 1%

FIG. 15. Western blot analysis of HSV-1 proteins produced during the course of infection in cells expressing 6F6-KOX and control protein. COS-1 cells, grown in 6-well plate cluster dishes, are transfected either with pc6F6-KOX or pcHIV3-KOX and infected with HIV-1. Additionally transfected but not infected cells, are included into the assay and harvested at the start (mock) and end (m/end) of the experiment. Cell lysates are collected at various times post infection (as indicated) and subjected to SDS-PAGE. Protein samples are transferred onto nitrocellulose and probed for IE175k protein (A), followed by stripping and re-probing with antibodies against IE110k (B) and VP16 (C)

FIG. 16. Inhibition of HSV-1 production by 6F6-KOX. COS-1 cells are transiently transfected with either pTRACER-CMV/Bsd (GFP) or p6F6-KOX-TRACER (6F6-KOX), FACS sorted at 24 h post transfection and GFP and cells infected 24 h later with 0.1 pfu/cell in 24-well cluster dishes. Culture medium samples containing HSV (total of 300 μl) are harvested at 12 h, 22 h and 33.5 h post infection and used for plaque assays on confluent mono-layer of COS cells in 10-fold serial dilutions. After 4 days the cells are fixed in 5% formaldehyde/PBS and stained with 0.1% Toluidine Blue/PBS and number of plaques is counted. The chart shows a total number of infectious particles produced at different time points.

FIG. 17. Detection of HIV-BA′-KOX/c-Myc fusion protein and GFP expression by fluorescent microscopy on transiently transfected or transduced Hela cells. A) Hela cells are used as control. B) Cells are transiently transfected with a pcDNA3.1 expression vector encoding for HIV-BA′-KOX/c-Myc fusion protein. C) Hela cells are transduced with an LNL-based oncoviral vector encoding only for GFP. D) Hela cells are transduced with an LNL-based oncoviral vector encoding for both the HIV-BA′-KOX/c-Myc fusion protein and GFP.

### DETAILED DESCRIPTION OF THE INVENTION

By a combination of rational design and selection, we have produced nucleic acid binding polypeptides in the form of zinc finger proteins which are capable of binding to viral nucleotide sequences. Thus, the nucleic acid binding polypeptides as provided by the present invention are capable of binding to a nucleic acid comprising any viral nucleotide sequence. We further disclose methods which are generally applicable to produce nucleic acid binding polypeptides which are capable of targeting any viral nucleotide sequence, i.e., nucleotide sequences from a wide variety of viruses. Methods of using the nucleic acid binding polypeptides, for example, in therapy, are also disclosed.

As the term is used in this document, a “viral nucleotide sequence” is a nucleotide sequence which comprises, corresponds to, is present in, or is otherwise derived from, any nucleotide sequence which may be found in the genome of a virus. The viral nucleotide sequence may comprise, preferably consist of, 3, 4, 5, 6, 7, 8, 9, 10 or more (preferably contiguous) residues of a nucleotide sequence of a viral genome. Most preferably, the viral nucleotide sequence comprises a nucleotide sequence of 6 or 7 contiguous residues of a nucleotide sequence of a viral genome. A viral promoter sequence further comprises homologues, mutants or derivatives of any of the above sequences, as well as reverse, reverse transcribed or complementary sequences where appropriate (for example, in the case of RNA viruses).

Any viral nucleotide sequence may be targeted. Of particular interest are viral nucleotide sequences which are involved in the regulation of any biological process associated with, linked to, or capable of regulating or controlling, a viral process or function. Preferably, binding of the nucleic acid binding polypeptide to the viral nucleotide sequence modulates the viral process or function. More preferably, such binding modulates the viral process or function in a negative manner, i.e., it reduces, relieves, or represses the function or process. Examples of viral processes and functions include viral titre, binding, infectivity, infection, replication, integration, packaging, transcription, processing, budding, cellular escape, toxicity, growth, etc.

However, the nucleic acid binding polypeptide may, instead of, or in addition, be capable of binding to any nucleotide sequence (such as a nucleotide sequence of a host cell) which is associated with, linked to, or capable of regulating or controlling, any of the above biological processes associated with a viral process or function, so long as such binding is capable of modulating (whether negatively or otherwise) a viral function.

Nucleotide sequences which are involved in the regulation of biological processes and viral processes include sequences involved in viral DNA replication, for example, initiator sequences, origin of replication sequences, promotion of replication sequences (e.g., SV 40 T-antigen sequences), sequences involved in regulation of reverse-transcription, sequences involved in regulation of transcription, sequences involved in regulation of RNA processing, sequences involved in regulation of RNA turnover, sequences involved in regulation of translation, accumulation, transport, intracellular localisation or polypeptide and/or RNA within a cell, sequences involved in regulation of post-transcriptional modification, sequences involved in regulation of activation of a pro-enzyme required for any viral function, sequences involved in regulation of activity of a viral protein, or regulation of breakdown of such a protein, etc. Examples of such sequences are known in the art, and the disclosure of the present invention enables the production of nucleic acid binding polypeptides, capable of binding and regulating such sequences.

Particular target viral nucleotide sequences of interest include viral promoter sequences as well as control sequences and other viral sequences which regulate expression of viral genes and polypeptides. Thus, we disclose nucleic acid binding polypeptides capable of binding nucleic acid sequences comprising a viral promoter sequence, in particular nucleic acid binding polypeptides which are capable of binding to the viral promoter sequence itself. A “viral promoter sequence” may comprise, correspond to, be present in, or be otherwise derived from, a nucleotide sequence present in the promoter of a viral gene. The viral promoter sequence may comprise, preferably consist of, 3, 4, 5, 6, 7, 8, 9, 10 or more (preferably contiguous) residues of a promoter of a viral gene. Most preferably, the viral promoter sequence comprises a nucleotide sequence of 6 or 7 contiguous residues of a promoter of a viral gene. A viral promoter sequence may itself possess viral promoter function or activity, or it may be comprise a sub-sequence of such a sequence. A viral promoter sequence further comprises homologues, mutants or derivatives of any of the above sequences, as well as reverse, reverse transcribed or complementary sequences where appropriate.

We show that such nucleic acid binding polypeptides, optionally coupled with repressor domains (described below) are capable of modulating (in particular, repressing) transcription of a gene linked operatively to the promoter. Preferably, therefore, the nucleic acid binding polypeptides as disclosed here are capable of binding a nucleic acid sequence comprising a viral promoter sequence in such a way as to modulate expression of a gene or reporter operatively linked to the viral promoter sequence. Such polypeptides are therefore useful for regulating transcription of viral and other genes from such promoters. Viral promoters include herpesvirus (e.g., a herpesvirus promoter such as an HSV promoter such as an HSV-1 promoter) and Human Immunodeficiency Virus (e.g., an HIV promoter such as a HIV-1 promoter). Further examples of viruses and their promoters are disclosed below.

Preferably, the polypeptide is capable of binding a promoter of a Immediate Early (IE) gene of HSV-1. Most preferably, the promoter comprises a sequence TAATGARAT, preferably TAATGAGAT. In a highly preferred embodiment, the polypeptides of the invention are capable of repressing transcription from a viral promoter. By the term “repressing”, we mean that the amount of gene transcription from the promoter is reduced, preferably by 10%, 20%, 30%, 40%, 50%, 60%, 70%, 80%, 90%, or 95% or more. Assays for transcriptional and/or promoter activity are well known in the art, and are furthermore described in the Examples. In particular, we describe nucleic acid binding polypeptides which are effective in reducing viral infection. We provide nucleic acid binding polypeptides capable of reducing infection with HIV virus (Examples 8 and 14) as well as those capable of reducing infection with herpesvirus (Example 19). Thus, the nucleic acid binding polypeptides as described here may be used to treat or prevent a disease, condition, or syndrome caused by or associated with viral infection. This is achieved by contacting a cell which is infected by a virus, or which is capable of being infected with a virus, with a pharmaceutically effective amount of nucleic acid binding polypeptide, as disclosed here. The nucleic acid binding polypeptides may also be used to prevent or treat or relieve any of the symptoms associated with these diseases, conditions, etc.

A further application of the zinc fingers disclosed here is in the field of gene therapy for prevention-or treatment of diseases, conditions, syndromes, or the prevention or relief of any of their symptoms. Any of the zinc fingers disclosed here may therefore be introduced into suitable target for such gene therapy, as disclosed in further detail below.

Preferably, the polypeptides according to our invention are isolated or purified. Thus, if the polypeptide is a naturally occurring molecule, then the invention relates to such a molecule only when isolated or purified. The phrase “isolated” or “purified” as used herein means that the molecule is in a context other than its natural context, such as substantially free of one or more components with which it would naturally occur.

Preferably, the polypeptide of the invention is a polypeptide comprising a zinc finger nucleic acid binding motif. Thus, the invention relates in general to a polypeptide molecule wherein the amino acid sequence of said polypeptide comprises a zinc finger motif. The properties of such motifs include the possession of a Cys2-His2 motif, and are discussed in more detail below.

A number of possibilities for the identities of each amino acid at the various positions within the polypeptide are provided. Preferably, more than one amino acid at a given position is selected from amino acids at the positions specified in the tables. Preferably, two, three, four five, six, seven, eight or even more, such as nine amino acids at given positions are selected from amino acids at the positions specified in the above tables. However, ten, twelve, fifteen, eighteen amino acids or even more, such as twenty or twenty one amino acids at given positions may be selected from amino acids at the positions specified in the tables.

The polypeptides according to the invention may be selected for their ability to bind viral promoters, for example, a HIV promoter or a herpesvirus promoter, using the methods described below. A preferred method of selecting such molecules is by phage display. Preferably, the polypeptide molecules are selected by phage display from a library of said phage. This is described in more detail below. We therefore provide a nucleic acid binding molecule capable of binding an HIV (such as an HIV-1) promoter or a herpesvirus (such as an HSV) promoter, said molecule being selected and/or isolated by phage display. As described below, rational design may be used instead of, or in addition to, selection to optimise binding specificity, or affinity, or both, of the nucleic acid binding polypeptide.

We also provide nucleic acid binding polypeptides capable of treating viral infection, optionally in the form of pharmaceutical compositions. Furthermore, they are capable of reducing, preventing, or alleviating the spread of infection of a number of viruses, and may hence be used for treating or preventing diseases associated with or caused by such viruses.

The pharmaceutical compositions provided above may be used for the treatment or therapy of viral infection(s), for example, HIV or related infection(s) or herpesvirus (e.g., HSV) or related infection(s).The term “system” as used here refers to any biological or biochemical system, whether or not whole cells are present. Preferably said system comprised at least part of an organism. In another aspect, the invention relates to a nucleic acid molecule encoding a polypeptide nucleic acid binding molecule as described herein. The nucleic acid may be RNA or DNA.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

Nucleic Acid Binding Polypeptides

This invention relates to nucleic acid binding polypeptides. The term “polypeptide” (and the terms “peptide” and “protein”) are used interchangeably to refer to a polymer of amino acid residues, preferably including naturally occurring amino acid residues. Artificial analogues of amino acids may also be used in the nucleic acid binding polypeptides, to impart the proteins with desired properties or for other reasons. The term “amino acid”, particularly in the context where “any amino acid” is referred to, means any sort of natural or artificial amino acid or amino acid analogue that may be employed in protein construction according to methods known in the art. Moreover, any specific amino acid referred to herein may be replaced by a functional analogue thereof, particularly an artificial functional analogue. Polypeptides may be modified, for example by the addition of carbohydrate residues to form glycoproteins.

As used herein, “nucleic acid” includes both RNA and DNA, constructed from natural nucleic acid bases or synthetic bases, or mixtures thereof. Preferably, however, the binding polypeptides of the invention are DNA binding polypeptides.

Zinc Fingers

Particularly preferred examples of nucleic acid binding polypeptides are Cys2-His2 zinc finger binding proteins which, as is well known in the art, bind to target nucleic acid sequences via α-helical zinc metal atom co-ordinated binding motifs known as zinc fingers. Each zinc finger in a zinc finger nucleic acid binding protein is responsible for determining binding to a nucleic acid triplet, or an overlapping quadruplet, in a nucleic acid binding sequence. Preferably, there are 2 or more zinc fingers, for example 2, 3, 4, 5, 6, 7, 8, 9, 10, 11, 12, 13, 14, 15, 16, 17, 18 or more zinc fingers, in each binding protein. Advantageously, the number of zinc fingers in each zinc finger binding protein is a multiple of 2.

All of the DNA binding residue positions of zinc fingers, as referred to herein, are numbered from the first residue in the α-helix of the finger, ranging from +1 to +9. “−1” refers to the residue in the framework structure immediately preceding the α-helix in a Cys2-His2 zinc finger polypeptide. Residues referred to as “++” are residues present in an adjacent (C-terminal) finger. Where there is no C-terminal adjacent finger, “++” interactions do not operate.

The present invention is in one aspect concerned with the production of what are essentially artificial DNA binding proteins. In these proteins, artificial analogues of amino acids may be used, to impart the proteins with desired properties or for other reasons. Thus, the term “amino acid”, particularly in the context where “any amino acid” is referred to, means any sort of natural or artificial amino acid or amino acid analogue that may be employed in protein construction according to methods known in the art. Moreover, any specific amino acid referred to herein may be replaced by a functional analogue thereof, particularly an artificial functional analogue. The nomenclature used herein therefore specifically comprises within its scope functional analogues or mimetics of the defined amino acids.

The α-helix of a zinc finger binding protein aligns antiparallel to the nucleic acid strand, such that the primary nucleic acid sequence is arranged 3′ to 5′ in order to correspond with the N terminal to C-terminal sequence of the zinc finger. Since nucleic acid sequences are conventionally written 5′ to 3′, and amino acid sequences N-terminus to C-terminus, the result is that when a nucleic acid sequence and a zinc finger protein are aligned according to convention, the primary interaction of the zinc finger is with the −strand of the nucleic acid, since it is this strand which is aligned 3′ to 5′. These conventions are followed in the nomenclature used herein. It should be noted, however, that in nature certain fingers, such as finger 4 of the protein GLI, bind to the +strand of nucleic acid: see Suzuki et al., (1994) NAR 22:3397-3405 and Pavletich and Pabo, (1993) Science 261:1701-1707. The incorporation of such fingers into DNA binding molecules according to the invention is envisaged.

Engineering, Rational and Rule Based Design of Zinc Fingers

The present invention may be integrated with the rules set forth for zinc finger polypeptide design in our European or PCT patent applications having publication numbers; WO 98/53057, WO 98/53060, WO 98/53058, WO 98/53059, describe improved techniques for designing zinc finger polypeptides capable of binding desired nucleic acid sequences. In combination with selection procedures, such as phage display, set forth for example in WO 96/06166, these techniques enable the production of zinc finger polypeptides capable of recognising practically any desired sequence.

We therefore describe a method for preparing a nucleic acid binding protein of the Cys2-His2 zinc finger class capable of binding to a nucleic acid quadruplet in a target nucleic acid sequence comprising a viral nucleotide sequence, wherein binding to each base of the quadruplet by an α-helical zinc finger nucleic acid binding motif in the protein is determined as follows:

(a) if base 4 in the quadruplet is G, then position +6 in the α-helix is Arg or Lys;

(b) if base 4 in the quadruplet is A, then position +6 in the α-helix is Glu, Asn or Val;

(c) if base 4 in the quadruplet is T, then position +6 in the α-helix is Ser, Thr, Val or Lys;

(d) if base 4 in the quadruplet is C, then position +6 in the α-helix is Ser, Thr, Val, Ala, Glu or Asn;

(e) if base 3 in the quadruplet is G, then position +3 in the α-helix is His;

(f) if base 3 in the quadruplet is A, then position +3 in the α-helix is Asn;

(g) if base 3 in the quadruplet is T, then position +3 in the α-helix is Ala, Ser or Val; provided that if it is Ala, then one of the residues at —I or +6 is a small residue;

(h) if base 3 in the quadruplet is C, then position +3 in the α-helix is Ser, Asp, Glu, Leu, Thr or Val;

(i) if base 2 in the quadruplet is G, then position −1 in the α-helix is Arg;

(j) if base 2 in the quadruplet is A, then position −1 in the α-helix is Gln;

(k) if base 2 in the quadruplet is T, then position −1 in the α-helix is His or Thr;

(l) if base 2 in the quadruplet is C, then position −1 in the α-helix is Asp or His.

(m) if base 1 in the quadruplet is G, then position +2 is Glu;

(n) if base 1 in the quadruplet is A, then position +2 Arg or Gln;

(o) if base 1 in the quadruplet is C, then position +2 is Asn, Gln, Arg, His or Lys;

(p) if base 1 in the quadruplet is T, then position +2 is Ser or Thr.

We further describe a method for preparing a nucleic acid binding protein of the Cys2-His2 zinc finger class capable of binding to a nucleic acid quadruplet in a target nucleic acid sequence comprising a viral nucleotide sequence, wherein binding to each base of the quadruplet by an α-helical zinc finger nucleic acid binding motif in the protein is determined as follows:

(a) if base 4 in the quadruplet is G, then position +6 in the α-helix is Arg; or position +6 is Ser or Thr and position ++2 is Asp;

(b) if base 4 in the quadruplet is A, then position +6 in the α-helix is Gln and ++2 is not Asp;

(c) if base 4 in the quadruplet is T, then position +6 in the α-helix is Ser or Thr and position ++2 is Asp;

(d) if base 4 in the quadruplet is C, then position +6 in the α-helix may be any amino acid, provided that position ++2 in the α-helix is not Asp;

(e) if base 3 in the quadruplet is G, then position +3 in the α-helix is His;

(f) if base 3 in the quadruplet is A, then position +3 in the α-helix is Asn;

(g) if base 3 in the quadruplet is T, then position +3 in the α-helix is Ala, Ser or Val; provided that if it is Ala, then one of the residues at —I or +6 is a small residue;

(h) if base 3 in the quadruplet is C, then position +3 in the α-helix is Ser, Asp, Glu, Leu, Thr or Val;

(i) if base 2 in the quadruplet is G, then position −1 in the α-helix is Arg;

(j) if base 2 in the quadruplet is A, then position −1 in the α-helix is Gln;

(k) if base 2 in the quadruplet is T, then position −1 in the α-helix is Asn or Gin;

(l) if base 2 in the quadruplet is C, then position −1 in the α-helix is Asp;

(m) if base 1 in the quadruplet is G, then position +2 is Asp;

(n) if base 1 in the quadruplet is A, then position +2 is not Asp;

(o) if base 1 in the quadruplet is C, then position +2 is not Asp;

(p) if base 1 in the quadruplet is T, then position +2 is Ser or Thr.

The foregoing represents sets of rules which permits the design of a zinc finger binding protein specific for any given target DNA sequence, in particular a viral nucleotide sequence. A zinc finger binding motif is a structure well known to those in the art and defined in, for example, Miller et al., (1985) EMBO J. 4:1609-1614; Berg (1988) PNAS (USA) 85:99-102; Lee et al., (1989) Science 245:635-637; see International patent applications WO 96/06166 and WO 96/32475, corresponding to U.S. Ser. No. 08/422,107, incorporated herein by reference.

In general, a preferred zinc finger framework has the structure:

X₀₋₂ C X₁₋₅ C X₉₋₁₄ H X₃₋₆ H/C

where X is any amino acid, and the numbers in subscript indicate the possible numbers of residues represented by X (Formula A).

The above framework may be further refined to include the structure:

| (A′) X₀₋₂ C X₁₋₅ C X₂₋₇ | X X X X X X X H X₃₋₆ ^{H}/_{C} |
|---|---|
| | −1 1 2 3 4 5 6 7 |

where X is any amino acid, and the numbers in subscript indicate the possible numbers of residues represented by X (Formula A′).

In a preferred aspect of the present invention, zinc finger nucleic acid binding motifs may be represented as motifs having the following primary structure:

| (B) X^{a} C X₂₋₄ C | X X X X L X X H X X X^{b} H - linker |
|---|---|
| X₂₋₃ F X^{c} | −1 1 2 3 4 5 6 7 8 9 |

wherein X (including X^{a}, X^{b} and X^{c}) is any amino acid. X₂₋₄ and X₂₋₃ refer to the presence of 2 or 4, or 2 or 3, amino acids, respectively (Formula B).

The Cys and His residues, which together co-ordinate the zinc metal atom, are marked in bold text and are usually invariant, as is the Leu residue at position +4 in the α-helix.

The linker may comprise a canonical, structured or flexible linker. Structured and flexible linkers (as well as canonical linkers) are described elsewhere in this document, and in our UK application numbers GB 0001582.6, GB0013103.7, GB0013104.5 and our International Patent Application PCT/GB00/00202, all of which are hereby incorporated by reference.

Modifications to this representation may occur or be effected without necessarily abolishing zinc finger function, by insertion, mutation or deletion of amino acids. For example it is known that the second His residue may be replaced by Cys (Krizek et al., (1991) J. Am. Chem. Soc. 113:4518-4523) and that Leu at +4 can in some circumstances be replaced with Arg. The Phe residue before X_{c} may be replaced by any aromatic other than Trp. Moreover, experiments have shown that departure from the preferred structure and residue assignments for the zinc finger are tolerated and may even prove beneficial in binding to certain nucleic acid sequences. Even taking this into account, however, the general structure involving an α-helix co-ordinated by a zinc atom which contacts four Cys or His residues, does not alter. As used herein, structures (A), (A′) and (B) above are taken as an exemplary structure representing all zinc finger-structures of the Cys2-His2 type.

Preferably, X^{a} is F/Y-X or P-F/Y-X. In this context, X is any amino acid. Preferably, in this context X is E, K, T or S. Less preferred but also envisaged are Q, V, A and P. The remaining amino acids remain possible.

Preferably, X₂₋₄ consists of two amino acids rather than four. The first of these amino acids may be any amino acid, but S, E, K, T, P and R are preferred. Advantageously, it is P or R. The second of these amino acids is preferably E, although any amino acid may be used.

Preferably, X^{b} is T or I. Preferably, X^{c} is S or T.

Preferably, X₂₋₃ is G-K-A, G-K-C, G-K-S or G-K-G. However, departures from the preferred residues are possible, for example in the form of M-R-N or M-R.

As set out above, the major binding interactions occur with amino acids −1, +3 and +6. Amino acids +4 and +7 are largely invariant. The remaining amino acids may be essentially any amino acids. Preferably, position +9 is occupied by Arg or Lys. Advantageously, positions +1, +5 and +8 are not hydrophobic amino acids, that is to say are not Phe, Trp or Tyr. Preferably, position ++2 is any amino acid, and preferably serine, save where its nature is dictated by its role as a ++2 amino acid for an N-terminal zinc finger in the same nucleic acid binding molecule.

The code provided by the present invention is not entirely rigid; certain choices are provided. For example, positions +1, +5 and +8 may have any amino acid allocation, whilst other positions may have certain options: for example, the present rules provide that, for binding to a central T residue, any one of Ala, Ser or Val may be used at +3. In its broadest sense, therefore, the present invention provides a very large number of proteins which are capable of binding to every defined target DNA triplet.

Preferably, however, the number of possibilities may be significantly reduced. For example, the non-critical residues +1, +5 and +8 may be occupied by the residues Lys, Thr and Gln respectively as a default option. In the case of the other choices, for example, the first-given option may be employed as a default. Thus, the code according to the present invention allows the design of a single, defined polypeptide (a “default” polypeptide) which will bind to its target triplet. Zinc fingers may be based on naturally occurring zinc fingers and consensus zinc fingers.

In general, naturally occurring zinc fingers may be selected from those fingers for which the DNA binding specificity is known. For example, these may be the fingers for which a crystal structure has been resolved: namely Zif 268 (Elrod-Erickson et al., (1996) Structure 4:1171-1180), GLI (Pavletich and Pabo, (1993) Science 261:1701-1707), Tramtrack (Fairall et al., (1993) Nature 366:483487) and YY1 (Houbaviy et al., (1996) PNAS (USA) 93:13577-13582). Preferably, the modified nucleic acid binding polypeptide is derived from Zif 268, GAC, or a Zif-GAC fusion comprising three fingers from Zif linked to three fingers from GAC. By “GAC-clone”, we mean a three-finger variant of ZIF268 which is capable of binding the sequence GCGGACGCG, as described in Choo & Klug (1994), Proc. Natl. Acad. Sci. USA, 91, 11163-11167.

The naturally occurring zinc finger 2 in Zif 268 makes an excellent starting point from which to engineer a zinc finger and is preferred.

Consensus zinc finger structures may be prepared by comparing the sequences of known zinc fingers, irrespective of whether their binding domain is known. Preferably, the consensus structure is selected from the group consisting of the consensus structure P Y K C P E C G K S F S Q K S D L V K H Q R T H T, and the consensus structure P Y K C S E C G K A F S Q K S N L T R H Q R I H T.

The consensuses are derived from the consensus provided by Krizek et al., (1991) J. Am. Chem. Soc. 113: 45184523 and from Jacobs, (1993) PhD thesis, University of Cambridge, UK. In both cases, canonical, structured or flexible linker sequences, as described below, may be formed on the ends of the consensus for joining two zinc finger domains together.

When the nucleic acid specificity of the model finger selected is known, the mutation of the finger in order to modify its specificity to bind to the target DNA may be directed to residues known to affect binding to bases at which the natural and desired targets differ. Otherwise, mutation of the model fingers should be concentrated upon residues −1, +3, +6 and ++2 as provided for in the foregoing rules.

In order to produce a binding protein having improved binding, moreover, the rules provided by the present invention may be supplemented by physical or virtual modelling of the protein/DNA interface in order to assist in residue selection.

The above rules allow the engineering of a zinc finger capable of binding to a given nucleotide sequence. Engineering of zinc fingers which involves applying rules which specify the choice of amino acid residues based on the identity of residues in a target nucleic acid sequence is referred to here as “rule based” or “rational” design. Such rational design provides a great deal of versatility in zinc finger design.

Selection of Zinc Fingers from Libraries

The rational design described above may be used instead of, or to complement zinc finger production by selection from libraries.

We further describe a method for producing a zinc finger polypeptide capable of binding to a target DNA sequence comprising a viral nucleotide sequence, the method comprising: a) providing a nucleic acid library encoding a repertoire of zinc finger domains or modules, the nucleic acid members of the library being at least partially randomised at one or more of the positions encoding residues −1, 2, 3 and 6 of the α-helix of the zinc finger modules; b) displaying the library in a selection system and screening it against the target DNA sequence; and c) isolating the nucleic acid members of the library encoding zinc finger modules or domains capable of binding to the target sequence.

The term “library” is used according to its common usage in the art, to denote a collection of polypeptides or, preferably, nucleic acids encoding polypeptides. Methods for the production of libraries encoding randomised members such as polypeptides are known in the art and may be applied in the present invention. The members of the library may contain regions of randomisation, such that each library will comprise or encode a repertoire of polypeptides, wherein individual polypeptides differ in sequence from each other. The same principle is present in virtually all libraries developed for selection, such as by phage display.

Randomisation, as used herein, refers to the variation of the sequence of the polypeptides which comprise the library, such that various amino acids may be present at any given position in different polypeptides. Randomisation may be complete, such that any amino acid may be present at a given position, or partial, such that only certain amino acids are present. Preferably, the randomisation is achieved by mutagenesis at the nucleic acid level, for example by synthesising novel genes encoding mutant proteins and expressing these to obtain a variety of different proteins. Alternatively, existing genes can be themselves mutated, such by site-directed or random mutagenesis, in order to obtain the desired mutant genes.

Zinc finger polypeptides may be designed which specifically bind to nucleic acids incorporating the base U, in preference to the equivalent base T.

In a further preferred aspect, the invention comprises a method for producing a zinc finger polypeptide capable of binding to a target DNA sequence comprising a viral nucleotide sequence, the method comprising: a) providing a nucleic acid library encoding a repertoire of zinc finger polypeptides each possessing more than one zinc finger, the nucleic acid members of the library being at least partially randomised at one or more of the positions encoding residues −1, 2, 3 and 6 of the α-helix in a first zinc finger and at one or more of the positions encoding residues −1, 2, 3 and 6 of the α-helix in a further zinc finger of the zinc finger polypeptides; b) displaying the library in a selection system and screening it against the target DNA sequence; and d) isolating the nucleic acid members of the library encoding zinc finger polypeptides capable of binding to the target sequence.

In this aspect, the invention encompasses library technology described in our International patent application WO 98/53057, incorporated herein by reference in its entirety. WO 98/53057 describes the production of zinc finger polypeptide libraries in which each individual zinc finger polypeptide comprises more than one, for example two or three, zinc fingers; and wherein within each polypeptide partial randomisation occurs in at least two zinc fingers. This allows for the selection of the “overlap” specificity, wherein, within each triplet, the choice of residue for binding to the third nucleotide (read 3′ to 5′ on the +strand) is influenced by the residue present at position +2 on the subsequent zinc finger, which displays cross-strand specificity in binding. The selection of zinc finger polypeptides incorporating cross-strand specificity of adjacent zinc fingers enables the selection of nucleic acid binding proteins more quickly, and/or with a higher degree of specificity than is otherwise possible.

Zinc finger binding motifs designed according to the invention may be combined into nucleic acid binding polypeptide molecules having a multiplicity of zinc fingers. Preferably, the proteins have at least two zinc fingers. The presence of at least three zinc fingers is preferred. Nucleic acid binding proteins may be constructed by joining the required fingers end to end, N-terminus to C-terminus, with canonical, flexible or structured linkers, as described below. Preferably, this is effected by joining together the relevant nucleic acid sequences which encode the zinc fingers to produce a composite nucleic acid coding sequence encoding the entire binding protein.

The invention therefore provides a method for producing a DNA binding protein as defined above, wherein the DNA binding protein is constructed by recombinant DNA technology, the method comprising the steps of: preparing a nucleic acid coding sequence encoding a plurality of zinc finger domains or modules defined above, inserting the nucleic acid sequence into a suitable expression vector; and expressing the nucleic acid sequence in a host organism in order to obtain the DNA binding protein. A “leader” peptide may be added to the N-terminal finger. Preferably, the leader peptide is MAEEKP.

Multifinger Polypeptides

According to a preferred embodiment of the present invention, the nucleic acid binding polypeptides comprise a plurality of binding domains or motifs. For example, a preferred zinc finger polypeptide according to the invention comprises 2, 3, 4, 5, 6, 7, 8, 9, 10, 11, 12, 13, 14, 15, 16, 17, 18, 19, 20, 21, 22, 23, 24, etc or more zinc finger binding domains or motifs. Highly preferred embodiments are zinc finger polypeptides which comprise three zinc finger motifs and those which comprise six finger motifs.

Zinc finger polypeptides comprising multiple fingers may be constructed by joining together two or more zinc finger polypeptides (which may themselves be selected using phage display, as described elsewhere in this document) with suitable linker sequences. Preferred linker sequences comprise flexible linkers, structured linkers, combined linkers or any combination of these, as described in further detail below.

Means of joining polypeptide sequences, for example, by recombinant DNA technology are known in the art, and are for example disclosed in Sambrook et al (supra) and Ausubel et al (supra). Furthermore, other sequences such as nuclear localisation sequences and “tag” sequences for purification may be included as known in the art. A specific example of production of a six finger protein 6F6 is described in the Examples below, which also describe production of six finger proteins comprising repressor domains (for example, 6F6-KOX).

Flexible and Structured Linkers

The nucleic acid binding polypeptides according to the invention may comprise one or more linker sequences. The linker sequences may comprise one or more flexible linkers, one or more structured linkers, or any combination of flexible and structured linkers. Such linkers are disclosed in our co-pending British Patent Application Numbers 0001582.6, 0013102.9, 0013103.7, 0013104.5 and International Patent Application Number PCT/GB01/00202, which are incorporated by reference.

By “linker sequence” we mean an amino acid sequence that links together two nucleic acid binding modules. For example, in a “wild type” zinc finger protein, the linker sequence is the amino acid sequence lacking secondary structure which lies between the last residue of the α-helix in a zinc finger and the first residue of the β-sheet in the next zinc finger. The linker sequence therefore joins together two zinc fingers. Typically, the last amino acid in a zinc finger is a threonine residue, which caps the α-helix of the zinc finger, while a tyrosine/phenylalanine or another hydrophobic residue is the first amino acid of the following zinc finger. Accordingly, in a “wild type” zinc finger, glycine is the first residue in the linker, and proline is the last residue of the linker. Thus, for example, in the Zif268 construct, the linker sequence is G(E/Q)(K/R)P.

A “flexible” linker is an amino acid sequence which does not have a fixed structure (secondary or tertiary structure) in solution. Such a flexible linker is therefore free to adopt a variety of conformations. An example of a flexible linker is the canonical linker sequence GERP/GEKP/GQRP/GQKP. Flexible linkers are also disclosed in WO99/45132 (Kim and Pabo). By “structured linker” we mean an amino acid sequence which adopts a relatively well-defined conformation when in solution Structured linkers are therefore those which have a particular secondary and/or tertiary structure in solution.

Determination of whether a particular sequence adopts a structure may be done in various ways, for example, by sequence analysis to identify residues likely to participate in protein folding, by comparison to amino acid sequences which are known to adopt certain conformations (e.g., known alphα-helix, beta-sheet or zinc finger sequences), by NMR spectroscopy, by X-ray diffraction of crystallised peptide containing the sequence, etc as known in the art.

The structured linkers of our invention preferably do not bind nucleic acid, but where they do, then such binding is not sequence specific. Binding specificity may be assayed for example by gel-shift as described below.

The linker may comprise any amino acid sequence that does not substantially hinder interaction of the nucleic acid binding modules with their respective target subsites. Preferred amino acid residues for flexible linker sequences include, but are not limited to, glycine, alanine, serine, threonine proline, lysine, arginine, glutamine and glutamic acid.

The linker sequences between the nucleic acid binding domains preferably comprise five or more amino acid residues. The flexible linker sequences according to our invention consist of 5 or more residues, preferably, 5, 6, 7, 8, 9, 10, 11, 12, 13, 14, 15, 16, 17, 18, 19 or 20 or more residues. In a highly preferred embodiment of the invention, the flexible linker sequences consist of 5, 7 or 10 residues.

Once the length of the amino acid sequence has been selected, the sequence of the linker may be selected, for example by phage display technology (see for example U.S. Pat. No. 5,260,203) or using naturally occurring or synthetic linker sequences as a scaffold (for example, GQKP and GEKP, see Liu et al., 1997, Proc. Natl. Acad. Sci. USA 94, 5525-5530 and Whitlow et al., 1991, Methods: A Companion to Methods in Enzymology 2: 97-105). The linker sequence may be provided by insertion of one or more amino acid residues into an existing linker sequence of the nucleic acid binding polypeptide. The inserted residues may include glycine and/or serine residues. Preferably, the existing linker sequence is a canonical linker sequence selected from GEKP, GERP, GQKP and GQRP. More preferably, each of the linker sequences comprises a sequence selected from GGEKP, GGQKP, GGSGEKP, GGSGQKP, GGSGGSGEKP, and GGSGGSGQKP.

Structured linker sequences are typically of a size sufficient to confer secondary or tertiary structure to the linker; such linkers may be up to 30, 40 or 50 amino acids long. In a preferred embodiment, the structured linkers are derived from known zinc fingers which do not bind nucleic acid, or are not capable of binding nucleic acid specifically. An example of a structured linker of the first type is TFIIIA finger IV; the crystal structure of TFIIIA has been solved, and this shows that finger IV does not contact the nucleic acid (Nolte et al., 1998, Proc. Natl. Acad. Sci. USA 95, 2938-2943.). An example of the latter type of structured linker is a zinc finger which has been mutagenised at one or more of its base contacting residues to abolish its specific nucleic acid binding capability. Thus, for example, a ZIF finger 2 which has residues −1, 2, 3 and 6 of the recognition helix mutated to serines so that it no longer specifically binds DNA may be used as a structured linker to link two nucleic acid binding domains.

The use of structured or rigid linkers to jump the minor groove of DNA is likely to be especially beneficial in (i) linking zinc fingers that bind to widely separated (>3 bp) DNA sequences, and (ii) also in minimising the loss of binding energy due to entropic factors.

Typically, the linkers are made using recombinant nucleic acids encoding the linker and the nucleic acid binding modules, which are fused via the linker amino acid sequence. The linkers may also be made using peptide synthesis and then linked to the nucleic acid binding modules. Methods of manipulating nucleic acids and peptide synthesis methods are known in the art (see, for example, Maniatis, et al., 1991. Molecular Cloning: A Laboratory Manual. Cold Spring Harbor, N.Y., Cold Spring Harbor Laboratory Press).

repressors

According to a further aspect of our invention, we provide a nucleic acid binding polypeptide comprising a repressor domain and one or more nucleic acid binding domains. The repressor domain is preferably a transcriptional repressor domain selected from the group consisting of: a KRAB-A domain, an engrailed domain and a snag domain. Such a nucleic acid binding polypeptide may comprise nucleic acid binding domains linked by at least one flexible linker, one or more domains linked by at least one structured linker, or both.

The nucleic acid binding polypeptides according to our invention may be linked to one or more transcriptional effector domains, such as an activation domain or a repressor domain. Examples of transcriptional activation domains include the VP16 and VP64 transactivation domains of Herpes Simplex Virus. Alternative transactivation domains are various and include the maize C1 transactivation domain sequence (Sainz et al., 1997, Mol. Cell. Biol. 17: 115-22) and P1 (Goff et al., 1992, Genes Dev. 6: 864-75; Estruch et al., 1994, Nucleic Acids Res. 22: 3983-89) and a number of other domains that have-been reported from plants (see Estruch et al, 1994, ibid).

Instead of incorporating a transactivator of gene expression, a repressor of gene expression can be fused to the nucleic acid binding polypeptide and used to down regulate the expression of a gene contiguous or incorporating the nucleic acid binding polypeptide target sequence. Such repressors are known in the art and include, for example, the KRAB-A domain (Moosmann et al., Biol. Chem. 378: 669-677 (1997)), the KRAB domain from human KOX1 protein (Margolin et al., PNAS 91:45094513 (1994)), the engrailed domain (Han et al., Embo J. 12: 2723-2733 (1993)) and the snag domain (Grimes et al., Mol Cell. Biol. 16: 6263-6272 (1996)). These can be used alone or in combination to down-regulate gene expression.

Molecules according to the invention comprising zinc finger proteins may be fused to transcriptional repression domains such as the Kruppel-associated box (KRAB) domain to form powerful repressors. These fusions are known to repress expression of a reporter gene even when bound to sites a few kilobase pairs upstream from the promoter of the gene (Margolin et al., 1994, PNAS USA 91, 4509-4513).

Virus

The virus targeted by a nucleic acid binding polypeptide according to the invention may be an RNA virus or a DNA virus. Preferably, the virus is an integrating virus. Preferably, the virus is selected from a lentivirus and a herpesvirus. More preferably, the virus is an HIV virus or a HSV virus. The methods described here can therefore be used to prevent the development and establishment of diseases caused by or associated with any of the above viruses, including human immunodeficiency virus, such as HIV-1 and HIV-2, and herpesvirus, for example HSV-1, HSV-2, HSV-7 and HSV-8, as well as human cytomegalovirus, varicella-zoster virus, Epstein-Barr virus and human herpesvirus 6.in humans.

Examples of viruses which may be targeted using the present invention are given in the tables below.

DNA VIRUSES

| | Genus or | | |
|---|---|---|---|
| Family | [Subfamily] | Example | Diseases |
| Herpesviridae | [Alphaherpes- | Herpes simplex virus type 1 | Encephalitis, cold sores, gingivostomatitis |
| | virinae] | (aka HHV-1) | |
| | | Herpes simplex virus type 2 | Genital herpes, encephalitis |
| | | (aka HHV-2) | |
| | | Varicella zoster virus (aka | Chickenpox, shingles |
| | | HHV-3) | |
| | [Gammaherpesvirinae] | Epstein Barr virus (aka HHV- | Mononucleoisis, hepatitis, tumors (BL, NPC) |
| | | 4) | |
| | | Kaposi's sarcoma associated | ?Probably: tumors, inc. Kaposi's sarcoma |
| | | herpesvirus, KSHV (aka | (KS) and some B cell lymphomas |
| | | Human herpesvirus 8) | |
| | [Betaherpesvirinae] | Human cytomegalovirus (aka | Mononucleosis, hepatitis, pneumonitis, |
| | | HHV-5) | congenital |
| | | Human herpesvirus 6 | Roseola (aka E. subitum), pneumonitis |
| Adenoviridae | | Human herpesvirus 7 | Some cases of roseola? |
| Papovaviridae | Mastadenovirus | Human adenoviruses | 50 serotypes (species); respiratory infections |
| | Papillomavirus | Human papillomaviruses | 80 species; warts and tumors |
| Hepadnaviridae | Polyomavirus | JC, BK viruses | Mild usually; JC causes PML in AIDS |
| Poxviridae | Orthohepadnavirus | Hepatitis B virus (HBV) | Hepatitis (chronic), cirrhosis, liver tumors |
| | | Hepatitis C virus (HCV) | Hepatitis (chronic), cirrhosis, liver tumors |
| | Orthopoxvirus | Vaccinia virus | Smallpox vaccine virus |
| | | Monkeypox virus | Smallpox-like disease; a rare zoonosis (recent |
| | | | outbreak in Congo; 92 cases from February 1996-February 1997) |
| Parvoviridae | Parapoxvirus | Orf virus | Skin lesions (“pocks”) |
| | Erythrovirus | B19 parvovirus | E. infectiousum (aka Fifth disease), aplastic |
| | | | crisis, fetal loss |
| Circoviridae | Dependovirus | Adeno-associated | Useful for gene therapy; integrates into |
| | Circovirus | TT virus (TTV) | chromosome |
| | | | Linked to hepatitis of unknown etiology |
| Picornaviridae | Enterovirus | Polioviruses | 3 types; Aseptic meningitis, paralytic |
| | | | poliomyelitis |
| | | Echoviruses | 30 types; Aseptic meningitis, rashes |
| | | Coxsackieviruses | 30 types; Aseptic meningitis, myopericarditis |
| | Hepatovirus | Hepatitis A virus | Acute hepatitis (fecal-oral spread) |
| | Rhinovirus | Human rhinoviruses | 115 types; Common cold |
| Caliciviridae | Calicivirus | Norwalk virus | Gastrointestinal illness |
| Paramyxoviridae | Paramyxovirus | Parainfluenza viruses | 4 types; Common cold, bronchiolitis, |
| | | | pneumonia |
| | Rubulavirus | Mumps virus | Mumps: parotitis, aseptic meningitis (rare: |
| | | | orchitis, encephalitis) |
| | Morbillivirus | Measles virus | Measles: fever, rash (rare: encephalitis, |
| | | | SSPE) |
| | Pneumovirus | Respiratory syncytial virus | Common cold (adults), bronchiolitis, |
| | | | pneumonia (infants) |
| Orthomyxo- | Influenzavirus A | Influenza virus A | Flu: fever, myalgia, malaise, cough, |
| viridae | | | pneumonia |
| | Influenzavirus B | Influenza virus B | Flu: fever, myalgia, malaise, cough, |
| | | | pneumonia |
| Rhabdoviridae | Lyssavirus | Rabies virus | Rabies: long incubation, then CNS disease, |
| | | | death |
| Filoviridae | Filovirus | Ebola and Marburg viruses | Hemorrhagic fever, death |
| Bornaviridae | Bornavirus | Borna disease virus | Uncertain; linked to schizophrenia-like |
| | | | disease in some animals |
| Retroviridae | Deltaretrovirus | Human T-lymphotropic virus | Adult T-cell leukemia (ATL), tropical spastic |
| | | type-1 | paraparesis (TSP) |
| | Spumavirus | Human foamy viruses | No disease known |
| | Lentivirus | Human immunodeficiency | AIDS, CNS disease |
| | | virus type-1 and -2 | |
| Togaviridae | Rubivirus | Rubella virus | Mild exanthem; congenital fetal defects |
| | Alphavirus | Equine encephalitis viruses | Mosquito-born, encephalitis |
| | | (WEE, EEE, VEE) | |
| Flaviviridae | Flavivirus | Yellow fever virus | Mosquito-born; fever, hepatitis (yellow |
| | | | fever!) |
| | | Dengue virus | Mosquito-born; hemorrhagic fever |
| | | St. Louis Encephalitis virus | Mosquito-born; encephalitis |
| | Hepacivirus | Hepatitis C virus | Hepatitis (often chronic), liver cancer |
| | | Hepatitis G virus | Hepatitis??? |
| Reoviridae | Rotavirus | Human rotaviruses | Numerous serotypes; Diarrhea |
| | Coltivirus | Colorado Tick Fever virus | Tick-born; fever |
| | Orthoreovirus | Human reoviruses | Minimal disease |
| Bunyaviridae | Hantavirus | Pulmonary Syndrome | Rodent spread; pulmonary illness (can be |
| | | Hantavirus | letbal, “Four Corners” outbreak) |
| | | Hantaan virus | Rodent spread; hemorrhagic fever with renal |
| | | | syndrome |
| | Phlebovirus | Rift Valley Fever virus | Mosquito-born; hemorrhagic fever |
| | Nairovirus | Crimean-Congo Hemorrhagic | Mosquito-born; hemorrhagic fever |
| | | Fever virus | |
| Arenaviridae | Arenavirus | Lymphocytic | Rodent-born; fever, aseptic meningitis |
| | | Choriomeningitis virus | |
| | | Lassa virus | Rodent-born; severe hemorrhagic fever (BL4 |
| | | | agents; also: Machupo, Junin) |
| | Deltavirus | Hepatitis Delta virus | Requires HBV to grow; hepatitis, liver cancer |
| Coronaviridae | Coronavirus | Human coronaviruses | Mild common cold-like illness |
| Astroviridae | Astrovirus | Human astroviruses | Gastroenteritis |
| Unclassified | “Hepatitis E-like | Hepatitis E virus | Hepatitis (acute); fecal-oral spread |
| | viruses” | | |

Human Immunodeficiency Virus-1 (HIV-1)

The nucleic acid binding polypeptides of the present invention are capable of binding to nucleic acid sequences comprising or derived from Human Immunodeficiency Virus (HIV) nucleotide sequences. We also provide nucleic acid binding polypeptides capable of treating HIV infection. The methods described here can therefore be used to prevent the development and establishment of diseases caused by or associated with human immunodeficiency virus, such as HIV-1 and HIV-2.

Human Immunodeficiency Virus (HIV) is a retrovirus which infects cells of the immune system, most importantly CD4⁺ T lymphocytes. CD4⁺ T lymphocytes are important, not only in terms of their direct role in immune function, but also in stimulating normal function in other components of the immune system, including CD8⁺ T-lymphocytes. These HIV infected cells have their function disturbed by several mechanisms and/or are rapidly killed by viral replication. The end result of chronic HIV infection is gradual depletion of CD4⁺ T lymphocytes, reduced immune capacity, and ultimately the development of AIDS, leading to death.

The regulation of HIV gene expression is accomplished by a combination of both cellular and viral factors. HIV gene expression is regulated at both the transcriptional and post-transcriptional levels. The HIV genes can be divided into the early genes and the late genes. The early genes, Tat, Rev, and Nef, are expressed in a Rev-independent manner. The mRNAs encoding the late genes, Gag, Pol, Env, Vpr, Vpu, and Vif require Rev to be cytoplasmically localized and expressed. HIV transcription is mediated by a single promoter in the 5′ LTR. Expression from the 5′ LTR generates a 9-kb primary transcript that has the potential to encode all nine HIV genes. The primary transcript is roughly 600 bases shorter than the provirus. The primary transcript can be spliced into one of more than 30 mRNA species or packaged without further modification into virion particles (to serve as the viral RNA genome).

Transcription of the HIV genome beginning from the HIV-1 promoter is an important event in the lifecycle of HIV. Modulation of this activity is useful both in terms of studying HIV and in development of therapeutics in order to combat it. Nucleic acid binding molecules which bind specifically to this region will therefore be useful in these and other applications. Disclosed herein are nucleic acid binding molecules which specifically target the HIV-1 promoter. Preferably, these molecules comprise polypeptides.

In one particular embodiment of the invention, we disclose a polypeptide capable of binding to a nucleic acid comprising a sequence present in the Human Immunodeficiency Virus-I (HIV-1) promoter, in which the polypeptide comprises three zinc fingers F1, F2 and F3, at least one of the amino acids at positions −1, 3 and 6 of F1, −1, 3 and 6 of F2 and −1, 3 and 6 of F3 being selected from amino acids specified in the following table:

| F1: amino acid | |
|---|---|
| −1 | R, D, A, H |
| 3 | E, H, D, S, A, V |
| 6 | R, K, Q |
| F2 | |
| −1 | R, N, Q, D |
| 3 | N, H, D |
| 6 | T, R, K |
| F3 | |
| −1 | R, D, T, Q, A |
| 3 | H, N, T, S, V |
| 6 | T, K, R |

In a further embodiment, the polypeptide comprises three zinc fingers F1, F2 and F3, and at least one of the amino acids at positions −1, 1, 2, 3, 4, 5 and 6 of F1, −1, 1, 2, 3, 4, 5 and 6 of F2 and −1, 1, 2, 3, 4, 5 and 6 of F3 is selected from amino acids specified in the following table:

| F1: amino acid | |
|---|---|
| −1 | R, D, A, H |
| 1 | S |
| 2 | D, A, S |
| 3 | E, H, D, S, A, V |
| 4 | L |
| 5 | T, I |
| 6 | R, K, Q |
| F2 | |
| −1 | R, N, Q, D |
| 1 | S, R |
| 2 | D, S, A |
| 3 | N, H, D |
| 4 | L |
| 5 | S, T |
| 6 | T, R, K |
| F3 | |
| −1 | R, D, T, Q, A |
| 1 | R, S, N, Y |
| 2 | D, A, S |
| 3 | H, N, T, S, V |
| 4 | R |
| 5 | T, K |
| 6 | T, K, R |

Preferably, each of the amino acids at the numbered positions are selected from amino acids specified in the table.

In a preferred embodiment of the invention, a nucleic acid binding polypeptide capable of binding a human immunodeficiency virus nucleotide sequence comprises one or more of the following sequences:

| SEQ | | |
|---|---|---|
| ID | | |
| NO: | Sequence | Name |
| | X₀₋₂ C X₁₋₅ C X₂₋₇ R S D E L T R H X₃₋₆ ^{H}/_{C} | HIV-A F1 |
| | X₀₋₂ C X₁₋₅ C X₂₋₇ R S D N L S T H X₃₋₆ ^{H}/_{C} | HIV-A F2 |
| | X₀₋₂ C X₁₋₅ C X₂₋₇ R R D H R T T H X₃₋₆ ^{H}/_{C} | HIV-A F3 |
| | X₀₋₂ C X₁₋₅ C X₂₋₇ R S D V L T R H X₃₋₆ ^{H}/_{C} | HIV-A′F1 |
| | X₀₋₂ C X₁₋₅ C X₂₋₇ R S D H L T T H X₃₋₆ ^{H}/_{C} | HIV-A′F2 |
| | X₀₋₂ C X₁₋₅ C X₂₋₇ D Y S V R K R H X₃₋₆ ^{H}/_{C} | HIV-A′F3 |
| | X₀₋₂ C X₁₋₅ C X₂₋₇ D S A H L T R H X₃₋₆ ^{H}/_{C} | HIV-B F1 |
| | X₀₋₂ C X₁₋₅ C X₂₋₇ R S D H L S T H X₃₋₆ ^{H}/_{C} | HIV-B F2 |
| | X₀₋₂ C X₁₋₅ C X₂₋₇ D S A N R T K H X₃₋₆ ^{H}/_{C} | HIV-B F3 |
| | X₀₋₂ C X₁₋₅ C X₂₋₇ A S A D L T R H X₃₋₆ ^{H}/_{C} | HIV-C F1 |
| | X₀₋₂ C X₁₋₅ C X₂₋₇ N R S D L S R H X₃₋₆ ^{H}/_{C} | HIV-C F2 |
| | X₀₋₂ C X₁₋₅ C X₂₋₇ T S S N R K K H X₃₋₆ ^{H}/_{C} | HIV-C F3 |
| | X₀₋₂ C X₁₋₅ C X₂₋₇ H S S D L T R H X₃₋₆ ^{H}/_{C} | HIV-D F1 |
| | X₀₋₂ C X₁₋₅ C X₂₋₇ Q S S D L S K H X₃₋₆ ^{H}/_{C} | HIV-D F2 |
| | X₀₋₂ C X₁₋₅ C X₂₋₇ Q N A T R K R H X₃₋₆ ^{H}/_{C} | HIV-D F3 |
| | X₀₋₂ C X₁₋₅ C X₂₋₇ D S S S L T K H X₃₋₆ ^{H}/_{C} | HIV-E F1 |
| | X₀₋₂ C X₁₋₅ C X₂₋₇ Q S A H L S T H X₃₋₆ ^{H}/_{C} | HIV-E F2 |
| | X₀₋₂ C X₁₋₅ C X₂₋₇ D S S S R T K H X₃₋₆ ^{H}/_{C} | HIV-E F3 |
| | X₀₋₂ C X₁₋₅ C X₂₋₇ A S D D L T Q H X₃₋₆ ^{H}/_{C} | HIV-F F1 |
| | X₀₋₂ C X₁₋₅ C X₂₋₇ R S S D L S R H X₃₋₆ ^{H}/_{C} | HIV-F F2 |
| | X₀₋₂ C X₁₋₅ C X₂₋₇ Q S A H R T K H X₃₋₆ ^{H}/_{C} | HIV-F F3 |
| | X₀₋₂ C X₁₋₅ C X₂₋₇ R S D A L I Q H X₃₋₆ ^{H}/_{C} | HIV-G F1 |
| | X₀₋₂ C X₁₋₅ C X₂₋₇ D R A N L S T H X₃₋₆ ^{H}/_{C} | HIV-G F2 |
| | X₀₋₂ C X₁₋₅ C X₂₋₇ A S S T R T K H X₃₋₆ ^{H}/_{C} | HIV-G F3 |
| | X₀₋₂ C X₁₋₅ C X₂₋₇ R S D E L T R H X₃₋₆ ^{H}/_{C -} | HIV-A |
| | linker - X₀₋₂ C X₁₋₅ C X₂₋₇ R S D N L S T H | |
| | X₃₋₆ ^{H}/_{C} - linker - X₀₋₂ C X₁₋₅ C X₂₋₇ D S A N R | |
| | T K H X₃₋₆ ^{H}/_{C} | |
| | MAERPYACPVESCDRRFSRSDVLTRHIRIHTGQKPFQCRICM | HIV-A′A |
| | RNFSRSDHLTTHIRTHTGEKPFACDICGRKFADYSVRKRHTK | |
| | IHTGGSGGSGERPYACPVESCDRRFSRSDELTRHIRIHTGQK | |
| | PFQCRICMRNFSRSDNLSTHIRTHTGEKPFACDICGRKFARR | |
| | DHRTTHTKIHL | |
| | MAERPYACPVESCDRRFSDSAHLTRHIRIHTGQKPFQCRICM | HIV-BA |
| | RNFSRSDHLSTHIRTHTGEKPFACDICGRKFADSANRTKHTK | |
| | IHLRQKDGGSGGSGGSGGSGGSGGSERPYACPVESCDRRFSR | |
| | SDELTRHIRIHTGQKPFQCRICMRNFSRSDNLSTHIRTHTGE | |
| | KPFACDICGRKFARRDHRTTHTKIH | |
| | MAERPYACPVESCDRRFSDSAHLTRHIRIHTGQKPFQCRICM | HIV-BA′ |
| | RNFSRSDHLSTHIRTHTGEKFPACDICGRKFADSANRTKHTK | |
| | IHTGGSGERPYACPVESCDRRFSRSDVLTRHIRIHTGQKPFQ | |
| | CRICMRNFSRSDHLTTHIRTHTGEKPFACDICGRKFADYSVR | |
| | KRHTKIH | |
| | MAERPYACPVESCDRRFSRSDVLTRHIRIHTGQKPFQCRICM | HIV-A′A-KOX |
| | RNFSRSDHLTTHIRTHTGEKPFACDICGRKFADYSVRKRHTK | |
| | IHTGGSGGSGERPYACPVESCDRRFSRSDELTRHIRIHTGQK | |
| | PFQCRICMRNFSRSDNLSTHIRTHTGEKPFACDICGRKFARR | |
| | DHRTTHTKIHLRQKDAARNSGPKKKRKVDGGGALSPQHSAVT | |
| | QGSIIKNKEGMDAKSLTAWSRTLVTFKDVFVDFTREEWKLLD | |
| | TAQQIVYRNVMLENYKNLVSLGYQLTKPDVILRLEKGEEPWL | |
| | VEREIHQETHPDSETAFEIKSSVEQKLISEEDL | |
| | MAERPYACPVESCDRRFSDSAHLTRHIRIHTGQKPFQCRICM | HIV-BA-KOX |
| | RNFSRSDHLSTHIRTHTGEKPFACDICGRKFADSANRTKHTK | |
| | IHLRQKDGGSGGSGGSGGSGGSGGSERPYACPVESCDRRFSR | |
| | SDELTRHIRIHTGQKPFQCRICMRNFSRSDNLSTHIRTHTGE | |
| | KPFACDICGRKFARRDHRTTHTKIHLRQKDAARNSGPKKKRK | |
| | VDGGGALSPQHSAVTQGSIIKNKEGMDAKSLTAWSRTLVTFK | |
| | DVFVDFTREEWKLLDTAQQIVYRNVMLENYKNLVSLGYQLTK | |
| | PDVILRLEKGEEPWLVEREIHQETHPDSETAFEIKSSVEQKL | |
| | ISEEDL | |
| | MAERPYACPVESCDRRFSDSAHLTRHIRIHTGQKPFQCRICM | HIV-BA′-KOX |
| | RNFSRSDHLSTHIRTHTGEKPFACDICGRKFADSANRTKHTK | |
| | IHTGGSGERPYACPVESCDRRFSRSDVLTRHIRIHTGQKPFQ | |
| | CRICMRNFSRSDHLTTHIRTHTGEKPFACDICGRKFADYSVR | |
| | KRHTKIHLRQKDAARNSGPKKKRKVDGGGALSPQHSAVTQGS | |
| | IIKNKEGMDAKSLTAWSRTLVTFKDVFVDFTREEWKLLDTAQ | |
| | QIVYRNVMLENYKNLVSLGYQLTKPDVILRLEKGEEPWLVER | |
| | EIHQETHPDSETAFEIKSSVEQKLISEEDL | |

Herpes Virus

The nucleic acid binding polypeptides of the present invention are capable of binding to nucleic acid sequences comprising or derived from Herpesvirus nucleotide sequences, we also provide nucleic acid binding polypeptides capable of treating Herpesvirus infection. The methods described here can therefore be used to prevent the development and establishment of diseases caused by or associated with herpesvirus, for example HSV-1, HSV-2, HSV-7 and HSV-8.

Particular examples of herpesvirus include: herpes simplex virus I (“HSV-1”), herpes simplex virus 2 (“HSV-2”), human cytomegalovirus (“HCMV”), varicella-zoster virus (“VZV”), Epstein-Barr virus (“EBV”), human herpesvirus 6 (“HHV6”), herpes simplex virus 7 (“HSV-7”) and herpes simplex virus 8 (“HSV-8”).

Herpesviruses have also been isolated from horses, cattle, pigs (pseudorabies virus (“PSV”) and porcine cytomegalovirus), chickens (infectious larygotracheitis), chimpanzees, birds (Marck's disease herpesvirus 1 and 2), turkeys and fish (see “Herpesviridae: A Brief Introduction”, Virology, Second Edition, edited by B; N. Fields, Chapter 64,1787 (1990)).

Herpes simplex viral (“HSV”) infection is generally a recurrent viral infection characterized by the appearance on the skin or mucous membranes of single or multiple clusters of small vesicles, filled with clear fluid, on slightly raised inflammatory bases. The herpes simplex virus is a relatively large-sized virus. HSV-2 commonly causes herpes labialis. HSV-2 is usually, though not always, recoverable from genital lesions. Ordinarily, HSV-2 is transmitted venereally.

Diseases caused by varicella-zoster virus (human herpesvirus 3) include varicella (chickenpox) and zoster (shingles). Cytomegalovirus (human herpesvirus 5) is responsible for cytomegalic inclusion disease in infants. There is presently no specific treatment for treating patients infected with cytomegalovirus. Epstein-Barr virus (human herpesvirus 4) is the causative agent of infectious mononucleosis and has been associated with Burkitt's lymphoma and nasopharyngeal carcinoma. Animal herpesviruses which may pose a problem for humans include B virus (herpesvirus of Old World Monkeys) and Marmoset herpesvirus (herpesvirus of New World Monkeys).

Herpes simplex virus 1 (HSV-1) is a human pathogen capable of becoming latent in nerve cells. Like all the other members of Herpesviridae it has a complex architecture and double-stranded linear DNA genome which encodes for variety of viral proteins including DNA pol and TK (FIG. 8).

HSV gene expression proceeds in a sequential and strictly regulated manner and can be divided into at least three phases, termed immediate-early (IE or α), early (β) and late (γ) (FIG. 8). The cascade of HSV-1 gene expression starts from IE genes, which are expressed immediately after lytic infection begins. The IE proteins regulate the expression of later classes of genes (early and late) as well as their own expression. The product of IE175k (ICP4) gene is critical for HSV-1 gene regulation and ts mutants in this gene are blocked at IE stage of infection.

The IE genes themselves are activated by a virion structural protein VP 16 (expressed late in the replicative cycle and incorporated into HSV particle). All 5 IE genes of HSV-1 (IE110k-2 copies/HSV genome, IE175-2 copies/HSV genome, IE68k, IE63k and IE12k) have at least one copy of a conserved promoter/enhancer sequence—TAATGARAT. This sequence is recognized by the transactivation complex which consists of; Oct-1, HCF and VP16 (FIG. 9). The GARAT element is required for efficient transactivation by VP16. This mechanism of gene activation is unique for HSV and despite Oct-1 being a common transcription factor, the Oct-1/HCF/VP16 complex activates specifically only HSV IE genes.

One aspect of the present invention takes advantage of this sophisticated regulatory process and provides for the blocking of the HSV replicative cycle. Our invention provides for inhibiting IE gene expression and specifically by targeting TAATGARAT with nucleic acid binding polypeptides, for example, recombinant Zn finger transcription factors. Direct targeting of the genes expressed at the beginning of viral replicative cycle increases chances of inhibiting viral infection before HSV genome replicates.

In a particular embodiment of the invention, we disclose a polypeptide capable of binding to a nucleic acid comprising a sequence present in the Herpes Simplex Virus 1 (HSV-1) promoter, in which the polypeptide comprises three zinc fingers F1, F2 and F3, at least one of the amino acids at positions −1, 3 and 6 of F1, −1, 3 and 6 of F2 and −1, 3 and 6 of F3 are selected from amino acids specified in the following table:

| F1: amino acid | |
|---|---|
| −1 | R, T |
| 3 | E, N |
| 6 | R |
| F2 | |
| −1 | R, Q |
| 3 | H |
| 6 | T, E |
| F3 | |
| −1 | T, Q |
| 3 | N |
| 6 | K, T |

In a further embodiment, the polypeptide comprises three zinc fingers F1, F2 and F3, at least one of the amino acids at positions −1, 1, 2, 3, 4, 5 and 6 of F1, −1, 1, 2, 3, 4, 5 and 6 of F2 and −1, 1, 2, 3, 4, 5 and 6 of F3 are selected from amino acids specified in the following table:

| F1: amino acid | |
|---|---|
| −1 | R, T |
| 1 | S, R |
| 2 | D, T |
| 3 | E, N |
| 4 | L |
| 5 | T |
| 6 | R |
| F2 | |
| −1 | R, Q |
| 1 | S, D |
| 2 | D, A |
| 3 | H |
| 4 | L |
| 5 | S |
| 6 | T, E |
| F3 | |
| −1 | T, Q |
| 1 | N, S |
| 2 | S, N, A |
| 3 | N |
| 4 | R, N |
| 5 | I, K |
| 6 | K, T |

Preferably, each of the amino acids at the numbered positions are selected from amino acids specified in the table. Where reference is made to positions −1, 1, 2, 3, 4, 5 or 6 in the above, these positions are to be understood as referring to the relevant amino acid positions in Formulas A′ or B. Preferably, the positions are to be understood to refer to Formula A′. The zinc finger will of course further comprise backbone residues are defined in the relevant Formula but some variability will be allowed in the choice of these backbone residues.

In a preferred embodiment of the invention, a nucleic acid binding polypeptide capable of binding a herpes virus nucleotide sequence comprises one or more of the following sequences:

| SEQ ID | | |
|---|---|---|
| ID NO: | Sequence | Name |
| | X₀₋₂ C X₁₋₅ C X₂₋₇ R S D E L T R H X₃₋₆ ^{H}/_{C} | 4/3 F1 |
| | X₀₋₂ C X₁₋₅ C X₂₋₇ R S D H L S T H X₃₋₆ ^{H}/_{C} | 4/3 F2 |
| | X₀₋₂ C X₁₋₅ C X₂₋₇ T N S N R I K H X₃₋₆ ^{H}/_{C} | 4/3 F3 |
| | X₀₋₂ C X₁₋₅ C X₂₋₇ R S D E L T R H X₃₋₆ ^{H}/_{C} | 4A F1 |
| | X₀₋₂ C X₁₋₅ C X₂₋₇ R S D H L S E H X₃₋₆ ^{H}/_{C} | 4A F2 |
| | X₀₋₂ C X₁₋₅ C X₂₋₇ T N N N R K K H X₃₋₆ ^{H}/_{C} | 4A F3 |
| | X₀₋₂ C X₁₋₅ C X₂₋₇ T R T N L T R H X₃₋₆ ^{H}/_{C} | 7N F1 |
| | X₀₋₂ C X₁₋₅ C X₂₋₇ Q D A H L S T H X₃₋₆ ^{H}/_{C} | 7N F2 |
| | X₀₋₂ C X₁₋₅ C X₂₋₇ Q S A N R K T H X₃₋₆ ^{H}/_{C} | 7N F3 |
| | X₀₋₂ C X₁₋₅ C X₂₋₇ R S D E L T R H X₃₋₆ ^{H}/_{C} | 4/3 |
| | - linker - X₀₋₂ C X₁₋₅ C X₂₋₇ R S D H L S T | |
| | H X₃₋₆ ^{H}/_{C} - linker - X₀₋₂ C X₁₋₅ C X₂₋₇ T N | |
| | S N R I K H X₃₋₆ ^{H}/_{C} | |
| | X₀₋₂ C X₁₋₅ C X₂₋₇ T R T N L T R H X₃₋₆ ^{H}/_{C} | 4A |
| | - linker - X₀₋₂ C X₁₋₅ C X₂₋₇ R S D H L S E | |
| | H X₃₋₆ ^{H}/_{C} - linker - X₀₋₂ C X₁₋₅ C X₂₋₇ T N | |
| | N N R K K H X₃₋₆ ^{H}/_{C} | |
| | X₀₋₂ C X₁₋₅ C X₂₋₇ T R T N L T R H X₃₋₆ ^{H}/_{C} | 7N |
| | - linker - X₀₋₂ C X₁₋₅ C X₂₋₇ Q D A H L S T | |
| | H X₃₋₆ ^{H}/_{C} - linker - X₀₋₂ C X₁₋₅ C X₂₋₇ Q S | |
| | A N R K T H X₃₋₆ ^{H}/_{C} | |
| | MAEERPYACPVESCDRRFSRSDELTRHIRIHTGQKPFQ | 4/3 |
| | CRICMRNFSRSDHLSTHIRTHTGEKPFACDICGRKFAT | |
| | NSNRIKHTKIHLRQKDAA | |
| | MAEERPYACPVESCDRRFSRSDELTRHIRIHTGQKPFQ | 4A |
| | CRICMRNFSRSDHLSEHIRTHTGEKPFACDICGRKFAT | |
| | NNNRKKHTKIHLRQKDAA | |
| | MAEERPYACPVESCDRRFSTRTNLTRHIRIHTGQKPFQ | 7N |
| | CRICMRNFSQDAHLSTHIRTHTGEKPFACDICGRKFAQ | |
| | SANRKTHTKIHLRQKDAA | |
| | MAEERPYACPVESCDRRFSTRTNLTRHIRIHTGQKPFQ | 6F6 |
| | CRICMRNFSQDAHLSTHIRTHTGEKPFACDICGRKFAQ | |
| | SANRKTHTKIHLRQKDGERPYACPVESCDRRFSRSDEL | |
| | TRHIRIHTGQKPFQCRICMRNFSRSDHLSTHIRTHTGE | |
| | KPFACDICGRKFATNSNRIKHTKIHLRQKDAARNSTTL | |
| | D | |
| | MAEERPYACPVESCDRRFSTRTNLTRHIRIHTGQKPFQ | 6F6-KOX |
| | CRICMRNFSQDAHLSTHIRTHTGEKPFACDICGRKFAQ | |
| | SANRKTHTKIHLRQKDGERPYACPVESCDRRFSRSDEL | |
| | TRHIRIHTGQKPFQCRICMRNFSRSDHLSTHIRTHTGE | |
| | KPFACDICGRKFATNSNRIKHTKIHLRQKDAARNSGPK | |
| | KRKVDGGGALSPQHSAVTQGSIIKNKEGMDAKSLTAWS | |
| | RTLVTFKDVFVDFTREEWKLLDTAQQIVYRNVMLENYK | |
| | NLVSLGYQLTKPDVILRLEKGEEPWLVEREIHQETHPD | |
| | SETAFEIKSSVEQKLISEDL | |

Variants and Derivatives

The nucleic acid binding polypeptide molecule as provided by the present invention includes splice variants encoded by mRNA generated by alternative splicing of a primary transcript, amino acid mutants, glycosylation variants and other covalent derivatives of said molecule which retain the physiological and/or physical properties of said molecule, such as its nucleic acid binding activity. Exemplary derivatives include molecules wherein the protein of the invention is covalently modified by substitution, chemical, enzymatic, or other appropriate means with a moiety other than a naturally occurring amino acid. Such a moiety may be a detectable moiety such as an enzyme or a radioisotope, or may be a molecule capable of facilitating crossing of cell membrane(s) etc.

Derivatives can be fragments of the nucleic acid binding molecule. Fragments of said molecule comprise individual domains thereof, as well as smaller polypeptides derived from the domains. Preferably, smaller polypeptides derived from the molecule according to the invention define a single epitope which is characteristic of said molecule. Fragments may in theory be almost any size, as long as they retain one characteristic of the nucleic acid binding molecule. Preferably, fragments may be at least 3 amino acids and in length.

Derivatives of the nucleic acid binding molecule also comprise mutants thereof, which may contain amino acid deletions, additions or substitutions, subject to the requirement to maintain at least one feature characteristic of said molecule. Thus, conservative amino acid substitutions may be made substantially without altering the nature of the molecule, as may truncations from the N- or C-terminal ends, or the corresponding 5′- or 3′-ends of a nucleic acid encoding it. Deletions or substitutions may moreover be made to the fragments of the molecule comprised by the invention. Nucleic acid binding molecule mutants may be produced from a DNA encoding a nucleic acid binding protein which has been subjected to in vitro mutagenesis resulting e.g. in an addition, exchange and/or deletion of one or more amino acids. For example, substitutional, deletional or insertional variants of the molecule can be prepared by recombinant methods and screened for nucleic acid binding activity as described herein.

The fragments, mutants and other derivatives of the polypeptide nucleic acid binding molecule preferably retain substantial homology with said molecule. As used herein, “homology” means that the two entities share sufficient characteristics for the skilled person to determine that they are similar in origin and/or function Preferably, homology is used to refer to sequence identity. Thus, the derivatives of the molecule preferably retain substantial sequence identity with the sequence of said molecule. Examples of such sequences are presented as SEQ ID Nos 1 to 8. “Substantial homology”, where homology indicates sequence identity, means more than 75% sequence identity and most preferably a sequence identity of 90% or more. Amino acid sequence identity may be assessed by any suitable means, including the BLAST comparison technique which is well known in the art, and is described in Ausubel et al., Short Protocols in Molecular Biology (1999) 4^{th} Ed, John Wiley & Sons, Inc.

Mutations

Mutations may be performed by any method known to those of skill in the art. Preferred, however, is site-directed mutagenesis of a nucleic acid sequence encoding the protein of interest. A number of methods for site-directed mutagenesis are known in the art, from methods employing single-stranded phage such as M13 to PCR-based techniques (see “PCR Protocols: A guide to methods and applications”, M. A. Innis, D. H. Gelfand, J. J. Sninsky, T. J. White (eds.). Academic Press, New York, 1990). Preferably, the commercially available Altered Site II Mutagenesis System (Promega) may be employed, according to the directions given by the manufacturer.

Screening of the proteins produced by mutant genes is preferably performed by expressing the genes and assaying the binding ability of the protein product A simple and advantageously rapid method by which this may be accomplished is by phage display, in which the mutant polypeptides are expressed as fusion proteins with the coat proteins of filamentous bacteriophage, such as the minor coat protein pII of bacteriophage ml 3 or gene III of bacteriophage Fd, and displayed on the capsid of bacteriophage transformed with the mutant genes. The target nucleic acid sequence is used as a probe to bind directly to the protein on the phage surface and select the phage possessing advantageous mutants, by affinity purification. The phage are then amplified by passage through a bacterial host, and subjected to further rounds of selection and amplification in order to enrich the mutant pool for the desired phage and eventually isolate the preferred clone(s). Detailed methodology for phage display is known in the art and set forth, for example, in U.S. Pat. No. 5,223,409; Choo and Klug, (1995) Current Opinions in Biotechnology 6:431436; Smith, (1985) Science 228:1315-1317; and McCafferty et al., (1990) Nature 348:552-554; all incorporated herein by reference. Vector systems and kits for phage display are available commercially, for example from Pharmacia.

The present invention allows the production of what are essentially artificial nucleic acid binding proteins. In these proteins, artificial analogues of amino acids may be used, to impart the proteins with desired properties or for other reasons. Thus, the term “amino acid”, particularly in the context where “any amino acid” is referred to, means any sort of natural or artificial amino acid or amino acid analogue that may be employed in protein construction according to methods known in the art. Moreover, any specific amino acid referred to herein may be replaced by a functional analogue thereof, particularly an artificial functional analogue. The nomenclature used herein therefore specifically comprises within its scope functional analogues of the defined amino acids.

The polypeptides which comprise the libraries according to the invention may comprise zinc finger polypeptides. In other words, they comprise a Cys2-His2 zinc finger motif.

Molecules according to the invention may advantageously comprise multiple zinc finger motifs. For example, molecules according to the invention may comprise any number of motifs, such as three zinc finger motifs, or may comprise four or five such motifs, or may comprise six zinc finger motifs, or even more. Advantageously, molecules according to the invention may comprise zinc finger motifs in multiples of three, such as three, six, nine or even more zinc finger motifs. Preferably, molecules according to the invention may comprise about three to about six zinc finger motifs.

Vectors

The nucleic acid encoding the nucleic acid binding protein according to the invention can be incorporated into vectors for further manipulation. As used herein, vector (or plasmid) refers to discrete elements that are used to introduce heterologous nucleic acid into cells for either expression or replication thereof. Selection and use of such vehicles are well within the skill of the person of ordinary skill in the art. Many vectors are available, and selection of appropriate vector will depend on the intended use of the vector, i.e. whether it is to be used for DNA amplification or for nucleic acid expression, the size of the DNA to be inserted into the vector, and the host cell to be transformed with the vector. Each vector contains various components depending on its function. (amplification of DNA or expression of DNA) and the host cell for which it is compatible. The vector components generally include, but are not limited to, one or more of the following: an origin of replication, one or more marker genes, an enhancer element, a promoter, a transcription termination sequence and a signal sequence.

Both expression and cloning vectors generally contain nucleic acid sequence that enable the vector to replicate in one or more selected host cells. Typically in cloning vectors, this sequence is one that enables the vector to replicate independently of the host chromosomal DNA, and includes origins of replication or autonomously replicating sequences. Such sequences are well known for a variety of bacteria, yeast and viruses. The origin of replication from the plasmid pBR322 is suitable for most Gram-negative bacteria, the 2 μ plasmid origin is suitable for yeast, and various viral origins (e.g. SV 40, polyoma, adenovirus) are useful for cloning vectors in mammalian cells. Generally, the origin of replication component is not needed for mammalian expression vectors unless these are used in mammalian cells competent for high level DNA replication, such as COS cells.

Most expression vectors are shuttle vectors, i.e. they are capable of replication in at least one class of organisms but can be transfected into another class of organisms for expression. For example, a vector is cloned in E. coli and then the same vector is transfected into yeast or mammalian cells even though it is not capable of replicating independently of the host cell chromosome. DNA may also be replicated by insertion into the host genome. However, the recovery of genomic DNA encoding the nucleic acid binding protein is more complex than that of exogenously replicated vector because restriction enzyme digestion is required to excise nucleic acid binding protein DNA. DNA can be amplified by PCR and be directly transfected into the host cells without any replication component.

Selectable Markers

Advantageously, an expression and cloning vector may contain a selection gene also referred to as selectable marker. This gene encodes a protein necessary for the survival or growth of transformed host cells grown in a selective culture medium. Host cells not transformed with the vector containing the selection gene will not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available from complex media.

As to a selective gene marker appropriate for yeast, any marker gene can be used which facilitates the selection for transformants due to the phenotypic expression of the marker gene. Suitable markers for yeast are, for example, those conferring resistance to antibiotics G418, hygromycin or bleomycin, or provide for prototrophy in an auxotrophic yeast mutant, for example the URA3, LEU2, LYS2, TRP1, or HIS3 gene.

Since the replication of vectors is conveniently done in E. coli, an E. coli genetic marker and an E. coli origin of replication are advantageously included. These can be obtained from E. coli plasmids, such as pBR322, Bluescript© vector or a pUC plasmid, e.g. pUC18 or pUC19, which contain both E. coli replication origin and E. coli genetic marker conferring resistance to antibiotics, such as ampicillin.

Suitable selectable markers for mammalian cells are those that enable the identification of cells competent to take up nucleic acid binding protein nucleic acid, such as dihydrofolate reductase (DHFR, methotrexate resistance), thymidine kinase, or genes conferring resistance to G418 or hygromycim. The mammalian cell transformants are placed under selection pressure which only those transformants which have taken up and are expressing the marker are uniquely adapted to survive. In the case of a DHFR or glutamine synthase (GS) marker, selection pressure can be imposed by culturing the transformants under conditions in which the pressure is progressively increased, thereby leading to amplification (at its chromosomal integration site) of both the selection gene and the linked DNA that encodes the nucleic acid binding protein. Amplification is the process by which genes in greater demand for the production of a protein critical for growth, together with closely associated genes which may encode a desired protein, are reiterated in tandem within the chromosomes of recombinant cells. Increased quantities of desired protein are usually synthesised from thus amplified DNA.

Expression

Expression and cloning vectors usually contain a promoter that is recognised by the host organism and is operably linked to nucleic acid binding protein encoding nucleic acid. Such a promoter may be inducible or constitutive. The promoters are operably linked to DNA encoding the nucleic acid binding protein by removing the promoter from the source DNA by restriction enzyme digestion and inserting the isolated promoter sequence into the vector. Both the native nucleic acid binding protein promoter sequence and many heterologous promoters may be used to direct amplification and/or expression of nucleic acid binding protein encoding DNA.

Promoters suitable for use with prokaryotic hosts include, for example, the β-lactamase and lactose promoter systems, alkaline phosphatase, the tryptophan (Trp) promoter system and hybrid promoters such as the tac promoter. Their nucleotide sequences have been published, thereby enabling the skilled worker operably to ligate them to DNA encoding nucleic acid binding protein, using linkers or adapters to supply any required restriction sites. Promoters for use in bacterial systems will also generally contain a Shine-Delgarno sequence operably linked to the DNA encoding the nucleic acid binding protein.

Preferred expression vectors are bacterial expression vectors which comprise a promoter of a bacteriophage such as phagex or T7 which is capable of functioning in the bacteria In one of the most widely used expression systems, the nucleic acid encoding the fusion protein may be transcribed from the vector by T7 RNA polymerase (Studier et al, Methods in Enzymol. 185; 60-89, 1990). In the E. coli BL21(DE3) host strain, used in conjunction with pET vectors, the T7 RNA polymerase is produced from the α-lysogen DE3 in the host bacterium, and its expression is under the control of the IPTG inducible lac UV5 promoter. This system has been employed successfully for over-production of many proteins. Alternatively the polymerase gene may be introduced on a lambda phage by infection with an int-phage such as the CE6 phage which is commercially available (Novagen, Madison, USA), other vectors include vectors containing the lambda PL promoter such as PLEX (Invitrogen, NL), vectors containing the trc promoters such as pTrcH is XpressTm (Invitrogen) or pTrc99 (Pharmacia Biotech, SE) or vectors containing the tac promoter such as pKK223-3 (Pharmacia Biotech) or PMAL (New England Biolabs, MA, USA).

Moreover, the nucleic acid binding protein gene according to the invention preferably includes a secretion sequence in order to facilitate secretion of the polypeptide from bacterial hosts, such that it will be produced as a soluble native peptide rather than in an inclusion body. The peptide may be recovered from the bacterial periplasmic space, or the culture medium, as appropriate. A “leader” peptide may be added to the N-terminal finger. Preferably, the leader peptide is MAEEKP.

Suitable promoting sequences for use with yeast hosts may be regulated or constitutive and are preferably derived from a highly expressed yeast gene, especially a Saccharomyces cerevisiae gene. Thus, the promoter of the TRP1 gene, the ADHI or ADHII gene, the acid phosphatase (PH05) gene, a promoter of the yeast mating pheromone genes coding for the a- or α-factor or a promoter derived from a gene encoding a glycolytic enzyme such as the promoter of the enolase, glyceraldehyde-3-phosphate dehydrogenase (GAP), 3-phospho glycerate kinase (PGK), hexokinase, pyruvate decarboxylase, phosphofructokinase, glucose-6-phosphate isomerase, 3-phosphoglycerate mutase, pyruvate kinase, triose phosphate isomerase, phosphoglucose isomerase or glucokinase genes, or a promoter from the TATA binding protein (TBP) gene can be used. Furthermore, it is possible to use hybrid promoters comprising upstream activation sequences (UAS) of one yeast gene and downstream promoter elements including a functional TATA box of another yeast gene, for example a hybrid promoter including the UAS(s) of the yeast PH05 gene and downstream promoter elements including a functional TATA box of the yeast GAP gene (PH05-GAP hybrid promoter). A suitable constitutive PH05 promoter is e.g. a shortened acid phosphatase PH05 promoter devoid of the upstream regulatory elements (UAS) such as the PH05 (−173) promoter element starting at nucleotide −173 and ending at nucleotide −9 of the PH05 gene.

Nucleic acid binding protein gene transcription from vectors in mammalian hosts may be controlled by promoters derived from the genomes of viruses such as polyoma virus, adenovirus, fowlpox virus, bovine papilloma virus, avian sarcoma virus, cytomegalovirus (CMV), a retrovirus and Simian Virus 40 (SV40), from heterologous mammalian promoters such as the actin promoter or a very strong promoter, e.g. a ribosomal protein promoter, and from the promoter normally associated with nucleic acid binding protein sequence, provided such promoters are compatible with the host cell systems.

Transcription of a DNA encoding nucleic acid binding protein by higher eukaryotes may be increased by inserting an enhancer sequence into the vector. Enhancers are relatively orientation and position independent. Many enhancer sequences are known from mammalian genes (e.g. elastase and globin). However, typically one will employ an enhancer from a eukaryotic cell virus. Examples include the SV40 enhancer on the late side of the replication origin (bp 100-270) and the CMV early promoter enhancer. The enhancer may be spliced into the vector at a position 5′ or 3′ to nucleic acid binding protein DNA, but is preferably located at a site 5′ from the promoter.

Advantageously, a eukaryotic expression vector encoding a nucleic acid binding protein according to the invention may comprise a locus control region (LCR). LCRs are capable of directing high-level integration site independent expression of transgenes integrated into host cell chromatin, which is of importance especially where the nucleic acid binding protein gene is to be expressed in the context of a permanently-transfected eukaryotic cell line in which chromosomal integration of the vector has occurred, or in transgenic animals.

Eukaryotic vectors may also contain sequences necessary for the termination of transcription and for stabilising the mRNA. Such sequences are commonly available from the 5′ and 3′ untranslated regions of eukaryotic or viral DNAs or cDNAs. These regions contain nucleotide segments transcribed as polyadenylated fragments in the untranslated portion of the mRNA encoding nucleic acid binding protein.

An expression vector includes any vector capable of expressing nucleic acid binding protein nucleic acids that are operatively Linked with regulatory sequences, such as promoter regions, that are capable of expression-of such DNAs. Thus, an expression vector refers to a recombinant DNA or RNA construct, such as a plasmid, a phage, recombinant virus or other vector, that upon introduction into an appropriate host cell, results in expression of the cloned DNA. Appropriate expression vectors are well known to those with ordinary skill in the art and include those that are replicable in eukaryotic and/or prokaryotic cells and those that remain episomal or those which integrate into the host cell genome. For example, DNAs encoding nucleic acid binding protein may be inserted into a vector suitable for expression of cDNAs in mammalian cells, e.g. a CMV enhancer-based vector such as pEVRF (Matthias, et al., (1989) NAR 17, 6418).

Particularly useful for practising the present invention are expression vectors that provide for the transient expression of DNA encoding nucleic acid binding protein in mammalian cells. Transient expression usually involves the use of an expression vector that is able to replicate efficiently in a host cell, such that the host cell accumulates many copies of the expression vector, and, in turn, synthesises high levels of nucleic acid binding protein. For the purposes of the present invention, transient expression systems are useful e.g. for identifying nucleic acid binding protein mutants, to identify potential phosphorylation sites, or to characterise functional domains of the protein.

Construction of vectors according to the invention employs conventional ligation techniques. Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing nucleic acid binding protein expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe which may be based on a sequence provided herein. Those skilled in the art will readily envisage how these methods may be modified, if desired.

In accordance with another embodiment of the present invention, there are provided cells containing the above-described nucleic acids. Such host cells such as prokaryote, yeast and higher eukaryote cells may be used for replicating DNA and producing the nucleic acid binding protein. Suitable prokaryotes include eubacteria, such as Gram-negative or Gram-positive organisms, such as E. coli, e.g. E. coli K-12 strains, DH5a and HB101, or Bacilli. Further hosts suitable for the nucleic acid binding protein encoding vectors include eukaryotic microbes such as filamentous fungi or yeast, e.g. Saccharomyces cerevisiae; Higher eukaryotic cells include insect and vertebrate cells, particularly mammalian cells including-human cells or nucleated cells from other multicellular organisms. In recent years propagation of vertebrate cells in culture (tissue culture) has become a routine procedure. Examples of useful mammalian host cell lines are epithelial or fibroblastic cell lines such as Chinese hamster ovary (CHO) cells, NIH 3T3 cells, HeLa cells or 293T cells. The host cells referred to in this disclosure comprise cells in in vitro culture as well as cells that are within a host animal.

DNA may be stably incorporated into cells or may be transiently expressed using methods known in the art. Stably transfected mammalian cells may be prepared by transfecting cells with an expression vector having a selectable marker gene, and growing the transfected cells under conditions selective for cells expressing the marker gene. To prepare transient transfectants, mammalian cells are transfected with a reporter gene to monitor transfection efficiency.

To produce such stably or transiently transfected cells, the cells should be transfected with a sufficient amount of the nucleic acid binding protein-encoding nucleic acid to form the nucleic acid binding protein. The precise amounts of DNA encoding the nucleic acid binding protein may be empirically determined and optimised for a particular cell and assay.

Host cells are transfected or, preferably, transformed with the above-captioned expression or cloning vectors of this invention and cultured in conventional nutrient media modified as appropriate for inducing promoters, selecting transformants, or amplifying the genes encoding the desired sequences. Heterologous DNA may be introduced into host cells by any method known in the art, such as transfection with a vector encoding a heterologous DNA by the calcium phosphate coprecipitation technique or by electroporation. Numerous methods of transfection are known to the skilled worker in the field. Successful transfection is generally recognised when any indication of the operation of this vector occurs in the host cell. Transformation is achieved using standard techniques appropriate to the particular host cells used.

Incorporation of cloned DNA into a suitable expression vector, transfection of eukaryotic cells with a plasmid vector or a combination of plasmid vectors, each encoding one or more distinct genes or with linear DNA, and selection of transfected cells are well known in the art (see, e.g. Sambrook et al. (1989) Molecular Cloning: A Laboratory Manual, Second Edition, Cold Spring Harbor Laboratory Press).

Transfected or transformed cells are cultured using media and culturing methods known in the art, preferably under conditions, whereby the nucleic acid binding protein encoded by the DNA is expressed. The composition of suitable media is known to those in the art, so that they can be readily prepared. Suitable culturing media are also commercially available.

Nucleic acid binding molecules according to the invention may be employed in a wide variety of applications, including diagnostics and as research tools. Advantageously, they may be employed as diagnostic tools for identifying the presence of nucleic acid molecules in a complex mixture.

Preferred molecules according to the invention have gene-specific DNA binding activity. These may be constructed by the engineering of DNA-binding polypeptide domains with given DNA sequence-specificity, to target the appropriate gene(s).

Given the speed and convenience with which a great number of selections can be performed in parallel using the bipartite library strategy, we believe that the system is of great utility. The ‘bipartite’ system is a most time- and cost-effective general method of engineering zinc fingers by phage display.

Described herein is a rapid and convenient method that can be used to design zinc finger proteins against an unlimited set of DNA binding sites. This is based on a pair of pre-made zinc finger phage display libraries, which are used in parallel to select two DNA-binding domains that each recognise given 5 bp sequences, and whose products are recombined to produce a single protein that recognises a composite (10 bp) site of predefined sequence. Engineering using this system can be completed in less than two weeks and yields polypeptide molecules that bind sequence-specifically to DNA with K_{d}s in the nanomolar range. Library selection is therefore suitable for production of zinc fingers capable of binding to sequences within viral promoters, and may be augmented by rational or rule-based design (described elsewhere in this document). The present invention in one aspect thus relates to polypeptide molecules selected and/or designed to bind various regions of the human immunodeficiency virus 1 (HIV-1) promoter; for example eight different such molecules are described herein. Other polypeptides are capable of binding regions of an HSV promoter, for example, an IE promoter comprising a TAATGARAT motif. Our methods enable the production of polypeptides capable of binding to any viral promoter, by identification of a motif or sequence within that promoter, and selection of one or more zinc fingers (or other nucleic acid binding polypeptides) which bind to that sequence or motif.

As used herein, the term ‘region’ may mean part, segment, locus, area, fragment, motif, domain, section, site or similar part of said promoter, and may even include the promoter in its entirety. Thus, the phrase ‘region of the/a . . . promoter’ includes segment(s), fragments etc. of the promoter, and may include the whole promoter, or motifs therein such as transcription factor binding site(s), or other such parts thereof.

Presented herein is a novel zinc finger engineering strategy which (i) yields zinc finger polymers that bind DNA specifically, with good affinity, and without significant sequence restrictions on the generation of such polymer molecules, (ii) can be executed relatively rapidly, and (iii) can be easily adapted to a high-throughput automated format. This strategy is based on recent advances in our understanding of zinc finger function, particularly the phenomenon of synergistic DNA recognition by adjacent zinc fingers (11, 18), in combination with certain technical advances in zinc finger library design as discussed herein. The invention thus relates to the construction of a zinc finger library according to the new strategy disclosed herein. This and other aspects of the present invention are demonstrated by selecting a number of DNA-binding domains that specifically recognise the promoter region (LTR) of HIV-1, as well as selecting a number of nucleic acid binding domains which are capable of recognising an Immediate Early promoter of HSV.

It should be noted that it is possible for the recombinant proteins of the present invention to feature idiosyncratic combinations of amino acids that would not necessarily have been predicted by a recognition code. This is particularly true of the combinations of amino acids that are responsible for the inter-finger synergy that allows any base-pair to be specified at the interface of zinc finger DNA subsites (11). However, we note that the zinc fingers produced by the methods described in the Examples on the whole comply with the recognition code described above.

Zinc finger domains may be made by methods described and/or referred to herein. For example, said zinc finger DNA binding domains may be made as discussed in the examples, or as described in one or more of WO96/06166, WO98/53058, WO98/53057, or WO/98/53060.

The ‘Bipartite’ Library Strategy

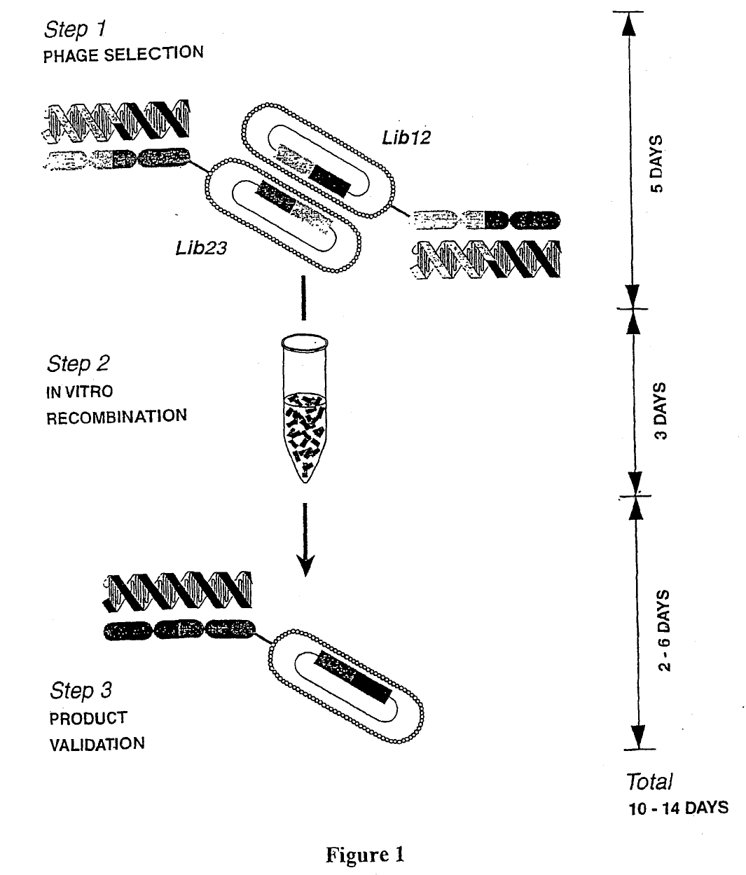

We have devised a ‘bipartite-complementary’ system for the construction of DNA-binding domains by phage display (FIG. 1). This system comprises two master libraries, Lib12 and Lib23, each of which encodes variants of a three-finger DNA-binding domain based on that of the transcription factor Zif268 (6, 19). The two libraries are complementary because Lib12 contains randomisations in all the base-contacting positions of F1 and certain base-contacting positions of F2, while Lib23 contains randomisations in the remaining base-contacting positions of F2 and all the base-contacting positions of F3 (FIG. 2a). The non-randomised DNA-contacting residues carry the nucleotide specificity of the parental Zif268 DNA-binding domain.

The design of the bipartite system features at least two modifications to the conventional zinc finger engineering strategies. As described above, each library contains members that are randomised in the α-helical DNA-contacting residues from more than one zinc finger. We have shown that the simultaneous randomisation of positions from adjacent fingers results in selected zinc finger pairs that can achieve comprehensive DNA recognition, i.e. bind DNA without significant sequence limitations.

The proteins produced by these libraries are therefore not limited to binding DNA sequences of the form GNNGNN . . . , as is the case with many prior art libraries (eg. 9, 13, 20). Furthermore, the repertoire of randomisations does not encode all 20 amino acids, rather representing only those residues that most frequently function in sequence-specific DNA binding from the respective α-helical positions (FIG. 2b). Excluding the residues that do not frequently function in DNA recognition advantageously helps to reduce the library size and/or the ‘noise’ associated with non-specific binding members of the library.

A brief outline of the bipartite strategy follows; it will be appreciated that the protocol does not need to be followed rigidly, and may be varied to the same end:

Phage selections from the two master libraries (Lib12 and Lib23) are performed using the generic DNA sequence 3′-HIJKLMGGCG-5′ for Lib12, and 3′-GCGGMNOPQ-5′ for Lib23, where the underlined bases are bound by the wild-type portion of the DNA-binding domain and each of the other letters represents any given nucleotide (FIG. 2a). The conserved nucleotides of the Zif268 binding site serve to fix the register of the interaction by binding to the conserved portion of the Zif268 DNA-binding domain in each library. Since the two complementary libraries have thus been designed to bind DNA in the same register, the selected DNA-binding portions from each library may then spliced to produce a recombinant three-finger polymer that recognises the predetermined DNA sequence 3′-HIJKLMNOPQ-5′. This DNA does not contain any of the sites bound by fingers of Zif268, nor does it impose any other DNA sequence limitation.

In order to operate the bipartite strategy the two zinc finger libraries may be subjected to selection in parallel using the appropriate DNA sequences as described above. The genes of the selected zinc fingers are amplified (for example by PCR), cut using an appropriate restriction enzyme (for example, DdeI) and recombined randomly by re-ligation of the resulting cohesive termini. The enzyme DdeI cuts the gene of either library at the same position in the α-helix of F2, allowing for seamless joining of selected zinc finger portions. A further PCR step, performed with selective primers, may be used to specifically recover the desired zinc finger product(s) from the pool of recombinants (which contains a number of genes including wild-type Zif-268). The recombined DNA-binding domains may be again displayed on phage, to be used in further rounds of selection in order to identify the optimal zinc finger product and/or to be used in phage ELISA experiments to assess binding to the composite target DNA.

The bipartite selection strategy allows the recombination in vitro of the complementary portions of the two libraries, without the need for further purification steps. We take advantage of selective PCR, so as to amplify only the products of recombination. PCR with enzymes lacking 5′Θ3′ exonuclease activity cannot proceed if primers contain one or more 3′ mismatches against their template binding sites. The two complementary libraries may therefore be designed with unique sequences at their 5′ and 3′ termini, and the corresponding primers used to amplify any recombinants of the two libraries. Furthermore, the selection procedure is amenable to a microtitre plate format so that selections and most subsequent manipulations may be automated (e.g., be carried out using liquid handling robots).

Many of the steps of the engineering process using our bipartite protocol—bacterial growth, phage selection, colony picking, phage ELISA, PCR and cloning—may be automated using commercially available instruments. Microtitre plates, such as 96 or 384 well microtitre plates, may be used to carry out phage selections, ELISA reactions and PCR preparation on a liquid-handling robotic platform. A robotic arm shuttles the microtitre plates between a pipeting station, a plate hotel, a plate washer, a spectrophotometer, and a PCR block. A colony picking robot may be used to inoculate micro-cultures of bacteria in microtitre plates in order to provide monoclonal phage for ELISA. A robot may be used that interfaces with the spectrophotometer and which is capable of returning to the liquid culture archive in order to ‘cherry-pick’ particular clones that are suitable for recombination, or which should be archived. A bar-coding system may be used to keep track of the various plates used for phage selections, phage ELISAs or for archiving interesting clones.

The ability to carry out selective PCR implies that the protocol may even be adapted to selecting complementary library portions in the same tube or well. For example, both universal libraries may be co-screened in a single well, thereby increasing the efficiency of high throughput applications. The output of such combined selections may be monitored by any means, for example, by selective PCR, or by ELISA of samples of isolated clones, etc.

This strategy is further discussed elsewhere in this application, such as in the Examples section. For example, Examples 1, 2 and 3 describe the use of this strategy to isolate zinc finger polypeptides which bind sequences within the HIV-1 promoter with high affinity and specificity.

In a preferred embodiment, the nucleic acid binding molecules of the invention can be incorporated into an ELISA assay. For example, phage displaying the molecules of the invention can be used to detect the presence of the target nucleic acid, and visualised using enzyme-linked anti-phage antibodies. The sites at which molecules according to the invention bind the target nucleic acid molecule may be determined by methods known in the art for example using binding assays, footprinting, truncation or mutant analysis.

Disclosed herein is a novel strategy of engineering zinc finger DNA-binding domains by phage display which has distinct advantages over the existing methods (1, 2), resulting in an advance in our ability to select and/or produce DNA-binding proteins.

As described above, an advantage of the present method is that it can produce zinc fingers binding to diverse DNA sequences, while other methods yield proteins that require the presence of G nucleotide at every third base position (13, 20). This feature of the present invention is based upon an improvement of our understanding of the synergistic nature of zinc finger interactions, as discussed herein. Prior art techniques have been confined to small subsets of G-rich DNA sequences. The ability to bind a variety of DNA sequences enables targeting of any given promoter in the genome, and is an advantageous feature of at least one aspect of the present invention.

Another advantage of the methods of the present invention is the speed with which DNA-binding domains may be produced. The main reason for the relatively fast turnover is that our new system takes advantage of pre-made phage display libraries, rather than being based on recurring library construction (2) in order to assemble a zinc finger polymer. This in turn allows for parallel (compared to serial) selection of zinc fingers from phage display libraries, thus saving time beyond that required simply for cloning. Additionally, the selective PCR protocols allow recombination to be advantageously carried out in vitro using a mixed population of zinc finger phage as starting material, thereby circumventing cumbersome clone isolation, DNA preparation and gel purification procedures. It is envisaged that the methods of the present invention may be useful in high-throughput protein engineering, such as via automation using liquid handling robotic systems.

Nucleic acid binding molecules according to the invention may comprise tag sequences to facilitate studies and/or preparation of such molecules. Tag sequences may include flag-tag, myc-tag, 6his-tag or any other suitable tag known in the art.

Another advantage of the present invention is the ability to target nucleic acid sequences which comprise cis-acting elements. Examples of cis-acting elements include promoters, enhancers, repressors, transcription factor binding sites, initiators, and other such nucleic acid sequences. Molecules according to the invention may advantageously be targeted to bind at and/or adjacent and/or near to such cis-acting elements. Preferably, molecules according to the invention may be targeted to transcription factor binding sites. By directing or targeting the nucleic acid binding molecules of the invention to nucleic acid sequences in this manner, surprisingly high effects, such as repression effects, may be achieved. This is discussed further below. Such molecules may be advantageously targeted to bind at sites comprising all or part of, or adjacent to, transcription factor sites such as SP1 sites, NF-kB sites, or any other transcription factor binding sites. Preferably, such molecules are targeted to SPI sites.

Preferably, the DNA-binding domains described herein are highly effective in repressing gene expression from nucleic acid molecules to which they bind. More preferably, the DNA-binding domains described herein are highly effective in repressing gene expression from the HIV-1 promoter. In a highly preferred embodiment, said repression of gene expression involves the binding of said DNA-binding domains to one or more region(s) of the HIV-1 promoter comprising or adjacent to one or more SPI transcription factor binding site(s).

Advantageously, molecules according to the invention may be used in combination. Use in combination includes both fusion of molecules into a single polypeptide as well as use of two or more discrete polypeptide molecules in solution. We have surprisingly shown a synergistic effect of using molecules according to the invention in combination. This is discussed elsewhere in the application, such as in the Examples.

Modulation by Binding to Transcription Factor Binding Sites

As noted above, our invention provides for methods of modulation of transcription by targeting nucleic acid sequences by use of nucleic acid binding polypeptides. Such target nucleic acid sequences may be ones which that overlap with transcription factor binding sites.

In one configuration, the polypeptide binds to a nucleic acid sequence comprising a transcription factor binding site or a variant or part thereof. Alternatively, the polypeptide may bind to a nucleic acid sequence adjacent to a transcription factor binding site or a variant or part thereof Furthermore, the polypeptide may bind to more than one nucleic acid sequence, each nucleic acid sequence comprising or being adjacent to a transcription factor binding site or a variant or part thereof.

The nucleic acid sequences may be targeted by any of the zinc finger polypeptides disclosed here. Furthermore, we provide a method of modulating transcription of a nucleic acid molecule comprising contacting the nucleic acid molecule with two or more polypeptides as disclosed here.

The transcription factor binding site may be a binding site for a known transcription factor. The transcription factor may be an animal, preferably vertebrate, or plant transcription factor. Such transcription factors, and their putative or determined binding sites, including any consensus motifs, are known in the art, and may be found in (for example), the “Transcription Factor Database”, at http://www.hsc.virginia.edu/achs/molbio/databases/tfd_dat.html. Reference is also made to Nucleic Acids Res 21, 3117-8 (1993), Gene Transcription: A Practical Approach, 32145 (1993) and Nucleic Acids Res 24, 238-41 (1996). A list of transcription factors, together with their binding sites, is contained in the file “tfsites.dat”, is a composite of the datasets TFD (release 7.5) SITES dataset file, March 1996 and Transfac (release 2.5) SITES dataset selected entries, January 1996. The file “tfsites.dat” may be obtained using the GCG command “FETCH tfsites.dat”. Any of these binding sites may be targeted according to the invention. Preferred transcription factors include those comprising homeodomains. Specific transcription factors and sites include those for NF-kB (GGGAAATTCC), Sp1 (consensus sequence G/T-GGGCGG-G/A-G/A-CM Oct-1 (ATTTGCAT), p53, myC, myB, AP1 etc.

Gene Therapy

A further application of the zinc fingers disclosed here is in the field of gene therapy for prevention or treatment of diseases, conditions, syndromes, or the prevention or relief of any of their symptoms. Any of the zinc fingers disclosed here may therefore be introduced into suitable target for such gene therapy.

In particular, the introduction by gene therapy of HIV inhibitors in T cell lymphocytes may be used as an alternative to conventional drug therapy for HIV infection. Molecules which have been tested in pre-clinical studies or gene therapy clinical trial include transdominant mutants of HIV proteins, anti-sense RNA, ribozymes or intracellular antibodies against HIV proteins. Accordingly, the zinc finger polypeptides of the present invention may be introduced into cells as a means of preventing or treating diseases such as viral diseases.

The target cell for introduction of the zinc finger will be chosen according to the condition or disease to be treated or prevented. The choice of suitable target cells will be known in the art. For example, for the treatment or prevention of HIV infection, the optimal target cell population for such strategy may comprise CD4⁺ peripheral blood lymphocytes. Alternatively, pluripotent haematopoietic stem cell (HSC), from which all CD4⁺ peripheral blood lymphocytes differentiate, may also be used as target cells.

Zinc finger constructs may be introduced into the target cell by any suitable means, for example as nucleic acid based expression constructs. Plasmid and other expression constructs are described in detail elsewhere in this document. Virus based vectors (for example, viral expression constructs) may also be used advantageously to effect gene delivery into a target cell. The viral vector is essentially an engineered virus, and retains its ability to express the gene of interest as well as maintaining its ability to deliver this gene to target cells. Other expression vectors are known in the art, and may also be used. Thus, any suitable vector, preferably a viral based vector, may be used as a means of introducing the nucleic acid binding polypeptides of the invention into target cells.

Retroviral (oncoretrovirus or lentivirus) based vectors are particularly attractive for gene delivery as they integrate efficiently into the host chromosomal DNA, resulting in the stable transmission and expression of the transgene. Successful gene transfer into peripheral blood lymphocytes or haematopoietic repopulating cells may be achieved with conventional oncoretroviral vectors, for example, those based on the Moloney murine leukemia virus (MoMuLV). Efficient retroviral gene transfer with MoMuLV-based vector to T cells and hematopoietic repopulating cells may be achieved by using cytokine or/and antibody prestimulation, high titer pseudotyped retroviral vectors and co-localisation of retroviral particles and target cells.

Gene therapy clinical protocols used for successful transduction into peripheral blood lymphocytes from HIV-infected patients (Wong-Staal et al., Human Gene Therapy, 1998; Cooper et al., Human Gene Therapy, 1999) or haematopoietic repopulating cells (Cavazzana-Calvo et al., Science, 2000) are known in the art, and may for example be used for the clinical gene delivery of HIV-BA′-KOX protein to CD4⁺ T cells derived from HIV patients. Examples 11 and 12 below disclose protocols may be used for the transduction of zinc finger expression constructs into peripheral blood CD4⁺ T lymphocytes and CD34⁺ repopulating cells.

The vector which may be used may include vectors, for example, based on the LNL or derivative MoMuLV-based oncoretroviral vector encoding for HIV-BA′-KOX gene, as shown in the Examples. Alternatively a lentiviral or other vector could be used. Recombinant viral particles may be pseudotyped with amphotropic, feline endogenous retrovirus (RD114) envelope protein, Gibbon Ape Leukemia virus (GALV) envelope protein G protein of vesicular stomatitis virus (VSV-G) for successful infection of human cells.

Pharmaceuticals

Moreover, the invention provides therapeutic agents and methods of therapy involving use of nucleic acid binding proteins as described herein. In particular, the invention provides the use of polypeptide fusions comprising an integrase, such as a viral integrase, and a nucleic acid binding protein according to the invention to target nucleic acid sequences in vivo (Bushman, (1994) PNAS (USA) 91:9233-9237). In gene therapy applications, the method may be applied to the delivery of functional genes into defective genes, or the delivery of nonsense nucleic acid in order to disrupt undesired nucleic acid. Alternatively, genes may be delivered to known, repetitive stretches of nucleic acid, such as centromeres, together with an activating sequence such as an LCR. This would represent a route to the safe and predictable incorporation of nucleic acid into the genome.

In conventional therapeutic applications, nucleic acid binding proteins according to the invention may be used to specifically knock out cells having mutant vital proteins. For example, if cells with mutant ras are targeted, they will be destroyed because ras is essential to cellular survival. Alternatively, the action of transcription factors may be modulated, preferably reduced, by administering to the cell agents which bind to the binding site specific for the transcription factor. For example, the activity of HIV tat may be reduced by binding proteins specific for HIV TAR.

Moreover, binding proteins according to the invention may be coupled to toxic molecules, such as nucleases, which are capable of causing irreversible nucleic acid damage and cell death. Such agents are capable of selectively destroying cells which comprise a mutation in their endogenous nucleic acid.

Nucleic acid binding proteins and derivatives thereof as set forth above may also be applied to the treatment of infections and the like in the form of organism-specific antibiotic or antiviral drugs. In such applications, the binding proteins may be coupled to a nuclease or other nuclear toxin and targeted specifically to the nucleic acids of microorganisms.

The invention likewise relates to pharmaceutical preparations which contain the compounds according to the invention or pharmaceutically acceptable salts thereof as active ingredients, and to processes for their preparation.

The pharmaceutical preparations according to the invention which contain the compound according to the invention or pharmaceutically acceptable salts thereof are those for enteral, such as oral, furthermore rectal, and parenteral administration to (a) warm-blooded animal(s), the pharmacological active ingredient being present on its own or together with a pharmaceutically acceptable carrier. The daily dose of the active ingredient depends on the age and the individual condition and also on the manner of administration.

The novel pharmaceutical preparations contain, for example, from about 10% to about 80%, preferably from about 20% to about 60%, of the active ingredient. Pharmaceutical preparations according to the invention for enteral or parenteral administration are, for example, those in unit dose forms, such as sugar-coated tablets, tablets, capsules or suppositories, and furthermore ampoules. These are prepared in a manner known per se, for example by means of conventional mixing, granulating, sugar-coating, dissolving or lyophilising processes. Thus, pharmaceutical preparations for oral use can be obtained by combining the active ingredient with solid carriers, if desired granulating a mixture obtained, and processing the mixture or granules, if desired or necessary, after addition of suitable excipients to give tablets or sugar-coated tablet cores.

Suitable carriers are, in particular, fillers, such as sugars, for example lactose, sucrose, mannitol or sorbitol, cellulose preparations and/or calcium phosphates, for example tricalcium phosphate or calcium hydrogen phosphate, furthermore binders, such as starch paste, using, for example, corn, wheat, rice or potato starch, gelatin, tragacanth, methylcellulose and/or polyvinylpyrrolidone, if desired, disintegrants, such as the abovementioned starches, furthermore carboxymethyl starch, crosslinked polyvinylpyrrolidone, agar, alginic acid or a salt thereof, such as sodium alginate; auxiliaries are primarily glidants, flow-regulators and lubricants, for example silicic acid, talc, stearic acid or salts thereof, such as magnesium or calcium stearate, and/or polyethylene glycol. Sugar-coated tablet cores are provided with suitable coatings which, if desired, are resistant to gastric juice, using, inter alia, concentrated sugar solutions which, if desired, contain gum arabic, talc, polyvinylpyrrolidone, polyethylene glycol and/or titanium dioxide, coating solutions in suitable organic solvents or solvent mixtures or, for the preparation of gastric juice-resistant coatings, solutions of suitable cellulose preparations, such as acetylcellulose phthalate or hydroxypropylmethylcellulose phthalate. Colorants or pigments, for example to identify or to indicate different doses of active ingredient, may be added to the tablets or sugar-coated tablet coatings.

Other orally utilisable pharmaceutical preparations are hard gelatin capsules, and also soft closed capsules made of gelatin and a plasticiser, such as glycerol or sorbitol. The hard gelatin capsules may contain the active ingredient in the form of granules, for example in a mixture with fillers, such as lactose, binders, such as starches, and/or lubricants, such as talc or magnesium stearate, and, if desired, stabilisers. In soft capsules, the active ingredient is preferably dissolved or suspended in suitable liquids, such as fatty oils, paraffin oil or liquid polyethylene glycols, it also being possible to add stabilisers.

Suitable rectally utilisable pharmaceutical preparations are, for example, suppositories, which consist of a combination of the active ingredient with a suppository base. Suitable suppository bases are, for example, natural or synthetic triglycerides, paraffin hydrocarbons, polyethylene glycols or higher alkanols. Furthermore, gelatin rectal capsules which contain a combination of the active ingredient with a base substance may also be used. Suitable base substances are, for example, liquid triglycerides, polyethylene glycols or paraffin hydrocarbons. Suitable preparations for parenteral administration are primarily aqueous solutions of an active ingredient in water-soluble form, for example a water-soluble salt, and furthermore suspensions of the active ingredient, such as appropriate oily injection suspensions, using suitable lipophilic solvents or vehicles, such as fatty oils, for example sesame oil, or synthetic fatty acid esters, for example ethyl oleate or triglycerides, or aqueous injection suspensions which contain viscosity-increasing substances, for example sodium carboxymethylcellulose, sorbitol and/or dextran, and, if necessary, also stabilisers.

The dose of the active ingredient depends on the warm-blooded animal species, the age and the individual condition and on the manner of administration. In the normal case, an approximate daily dose of about 10 mg to about 250 mg is to be estimated in the case of oral administration for a patient weighing approximately 75 kg

### EXAMPLES

### Example 1

### Construction of Phage Display Libraries for Selection of DNA-Binding Domains

Zinc fingers capable of binding HIV nucleotide sequences are constructed using a ‘bipartite-complementary’ system as described above and illustrated in FIG. 1. This system comprises two master libraries, Lib12 and Lib23, each of which encodes variants of a three-finger DNA-binding domain based on that of the transcription factor Zif268 (6, 19), which are complementary as Lib12 contains randomisations in all the base-contacting positions of F1 and certain base-contacting positions of F2, while Lib23 contains randomisations in the remaining base-contacting positions of F2 and all the base-contacting positions of F3 (FIG. 2a). The non-randomised DNA-contacting residues carry the nucleotide specificity of the parental Zif268 DNA-binding domain.

The libraries are constructed by known techniques, briefly described here.

Gene inserts for phage libraries are constructed by end-to-end ligation of selectively randomised dsDNA ‘minicassettes’, made individually by annealing complementary template oligonucleotides. The resulting genes may then be amplified by PCR and code for zinc fingers in a suitable reading frame for cloning as fusions to the phage minor coat protein, pIII. Any suitable scaffold may be used, for example, the DNA-binding domain of the transcription factor Zif268, which contains three Cys₂-His₂ zinc fingers whose mode of binding is well understood.

In order to selectively randomise the α-helix of a zinc finger, the coding region is synthesised using DNA mini-cassettes, such that helical positions −1 through 4 are encoded by one cassette (minicassette 2), while positions 4 through 6 are encoded by another cassette (minicassette 3). These double stranded ‘cassettes’ are synthesised with complementary overhangs that anneal through the codon for the fourth α-helical residue, which is invariant. Each ‘cassette’ actually comprises a library of oligonucleotides synthesised with appropriate codon randomisations so as to code for a given subset of amino acids. The first cassette is a single sequence and codes for the invariant β-sheet region, while the second and third cassettes contain randomisations of the α-helix. Each of the ‘library mini-cassettes’ comprises numerous oligonucleotides created through a limited number of solid-phase syntheses: minicassette 2 requires oligonucleotides from 12 pairs of syntheses, while minicassette 3 requires oligonucleotides from three pairs of syntheses. Each oligonucleotide synthesis is designed to introduce a very limited variability into each cassette—the library complexity is increased by the use of oligonucleotides from multiple syntheses and by the combination of the two mini-cassettes.

Genes for the two zinc finger phage display libraries (Lib12 and Lib23) are assembled from synthetic DNA oligonucleotides by directional end-to-end ligation using short complementary DNA linkers as described above. In order to include only the amino acids shown in FIG. 2b, a large number of appropriately randomised oligonucleotides (each encoding a subset of a few amino acids) are used in combinations to assemble the gene cassettes. These are amplified by PCR, digested with SfiI and NotI endonucleases, and ligated into the phage vector Fd-Tet-SN (9). E. coli TGI cells are transformed with the recombinant vector by electroporation and plated onto TYE medium (1.5% (w/v) agar, 1% (w/v) Bactotryptone, 0.5% (w/v) Bactoyeast extract, 0.8% (w/v) NaCl) containing 15 μg/ml tetracycline. The theoretical library sizes of Lib12 and Lib23 are approx. 4.9×10⁶ and approx. 2.1×10⁶, respectively (FIG. 2b). Approximately twice these numbers of bacterial transformants are obtained for the respective libraries.

A detailed library construction protocol follows:

Single-stranded template oligonucleotides are phosphorylated in a kinase reaction prior to assembly (100 pmol of each oligonucleotide in 10 μl of 1×T4 kinase buffer, containing 1 mM DATP and 10 U T4 polynucleotide kinase, 37°, 1 hr). Complementary single-stranded template oligonucleotides are annealed pairwise to form double-stranded minicassettes: 100 pmol of each oligonucleotide (or, for smart randomisation, 100 pmol of each strand mixture) are mixed in 1×T4 ligase or kinase buffer, to a final DNA concentration of 10 pmol/μl. Annealing is by heating to 94° and then cooling slowly (˜1 hr) to room temperature. The resulting dsDNA minicassettes are combined and ligated by adding an equal volume of 1×T4 ligase buffer and 8 μl (3200 U) of T4 ligase per 100 μl (160, 20 hr).

Full-length genes are amplified by PCR from the ligation mixture with primers that introduce NotI and SfiI restriction sites for cloning into phage vector Fd-TET-SN. Thorough digestion with these endonucleases is essential for high-efficiency ligation into similarly prepared phage vector (200 U enzyme per 40 μg DNA, with 8 hr incubation in appropriate temperatures and buffers, adding enzymes in stages at 2-hr intervals). Typically, 1 μg of pure phage vector is ligated with a 5-fold excess of gene cassette insert (1×T4 ligase buffer, 3 μl T4 ligase, 30 μl total volume, 16°, 20 hr). Ligation reactions are prepared for electroporation by washing twice in an equal volume of chloroform and precipitating by adding 1/10 volume sodium acetate (pH 5.5) and 3 volumes of ethanol¹⁴. DNA pellets are washed with 70% ethanol and resuspended in sterile water to a final concentration of 200 ng/μl.

The phage library is cloned by electroporation of recombinant vector into a suitable strain of E. coli, such as TG1. Typically, 0.5 μg of recombinant phage vector can be used with 100 μl of electrocompetent cells¹⁵, yielding up to 106 library transformants (2 mm path cuvette, 2.5 kV, 25, 200 ohms). After pulsing, cells are immediately resuspended in 1 ml SOC and incubated without shaking (37°, 1 hr). Fd-TET-SN confers tetracycline resistance allowing positive selection of bacterial transformants by plating on 2×YT-agar plates, containing 15 μg/ml tetracycline (37°, 16 hr).

### Example 2

### Production of DNA-Binding Domains that Target the HIV-1 Promoter

Phage selections from the two master libraries described in Example 1 (Lib12 and Lib23) are performed using the generic DNA sequence 3′-HIJKLMGGCG-5′ for Lib12, and 3′-GCGGMNOPQ-5′ for Lib23, where the underlined bases are bound by the wild-type portion of the DNA-binding domain and each of the other letters represents any given nucleotide (FIG. 2a). A number of sites in the well-characterised promoter of HIV-1 are targeted.

In this example, the two zinc finger libraries (Lib12 and Lib23) are subjected to selection in parallel, the nucleotide sequences used (ie. HIJKL/MNOPQ) being from HIV-1 between positions −80 and +60 (see Table 1/FIG. 3).

Tetracycline resistant bacterial colonies are transferred to 2×TY liquid medium (16 g/litre Bactotryptone, 10 g/litre Bactoyeast extract, 5 g/litre NaCl) containing 50 μM ZnCl₂ and 15 μg/ml tetracycline, and cultured overnight at 30° C. in a shaking incubator. Cleared culture supernatant containing phage particles is obtained by centrifuging at 300 g for 5 minutes.

One picomole of biotinylated DNA target site is bound to streptavidin-coated tubes (Roche), in 50 μl PBS containing 50 μM ZnCl₂. Bacterial culture supernatant containing phage is diluted 1:10 in selection buffer (PBS containing-50 μM ZnCl, 2% (w/v) fat-free dried milk (Marvel), 1% (v/v) Tween, 20 mg/ml sonicated salmon sperm DNA), and 1 ml is applied to each tube. Binding reactions are incubated for 1 hour at 20° C., after which the tubes are emptied and washed 20 times with PBS containing 50 μM ZnCl₂, 2% (w/v) fat-free dried milk (Marvel) and 1% (v/v) Tween.

Retained phage are eluted in 0.1 M triethylamine and neutralised with an equal volume of 1 M Tris-HCl (pH 7.4). Logarithmic-phase E. coli TG1 are infected with eluted phage, and cultured overnight at 30° C. in 2×TY medium containing 50 μM ZnCl₂ and 15 μg/ml tetracycline, to amplify phage for further rounds of selection.

After 5 rounds of selection, E. coli TG1 infected with selected phage are plated and individual colonies are picked and cultured in liquid medium (20). Clones which recognise their target site are retained for subsequent recombination of the two complementary halves recovered from Lib12 and Lib23. A brief protocol follows:

The genes of the selected zinc fingers are amplified by PCR, cut using the restriction enzyme DdeI and recombined randomly by re-ligation of the resulting cohesive termini. The enzyme DdeI cuts the gene of either library at the same position in the α-helix of F2, allowing for seamless joining of selected zinc finger portions.

The zinc finger genes of the selected clones are recovered by PCR from phage template present in 1 μl eluate. PCR products are diluted in two volumes of DdeI buffer (NEBuffer 3; New England Biolabs, USA) and digested using 40 units DdeI per 100 μl. After heat inactivation of the restriction enzyme, the reaction is made up to T4 ligase buffer (New England Biolabs, USA) and 400 units T4 ligase are added to a 10 μl reaction, and incubated for 15 hours at 20° C.

A further PCR step, performed with selective primers, is used to specifically recover the desired zinc finger product(s) from the pool of recombinants (which contains a number of genes including wild-type Zif268) as follows.

Recombinants comprising the selected portions of Lib12 and Lib23 are amplified selectively by PCR from 1 μl of the ligation mixture, using primers corresponding to unique sequences in the N-terminus of Lib-12 and the C-terminus of Lib-23 (20 cycles of amplification with Taq polymerase). Recombinant DNA-binding domains are cloned into Fd-Tet-SN as described above.

The recombined DNA-binding domains are displayed on phage, and used in further rounds of selection in order to identify the optimal zinc finger product and/or to be used in phage ELISA experiments to assess binding to the composite target DNA.

Recombinants are tested directly for binding against the composite, final DNA target sequence by phage ELISA (20). Alternatively, up to two further rounds of phage selection are carried out using the composite DNA target site as bait before assaying the selected DNA-binding domains.

It should be noted that if a target DNA site contains a significant number of bases which are identical to the corresponding binding sites for the “wild type” finger on which the library is based (in this case, Zif268), it may be simpler to mutagenise the wild type finger itself (i.e., wild type Zif268). Thus, for example, one of the target sites (for Clone HIV-A′, also denoted Clone HIV-H, see Table 1 below) is amenable to this approach, since the Clone HIV-A′ site contains 8 bases which are identical to the Zif268 binding site. Clone HIV-A′ is therefore constructed by mutagenic PCR of wild-type Zif268, followed by cloning into phage and selection of the resulting clones.

The following mutagenic protocol is used. The gene coding for the three zinc fingers of the wild-type Zif268 DNA-binding domain is altered by mutagenic PCR with the following primers:

SfiVal3 (introduces a valine at position +3 of F1)
5′GCAACTGCGGCCCAGCCGGCCATGGCAGAGGAACGCCCATATGCTTGC
CCTGTCGAGTCCTGCGATCGCCGCTTTTCTCGCTCGGATGTCCTTACCC
G-3′
F1 Val +3
NotGCC (introduces mutations in f3 to allow it to bind “GCC”)
5′GAGTCATTCTGCGGCCGCGTCCTTCTGTCTTAAATGGATTTTGGTATG
CCTCTTGCGCDMGCTGKRGTSGGCAAACTTCCTCCC-3′

This generates the following Finger 3 variants:

| −1 | 1 | 2 | 3 |
|---|---|---|---|
| D | H | S | E |
| H | P | | S |
| | S | | V |
| | Y | | A |
| | | | L |

After cloning the above PCR cassette into phage vector (by standard methods, as described previously) three rounds of selection are carried out (under standard selection conditions described herein) against a DNA target site containing the sequence: 5′-GCC TGG GCG G-3′. The resulting Clone HIV-A′ (as shown in Table 1) binds its target sequence with a Kd of ˜5 nM, as measured by phage ELISA.

### Example 3

### Sequences and Properties of Isolated Three Finger Constructs

Using the above protocol, eight DNA-binding domains are produced (Table 1, Clones HIV-A to HIV-G and HIV-A′ (also known as Clone HIV-H; binds 5′-GCC TGG G(T/C)G-3′).

TABLE 1

Selection of DNA-binding domains to recognise the HIV-1 pro-
moter. Table 1 Legend:

| | |
|---|---|
| DNA target | Zinc finger |
| sequence (a) | sequence (b) |

| | | | | | | | |
|---|---|---|---|---|---|---|---|
| Clone | F1 | F2 | F3 | F1 | F2 | F3 | Kd/nM (c) |

| | | | | | | | | | | |
|---|---|---|---|---|---|---|---|---|---|---|
| | 3′-H | IJK LMN | QPQ | -5′ | −1123456 | −1123456 | −1123456 | | | |
| HIV-A | T | GCG | GAG | GGA | | RSDELTR | RSDNLST | RRDHRTT | 1.2 ± | 0.2 |
| HIV-A′ | G | GCG | GGT | CCG | | RSDVLTR | RSDHLTT | DYSVRKR | 4.9 ± | 0.4 |
| HIV-B | G | AGG | GGT | CAG | | DSAHLTR | RSDHLST | DSANRTK | 1.0 ± | 0.1 |
| HIV-C | T | ACG | TCG | TAG | | ASADLTR | NRSDLSR | TSSNRKK | 13.7 ± | 3.6 |
| HIV-D | T | TCG | TCG | ACG | | HSSDLTR | QSSDLSK | QNATRKR | 4.0 ± | 0.6 |
| HIV-E | T | CCG | AGT | CAT | | DSSSLTK | QSAHLST | DSSSRTK | 36.6 ± | 15.0 |
| HIV-F | T | CTC | TCG | AGG | | ASDDLTQ | RSSDLSR | QSAHRTK | 13.3 ± | 4.8 |
| HIV-G | G | GAT | CAA | TCG | | RSDALIQ | DRANLST | ASSTRTK | 40.3 ± | 14.6 |

(a) Nucleotide sequences from the HIV-1 promoter of the form 3′-HIJKLMNOPQ-5′, as recognised by phage clones HIV-A to HIV-G. Bases which are predicted to be bound by fingers 1 to 3 in each construct are shown. Note that the binding site for Clone HIV-A contains 5 bases from the binding site of Zif268. As a result, this clone is derived directly from Lib23, without the need for recombination. The Clone HIV-A′ site contains 8 bases which are identical to the Zif268 binding site, and is constructed by mutagenic PCR of wild-type Zif268, as described above.

(b) Amino acid sequences of the randomised helical regions of recombinant zinc finger DNA-binding domains that recognise HIV-1 sequences. Residues are numbered relative to the first helical position in each finger. Clone HIV-A, which is derived entirely from Lib23, contains some wild-type Zif268 residues. Clone HIV-A′, which is derived from Zif268 by mutagenic PCR and phage selection, is shown with wild-type residues and variant residues.

(c) Apparent Kd for the interaction of the customised DNA-binding domains for their cognate sequences as measured by phage ELISA.

Six clones (clones HIV-B to HIV-G) are engineered according to the full ‘bipartite’ protocol, while one protein (clone HIV-A) is derived directly by selection from Lib23. This illustrates a further use of the master libraries, namely to select zinc finger domains that bind DNA sequences containing the motif 5′-GCGG-3′ or 5′-GGCG-3′.

The zinc finger proteins selected for high affinity binding interact with the HIV1 promoter over a region of 130 bases, −79 to +52, where +1 is the transcription start site (see FIG. 4). Four proteins have binding sites that are dispersed upstream of the transcription initiation site (clones HIV-A to HIV-D), including two that flank the TATA box (clones HIV-C to HIV-D). Another three proteins bind to a cluster of sites at the beginning of the ORF, within the coding region for TAR (clones HIV-E to HIV-G).

HIV-A binds in the region −79 to −71 which overlaps an SPI binding site (−78 to −68). HIV-B binds the region −58 to −50 which overlaps two SP1 sites (−66 to −56 and −55 to 45). HIV-C binds the region −36 to −28 and HIV-D binds the region −22 to −14. HIV-E binds the region +22 to +30, HIV-F binds the region +33 to +41 and HIV-G binds the region +44 to +52. Clone HIV-H (HIV-A′) binds between the sites for HIV-A and HIV-B, i.e., the region −68 to −60 which overlaps two SPI binding sites (−78 to −68 and −66 to −56).

The sequence of HIV-A is
MAERPYACPVESCDRRFSRSDELTRHIRIHTGQKPFQCRICMRNFSRSDN
LSTHIRTHTGEKPFACDICGRKFARRDHRTTHTKIHLRQKD
The sequence of HIV-A′ is
MAERPYACPVESCDRRFSRSDVLTRHIRIHTGQKPFQCRICMRNFSRSDH
LTTHIRTHTGEKPFACDICGRKFADYSVRKRHTKIHLRQKD
The sequence of HIV-B is
MAERPYACPVESCDRRFSDSAHLTRHIRIHTGQKPFQCRICMRNFSRSDH
LSTHIRTHTGEKPFACDICGRKFADSANRTKHTKIHLRQKD

As the randomisations in the master libraries are restricted to amino acids with validated roles in DNA recognition, many of the recombinant DNA-binding domains make use of contacts that are consistent with the zinc finger-DNA ‘recognition code’ (21): e.g. the well-known RXD motif found at the N-terminus of many zinc finger α-helices is selected in clones A, B and G.

The different proteins bind tightly and specifically to the DNA sequences against which they are raised (Table 1, FIG. 3).

In summary, using our selection method we produce seven DNA-binding domains binding different loci in the genome of HIV-1 between positions −80 and +60 (Table 1).

### Example 4

### Production of Molecules Having High Affinity for the HIV-1 Promoter (Six Finger Constructs)

As discussed above, the invention also relates to molecules comprising multiple zinc finger motifs. One advantage of making such multifinger molecules is that they bind with greater affinity or specificity, or both, to nucleic acid target sites.

The various HIV clones binding the region of the SP1 binding sites are fused using peptide linkers in order to make six zinc finger proteins. The linker peptides are inserted between the final histidine of the first HIV clone and the first tyrosine of the second HIV clone.

HIV clones A′ and A are fused using the peptide linker sequence TGGSGGSGERP to form HIV-A′A. Clone HIV-A′A has the following amino acid sequence

MAERPYACPVESCDRRFSRSDVLTRHIRIHTGQKPFQCRICMRNFSRSDH
LTTHIRTHTGEKPFACDICGRKFADYSVRKRHTKIHTGGSGGSGERPYAC
PVESCDRRFSRSDELTRHIRIHTGQKPFQCRICMRNFSRSDNLSTHIRTH
TGEKPFACDICGRKFARRDHRTTHTKIHLRQKD

HIV clones B and A are joined using the peptide linker sequence LRQKDGGSGGSGGSGGSGGSGGSERP to form HIV-BA. Clone HIV-BA has the following amino acid sequence:

MERPYACPVESCDRRFSDSAHLTRHIRIHTGQKPFQCRICMRNFSRSDHL
STHIRTHTGEKPFACDICGRKFADSANRTKHTKIHLRQKDGGSGGSGGSG
GSGGSGGSERPYACPVESCDRRFSRSDELTRHIRIHTGQKPFQCRICMRN
FSRSDNLSTHIRTHTGEKPFACDICGRKFARRDHRTTHTKIHLRQKD

HIV clones B and A′ are fused using the peptide linker sequence TGGSGERP to form HIV-BA′. Clone HIV-BA′ has the following amino acid sequence

MAERPYACPVESCDRRFSDSAHLTRHIRIHTGQKPFQCRICMRNFSRSDH
LSTHIRTHTGEKPFACDICGRKFADSANRTKHTKIHTGGSGERPYACPVE
SCDRRFSRSDVLTRHIRIHTGQKPFQCRICMRNFSRSDHLTTHIRTHTGE
KPFACDICGRKFADYSVRKRHTKIHLRQKD

The composite fingers bind the HIV-1 target sequences with high affinity as summarised in Table 1 (also see FIG. 3).

### Example 5

### Engineering of Zinc Fingers Containing Repressor Domains

The zinc finger proteins selected to bind to the various regions of the HIV-1 promoter are engineered into repressors. These repressors contain the zinc finger DNA binding domain at the N-terminus fused in frame to the translation initiation sequence ATG. The 7 amino acid nuclear localisation sequence (NLS) of the wild-type Simian Virus 40 large-T antigen (Kalderon et al., Cell 39:499-509 (1984)) is fused to the C-terminus of the zinc finger sequence and the Kruppel-associated box (KRAB) repressor domain from human KOX1 protein (Margolin et al., PNAS 91:45094513 (1994)) is fused downstream of the NLS.

The KOX1 domain contains amino acids 1-97 from the human KOX1 protein (database accession code P21506) in addition to 23 amino acids which act as a linker. In addition, a 10 amino acid sequence from the c-myc protein (Evan et al., Mol. Cell. Biol. 5: 3610 (1985)) is introduced downstream of the KOX1 domain as a tag to facilitate expression studies of the fusion protein. The sequence of SV40-NLS-KOX1-c-myc repressor domain (NLS-KOX1-c-myc domain sequence) follows:

AARNSGPKKKRKVDGGGALSPQHSAVTQGSIIKNKEGMDAKSLTAWSRTL
VTFKDVFVDFTREEWKLLDTAQQIVYRNVMLENYKNLVSLGYQLTKPDVI
LRLEKGEEPWLVEREIHQETHPDSETAFEIKSSVEQKLISEEDL

Repressor containing polypeptides were derived from three finger constructs as well as six finger constructs (HIV-A′A-KOX, HIV-BA-KOX and HIV-BA′-KOX). Six finger proteins are created by joining the DNA binding domains of two three finger proteins together with peptide linkers. Each six finger protein contains a single KOX repressor domain.

The nucleic acid sequence of HIV A-KOX is as follows:

ATGGCAGAGCGGCCGTATGCTTGCCCTGTCGAGTCCTGCGATCGCCGCTT
TTCTCGCTCGGATGAGCTTACCCGCCATATCCGCATCCACACAGGCCAGA
AGCCCTTCCAGTGTCGAATCTGCATGCGTAACTTCAGTCGTAGTGACAAC
CTGAGCACGCACATCCGCACCCACACAGGCGAGAAGCCTTTTGCCTGTGA
CATTTGTGGGAGGAAATTTGCCCGGAGGGACCACCGCACAACGCATACCA
AGATACACCTGCGCCAAAAAGATGCGGCCCGGAATTCCGGCCCAAAAAAG
AAGAGAAAGGTCGACGGCGGTGGTGCTTTGTCTCCTCAGCACTCTGCTGT
CACTCAAGGAAGTATCATCAAGAACAAGGAGGGCATGGATGCTAAGTCAC
TAACTGCCTGGTCCCGGACACTGGTGACCTTCAAGGATGTATTTGTGGAC
TTCACCAGGGAGGAGTGGAAGCTGCTGGACACTGCTCAGCAGATCGTGTA
CAGAAATGTGATGCTGGAGAACTATAAGAACCTGGTTTCCTTGGGTTATC
AGCTTACTAAGCCAGATGTGATCCTCCGGTTGGAGAAGGGAGAAGAGCCC
TGGCTGGTGGAGAGAGAAATTCACCAAGAGACCCATCCTGATTCAGAGAC
TGCATTTGAAATCAAATCATCAGTTGAACAAAAACTTATTTCTGAAGAAG
ATCTGTAA

The amino acid sequence of HIV A-KOX is as follows:

MAERPYACPVESCDRRFSRSDELTRHIRIHTGQKPFQCRICMRNFSRSDN
LSTHIRTHTGEKPFACDICGRKFARRDHRTTHTKIHLRQKDAARNSGPKK
KRKVDGGGALSPQHSAVTQGSIIKNKEGMDAKSLTAWSRTLVTFKDVFVD
FTREEWKLLDTAQQIVYRNVMLENYKNLVSLGYQLTKPDVILRLEKGEEP
WLVEREIHQETHPDSETAFEIKSSVEQKLISEEDL.

The nucleic acid sequence of HIV A′-KOX is as follows:

ATGGCAGAACGCCCGTATGCTTGCCCTGTCGAGTCCTGCGATCGCCGCTT
TTCTCGCTCGGATGTCCTTACCCGCCATATCCGCATCCACACAGGCCAGA
AGCCCTTCCAGTGTCGAATCTGCATGCGTAACTTCAGTCGTAGTGACCAC
CTTACCACCCACATCCGCACCCACACAGGCGAGAAGCCTTTTGCCTGTGA
CATTTGTGGGAGGAAGTTTGCCGACTACAGCGTACGCAAGAGGCATACCA
AAATCCATCTGCGCCAAAAAGATGCGGCCCGGAATTCCGGCCCAAAAAAG
AAGAGAAAGGTCGACGGCGGTGGTGCTTTGTCTCCTCAGCACTCTGCTGT
CACTCAAGGAAGTATCATCAAGAACAAGGAGGGCATGGATGCTAAGTCAC
TAACTGCCTGGTCCCGGACACTGGTGACCTTCAAGGATGTATTTGTGGAC
TTCACCAGGGAGGAGTGGAAGCTGCTGGACACTGCTCAGCAGATCGTGTA
CAGAAATGTGATGCTGGAGAACTATAAGAACCTGGTTTCCTTGGGTTATC
AGCTTACTAAGCCAGATGTGATCCTCCGGTTGGAGAAGGGAGAAGAGCCC
TGGCTGGTGGAGAGAGAAATTCACCAAGAGACCCATCCTGATTCAGAGAC
TGCATTTGAAATCAAATCATCAGTTGAACAAAAACTTATTTCTGAAGAAG
ATCTGTAA

The amino acid sequence of HIV A′-KOX is as follows:

MERPYACPVESCDRRFSRSDVLTRHIRIHTGQKPFQCRICMRNFSRSDHL
TTHIRTHTGEKPFACDICGRKFADYSVRKRHTKIHLRQKDAARNSGPKKK
RKVDGGGALSPQHSAVTQGSIIKNKEGMDAKSLTAWSRTLVTFKDVFVDF
TREEWKLLDTAQQIVYRNVMLENYKNLVSLGYQLTKPDVILRLEKGEEPW
LVEREIHQETHPDSETAFEIKSSVEQKLISEEDL.

The nucleic acid sequence of HIVB-KOX is as follows:

ATGGCGGAGAGGCCCTACGCATGCCCTGTCGAGTCCTGCGATCGCCGCTT
TTCTGACTCGGCCCACCTTACCCGGCATATCCGCATCCACACCGGTCAGA
AGCCCTTCCAGTGTCGAATCTGCATGCGTAACTTCAGTCGGAGCGACCAC
CTGAGCACCCACATCCGCACCCACACAGGCGAGAAGCCTTTTGCCTGTGA
CATTTGTGGGAGGAAATTTGCCGACAGCGCCAACCGCACAAAGCATACCA
AGATACACCTGCGCCAAAAAGATGCGGCCCGGAATTCCGGCCCAAAAAAG
AAGAGAAAGGTCGACGGCGGTGGTGCTTTGTCTCCTCAGCACTCTGCTGT
CACTCAAGGAAGTATCATCAAGAACAAGGAGGGCATGGATGCTAAGTCAC
TAACTGCCTGGTCCCGGACACTGGTGACCTTCAAGGATGTATTTGTGGAC
TTCACCAGGGAGGAGTGGAAGCTGCTGGACACTGCTCAGCAGATCGTGTA
CAGAAATGTGATGCTGGAGAACTATAAGAACCTGGTTTCCTTGGGTTATC
AGCTTACTAAGCCAGATGTGATCCTCCGGTTGGAGAAGGGAGAAGAGCCC
TGGCTGGTGGAGAGAGAAATTCACCAAGAGACCCATCCTGATTCAGAGAC
TGCATTTGAAATCAAATCATCAGTTGAACAAAAACTTATTTCTGAAGAAG
ATCTGTAA

The amino acid sequence of HIVB-KOX is as follows:

MAERPYACPVESCDRRFSDSAHLTRHIRIHTGQKPFQCRICMRNFSRSDH
LSTHIRTHTGEKPFACDICGRKFADSANRTKHTKIHLRQKDAARNSGPKK
KRKVDGGGALSPQHSAVTQGSIIKNKEGMDAKSLTAWSRTLVTFKDVFVD
FTREEWKLLDTAQQIVYRNVMLENYKNLVSLGYQLTKPDVILRLEKGEEP
WLVEREIHQETHPDSETAFEIKSSVEQKLISEEDL.

The nucleic acid sequence of HIV A′A-KOX is as follows:

ATGGCAGAACGCCCGTATGCTTGCCCTGTCGAGTCCTGCGATCGCCGCTT
TTCTCGCTCGGATGTCCTTACCCGCCATATCCGCATCCACACAGGCCAGA
AGCCCTTCCAGTGTCGAATCTGCATGCGTAACTTCAGTCGTAGTGACCAC
CTTACCACCCACATCCGCACCCACACAGGCGAGAAGCCTTTTGCCTGTGA
CATTTGTGGGAGGAAGTTTGCCGACTACAGCGTACGCAAGAGGCATACCA
AAATCCATACCGGCGGGAGCGGCGGGAGCGGCGAGCGGCCGTATGCTTGC
CCTGTCGAGTCCTGCGATCGCCGCTTTTCTCGCTCGGATGAGCTTACCCG
CCATATCCGCATCCACACAGGCCAGAAGCCCTTCCAGTGTCGAATCTGCA
TGCGTAACTTCAGTCGTAGTGACAACCTGAGCACGCACATCCGCACCCAC
ACAGGCGAGAAGCCTTTTGCCTGTGACATTTGTGGGAGGAAATTTGCCCG
GAGGGACCACCGCACAACGCATACCAAGATACACCTGCGCCAAAAAGATG
CGGCCCGGAATTCCGGCCCAAAAAAGAAGAGAAAGGTCGACGGCGGTGGT
GCTTTGTCTCCTCAGCACTCTGCTGTCACTCAAGGAAGTATCATCAAGAA
CAAGGAGGGCATGGATGCTAAGTCACTAACTGCCTGGTCCCGGACACTGG
TGACCTTCAAGGATGTATTTGTGGACTTCACCAGGGAGGAGTGGAAGCTG
CTGGACACTGCTCAGCAGATCGTGTACAGAAATGTGATGCTGGAGAACTA
TAAGAACCTGGTTTCCTTGGGTTATCAGCTTACTAAGCCAGATGTGATCC
TCCGGTTGGAGAAGGGAGAAGAGCCCTGGCTGGTGGAGAGAGAAATTCAC
CAAGAGACCCATCCTGATTCAGAGACTGCATTTGAAATCAAATCATCAGT
TGAACAAAAACTTATTTCTGAAGAAGATCTGTAA

The amino acid sequence of HIV A′A-KOX is as follows:

MAERPYACPVESCDRRFSRSDVLTRHIRIHTGQKPFQCRICMRNFSRSDH
LTTHIRTHTGEKPFACDICGRKFADYSVRKRHTKIHTGGSGGSGERPYAC
PVESCDRRFSRSDELTRHIRIHTGQKPFQCRICMRNFSRSDNLSTHIRTH
TGEKPFACDICGRKFARRDHRTTHTKIHLRQKDAARNSGPKKKRKVDGGG
ALSPQHSAVTQGSIIKNKEGMDAKSLTAWSRTLVTFKDVFVDFTREEWKL
LDTAQQIVYRNVMLENYKNLVSLGYQLTKPDVILRLEKGEEPWLVEREIH
QETHPDSETAFEIKSSVEQKLISEEDL . . .

The nucleic acid sequence of HIVBA-KOX is as follows:

ATGGCGGAGAGGCCCTACGCATGCCCTGTCGAGTCCTGCGATCGCCGCTT
TTCTGACTCGGCCCACCTTACCCGGCATATCCGCATCCACACCGGTCAGA
AGCCCTTCCAGTGTCGAATCTGCATGCGTAACTTCAGTCGGAGCGACCAC
CTGAGCACCCACATCCGCACCCACACAGGCGAGAAGCCTTTTGCCTGTGA
CATTTGTGGGAGGAAATTTGCCGACAGCGCCAACCGCACAAAGCATACCA
AGATACACCTGCGCCAAAAAGATGGGGGCAGCGGCGGGTCCGGGGGGAGC
GGCGGCTCCGGGGGCAGCGGCGGGTCCGAGCGGCCGTATGCTTGCCCTGT
CGAGTCCTGCGATCGCCGCTTTTCTCGCTCGGATGAGCTTACCCGCCATA
TCCGCATCCACACAGGCCAGAAGCCCTTCCAGTGTCGAATCTGCATGCGT
AACTTCAGTCGTAGTGACAACCTGAGCACGCACATCCGCACCCACACAGG
CGAGAAGCCTTTTGCCTGTGACATTTGTGGGAGGAAATTTGCCCGGAGGG
ACCACCGCACAACGCATACCAAGATACACCTGCGCCAAAAAGATGCGGCC
CGGAATTCCGGCCCAAAAAAGAAGAGAAAGGTCGACGGCGGTGGTGCTTT
GTCTCCTCAGCACTCTGCTGTCACTCAAGGAAGTATCATCAAGAACAAGG
AGGGCATGGATGCTAAGTCACTAACTGCCTGGTCCCGGACACTGGTGACC
TTCAAGGATGTATTTGTGGACTTCACCAGGGAGGAGTGGAAGCTGCTGGA
CACTGCTCAGCAGATCGTGTACAGAAATGTGATGCTGGAGAACTATAAGA
ACCTGGTTTCCTTGGGTTATCAGCTTACTAAGCCAGATGTGATCCTCCGG
TTGGAGAAGGGAGAAGAGCCCTGGCTGGTGGAGAGAGAAATTCACCAAGA
GACCCATCCTGATTCAGAGACTGCATTTGAAATCAAATCATCAGTTGAAC
AAAAACTTATTTCTGAAGAAGATCTGTAA

The amino acid sequence of HIVBA-KOX is as follows:

MAERPYACPVESCDRRFSDSAHLTRHIRIHTGQKPFQCRICMRNFSRSDH
LSTHIRTHTGEKPFACDICGRKFADSANRTKHTKIHLRQKDGGSGGSGGS
GGSGGSGGSERPYACPVESCDRRESRSDELTRHIRIHTGQKPFQCRICMR
NFSRSDNLSTHIRTHTGEKPFACDICGRKFARRDHRTTHTKIHLRQKDAA
RNSGPKKKRKVDGGGALSPQHSAVTQGSIIKNKEGMDAKSLTAWSRTLVT
FKDVFVDFTREEWKLLDTAQQIVYRNVMLENYKNLVSLGYQLTKPDVILR
LEKGEEPWLVEREIHQETHPDSETAFEIKSSVEQKLISEEDL.

The nucleic acid sequence of HIVBA′-KOX is as follows:

ATGGCGGAGAGGCCCTACGCATGCCCTGTCGAGTCCTGCGATCGCCGCTT
TTCTGACTCGGCCCACCTTACCCGGCATATCCGCATCCACACCGGTCAGA
AGCCCTTCCAGTGTCGAATCTGCATGCGTAACTTCAGTCGGAGCGACCAC
CTGAGCACCCACATCCGCACCCACACAGGCGAGAAGCCTTTTGCCTGTGA
CATTTGTGGGAGGAAATTTGCCGACAGCGCCAACCGCACAAAGCATACCA
AGATACACACCGGCGGGAGCGGCGAGCGGCCGTATGCTTGCCCTGTCGAG
TCCTGCGATCGCCGCTTTTCTCGCTCGGATGTCCTTACCCGCCATATCCG
CATCCACACAGGCCAGAAGCCCTTCCAGTGTCGAATCTGCATGCGTAACT
TCAGTCGTAGTGACCACCTTACCACCCACATCCGCACCCACACAGGCGAG
AAGCCTTTTGCCTGTGACATTTGTGGGAGGAAGTTTGCCGACTACAGCGT
GCGCAAGAGGCATACCAAAATCCATTTAAGACAGAAGGACGCGGCCCGGA
ATTCCGGCCCAAAAAAGAAGAGAAAGGTCGACGGCGGTGGTGCTTTGTCT
CCTCAGCACTCTGCTGTCACTCAAGGAAGTATCATCAAGAACAAGGAGGG
CATGGATGCTAAGTCACTAACTGCCTGGTCCCGGACACTGGTGACCTTCA
AGGATGTATTTGTGGACTTCACCAGGGAGGAGTGGAAGCTGCTGGACACT
GCTCAGCAGATCGTGTACAGAAATGTGATGCTGGAGAACTATAAGAACCT
GGTTTCCTTGGGTTATCAGCTTACTAAGCCAGATGTGATCCTCCGGTTGG
AGAAGGGAGAAGAGCCCTGGCTGGTGGAGAGAGAAATTCACCAAGAGACC
CATCCTGATTCAGAGACTGCATTTGAAATCAAATCATCAGTTGAACAAAA
ACTTATTTCTGAAGAAGATCTGTAA

The amino acid sequence of HIVBA′-KOX is as follows:

MAERPYACPVESCDRRFSDSAHLTRHIRIHTGQKPFQCRICMRNFSRSDH
LSTHIRTHTGEKPFACDICGRKFADSANRTKHTKIHTGGSGERPYACPVE
SCDRRFSRSDVLTRHIRIHTGQKPFQCRICMRNFSRSDHLTTHIRTHTGE
KPFACDICGRKFADYSVRKRHTKIHLRQKDAARNSGPKKKRKVDGGGALS
PQHSAVTQGSIIKNKEGMDAKSLTAWSRTLVTFKDVFVDFTREEWKLLDT
AQQIVYRNVMLENYKNLVSLGYQLTKPDVILRLEKGEEPWLVEREIHQET
HPDSETAFEIKSSVEQKLISEEDL.

### Example 6

### Modulation of Transcription in a Model System (CAT Assay)

Modulation of transcription of nucleic acid molecules according to the invention is assayed using transient HIV1 promoter reporter assays. The zinc fingers selected for high affinity binding to the HIV-1 promoter in the preceding Examples are tested for activity using a CAT reporter vector containing the HIV-1 promoter placed upstream of a chloramphenicol acetyl transferase coding region.

COS7 cells are used for transient assays and are grown according to the suppliers instructions in DMEM media supplemented with penicillin/streptomycin, L-glutamine and foetal calf serum. Cells are split 1:3 the day prior to transfection. Cells are washed and resuspended in PBS at a concentration of 1×10⁷ cells/ml.

0.7 ml of cells are transfected with transfection mix by electroporation in a 0.4 cm gap electroporation cuvette at 1.9 kV and 25 μF. In this Example, the transfection mix-comprises 10 μg HIV-1 promoter reporter plasmid, 0.1 μg Tat expressing plasmid and 10 μg HIV zinc finger expressing plasmid. For control transfections, the Tat expressing plasmid and the HIV zinc finger expressing plasmid, or just the HIV zinc finger expressing plasmid, are substituted by a plasmid expressing lacZ from the same CMV promoter.

The electroporated samples are transferred to 100 mm diameter cell culture plates containing 8 ml Cos7 growth media and incubated for 24 hours at 37° C. and 5% CO₂.

Cells are harvested using trypsin/EDTA into 5 mls PBS and pefleted at 1000 rpm for 5 minutes at room temperature. Pellets are resuspended in 1 ml PBS, 200 μl is removed for normalisation of total protein content using the Biorad protein Assay (Biorad). The remaining cells are pelleted as described previously, pellets are resuspended in 800 μl 1× reporter lysis buffer (Promega). Samples are spun at 12000 rpm for 2 minutes at room temperature. 400 μl supernatant is analysed for CAT activity using the Quan-T-CAT assay system (Amersham Pharmacia Life Sciences) according to the manufacturer's instructions with a 10 minute 37° C. incubation.

The streptavidin coated polystyrene beads pelleted at the end of the CAT assay are resuspended in 1 ml liquid scintillation cocktail (Beckman) and counted for the presence of ³H for 5 minutes in a scintillation counter. Counts per minute are normalised for transfection efficiency and cell number prior to analysis.

Results from the transient reporter assays are summarised in FIG. 5. Background expression from the HIV 1 promoter is activated 14 fold by the action of the HIV Tat protein. A series of 3 zinc finger proteins containing repressors (HIV-A to HIV-F) and six zinc finger proteins (HIV-A′A, HIV-BA and HIV-BA′) are tested as fusions with the KOX repressor domain for their ability to repress the activated promoter.

The three finger proteins are shown to repress transcription of the HIV-1 promoter. Expression of the three finger protein HIV-B-KOX significantly represses the HIV promoter 7 fold from its Tat-activated level.

Zinc finger repressor proteins are also tested in combination with each other. Such combinations are HIV-A-KOX protein with HIV-A′-KOX, HIV-A-KOX with HIV-B-KOX and HIV-A′-KOX with HIV-B-KOX. Each of the combinations repress the activated HIV promoter to a greater extent than the single HIV-B-KOX three finger protein alone. These combinations repress the HIV-1 promoter 11 fold, 12 fold and 10 fold respectively (FIG. 5).

Six finger constructs containing repressors are assayed against the activated HIV-1 promoter. These six finger proteins repress the expression of CAT to different levels with HIV-BA-KOX and HIV-BA′-KOX being the most active. Both these two six finger proteins significantly repress the activated promoter to levels below background expression of the HIV promoter. The magnitude of the repression from the activated level is 21 fold for HIV-BA-KOX and 48 fold for HIV-BA′-KOX (FIG. 5).

These data demonstrate the significant advantages and utility of engineering zinc finger proteins that target endogenous transcription factor binding sites. It is particularly useful to target multiple endogenous transcription factor binding sites and the present invention demonstrates this using combinations of zinc finger proteins (e.g. HIV-A-KOX+HIV-A′-KOX; HUV-A-KOX+HIV-B-KOX; HIV-A′-KOX+HIV-B-KOX) and using single zinc finger proteins which are engineered to target sequences which span endogenous transcription factor binding sites (e.g. HIV-BA-KOX, HIV-BA′-KOX and HIV-A′A-KOX).

### Example 7

### Modulation of Enhanced Transcription of Nucleic Acid Molecules in a Physiological Cellular System (Luciferase Assay)

The purpose of this experiment is to assay inhibition of HIV1 promoter by zinc finger repressors in the context of a T cell, which is the natural host of HIV1. The Jurkat T cell line is used. This line overexpresses the endogenous transcription factor NF-κB, which is a potent activator of the HIV LTR, in response to stimulation by PMA (Phorbol-myristyl-acetate) and PHA (Phytohaemagluttinin). The zinc fingers are tested under these conditions. In addition, a different reporter system, luciferase, is used, showing that inhibition of transcription is dependent on the HIV promoter, rather than the reporter gene.

Plasmids

The luciferase reporter plasmid containing the wild-type HIV-1 LTR (LTR-FF) is generated by cloning the Eco RV to Hind III fragment of D5-3-3 (Dingwall et al, 1990) into the Sma I and Hind III sites of pGL3 basic (Promega).

Transfection of Cells

The Jurkat human T-cell line is cultured at 37° C. in 7% CO₂ in RPMI 1640 media containing penicillin (100U/ml) and streptomycin (100 μg/ml) supplemented with 10% FCS.

Transfections are carried out in 6-well plates using 600 ng of LTR-FF, 0-50 ng of C63-4-1, which expresses Tat in trans from a Molony virus LTR (Dingwall et al, 1989), and 150 ng of pRL-TK (Pr.omega). pRL-TK contains the Renilla luciferase gene under the control of the TK promoter and-is used as an internal control for transfection efficiency. PUC12 DNA is used to keep the amounts of plasmid DNA constant in samples containing no C63-4-1. Samples also contained 150 ng of control vector DNA (pcDNA 3.1(−)), or 150 ng of the zinc finger-expressing plasmids TFIIIAZif-KOX, BA′-KOX or BA′. DNA is mixed in a total volume of 150 μl of EC buffer (Qiagen) and 8 μl of Enhancer added for every μg of DNA present. Samples are then vortexed and incubated at RT for 5 mins prior to the addition of Effectene (10 μl for every μg of DNA). Samples are incubated for a further 5 minutes at RT and 0.5 ml of normal growth media then added. The total mix is then added to 2 mls of cells resuspended at 2.5×10⁵/ml in fresh media. The cells are incubated at 37° C. for 2 hrs and 2.5 mls of normal growth media is then added.

Cells are activated 24 hrs after transfection by the addition of Phytohaemagluttinin (PHA) (SIGMA) to a final concentration of 10 μg/ml and Phorbol-myristyl-acetate (PMA) (SIGMA) to a final concentration of 50 ng/ml.

Luciferase Assays

Cells are harvested 48 hrs after transfection, washed once in PBS and then lysed in 150 μl of 1×PLB (Passive lysis buffer, Promega) for 30 mins at RT. Lysates (10 μl) are assayed using 50 μl of LAR II reagent and 50 μl of Stop and Glo reagent from the Dual luciferase assay system kit (Promega). Firefly luciferase and Renilla luciferase activity is measured sequentially using a microplate luminometer with an injection unit (Berthold detection systems). Firefly luminescence is measured for a period of 1 second after a delay of 2 seconds following the addition of LAR II and Renilla luminescence is measured for 1 second following a 2 second delay after the addition of Stop and Glo reagent.

Toxicity Assays

Toxicity assays are performed in parallel with luciferase assays by transferring 100 μl of transfected cell mix to a 96-well plate. 100 μl of normal growth media is then added 2 hrs post-transfection. These cells are treated in parallel with PMA and PHA on day 2 and cell proliferation is measured on day 3 by the addition of 40 μl of CellTiter 96 Aqueous one solution cell proliferation assay reagent (Promega). Cells are then incubated at 37° C. for 24 hrs and the level of coloured product produced is determined by measuring the absorbance at 490 nm.

Results

A. Determination of the Optimal Concentrations of PMA and Tat

Initial experiments are performed to determine the optimal amount of Phorbol myristyl acetate required to stimulate the maximal level of basal HIV transcription and the optimal concentration of Tat required for full activation of the LTR. Jurkat T-cells are transfected with a reporter construct containing the HIV LTR upstream of the firefly luciferase gene. Increasing concentrations of the Tat-expressing plasmid C63-4-1 are included in the transfections and cells are treated with a combination of PHA and PMA 24 hrs post-transfection. PHA is used at a final concentration of 10 μg/ml and the concentration of PMA is titrated from 25 ng/ml to 50 ng/ml. We observe a maximal Tat transactivation using 25 ng of C63-4-1 (FIG. 6A). Concentrations of C634-1 between 20 and 50 ng/ml are tested in later experiments (see below). Consistent with our previous results, the concentration of PMA required to give the maximal level of transcriptional activation is 50 ng/ml. Concentrations of PMA higher than 50 ng/ml are not tested since toxicity effects are apparent even at 50 ng/ml (see below).

B. pHIV-BA′-KOX Inhibits HIV Transcription in T-Cells

Experiments are performed to determine whether the expression of LTR-binding zinc finger proteins can inhibit HIV transcription in T-cells. For these initial experiments we use the plasmid pHIVBA′-KOX which expresses the 6-finger protein BA′ as a fusion with the transcriptional repression domain of the KOX protein. We examine the effect of expressing BA′-KOX in trans on transcription in the absence and presence of Tat, and in the absence and presence of PMA and PHA. The amount of C63-4-1 included in the transfections is titrated further and 40 ng is found to give the best Tat transactivation. This concentration of C634-1 is used in further experiments. The inclusion of 150 ng of pHIVBA′-KOX plasmid in these transfections is sufficient to inhibit transcription in the absence and presence of Tat and in the presence of PMA and PHA (FIG. 6B). In fact the level of transcription detected in activated cells in the presence of Tat is inhibited by 88% in the presence of 150 ng of pHIV BA′-KOX. Increasing the amount of the pHIV-BA′-KOX plasmid included to 300 ng does not result in significant increases in inhibition. Since BA′-KOX is able to efficiently inhibit transcription in the presence of PMA and PHA, it is clear that the binding of NF-KB to its upstream binding sites cannot overcome the inhibitory function of this molecule.

C. The Inhibitory Function of BA′-KOX is Mediated by the KOX Domain

Further experiments are performed to determine whether the binding of HIV-BA′ to the HIV LTR is able to inhibit transcription in the absence of the KOX domain. These experiments are performed using 150 ng of each of the expression plasmids pHIV-BA′ and pHIV-BA′-KOX. As an additional control for any non-specific effects resulting from the expression of the zinc finger proteins or KOX domain, we also perform transfections using 150 ng of a vector expressing the zinc finger fusion protein, TFZ-KOX, which does not bind to the HIV LTR. The pRL-TK plasmid is also included in these and all subsequent experiments as a control for transfection efficiency. This plasmid expresses the Renilla luciferase gene under the control of the HSV TK promoter. Toxicity assays are also performed in parallel to enable us to account for the toxic effects of PMA and PHA and to detect any possible toxicity effects of the zinc finger expressing plasmids. All results are corrected for toxicity and the HIV LTR firefly luciferase results are then adjusted for transfection efficiency. The expression of TFZ-KOX in these cells has no effect on HIV transcription as expected and provides an important control for any possible trans effects of the KOX repression domain (FIG. 6C). The expression of HIV-BA′-KOX inhibits HIV transcription effectively, but the expression of BA′ without the KOX domain has a stimulatory effect on transcription particularly in the presence of PMA and PHA. It is clear from this experiments that the inhibitory function of HIV-BA′-KOX is mediated by the repression domain and is not the result on any inhibition of Sp1 or polII binding to the LTR. The stimulatory effect of BA′ may result from the opening up of the DNA structure around the promoter allowing easier access for transcription factors such as NF-κB.

D. Six Finger Proteins are More Effective Inhibitors than 3 Finger Proteins

The six finger protein pHIV-BA′ contains two 3 finger domains which bind to two separate sites in the HIV LTR. We investigate whether the expression of the HIV-B or HIV-A′ three finger binding domains separately results in more effective inhibition of HIV transcription. We perform experiments to compare the extent of inhibition obtained using pHIV-BA′-KOX pHIV-B-KOX, or pHIV-A′-KOX, alone and in combination. The results shown in FIG. 7A demonstrate that the three finger domains are less effective at inhibiting HIV transcription. pHIV-B-KOX or pHIV-A′-KOX alone reduce the level of activated transcription in the presence of Tat by 55% and 17% respectively, compared to the 89% inhibition observed with pHV-BA′-KOX. The expression of both of these 3-finger proteins in combination produces more efficient inhibition, reducing the level of activated transcription in the presence of Tat by 66% of wild-type levels. The varying degrees of inhibition obtained using these constructs may result from the different binding affinities of the zinc finger proteins to their target sites.

E. pHIV-AB-KOX Inhibits HIV Transcription as Efficiently as pHIV-BA′-KOX

The HIV-A′ zinc finger binding site is located immediately downstream of the NF-kB sites in the LTR. The ability of HIV-BA′-KOX to target the KOX repression domain close to the NF-κB sites may be important for the inhibition of activated transcription by this molecule. We investigate the possibility that a fusion protein which recognizes another site close to the A′ site might also be able to inhibit transcription effectively. This peptide, HIV-AB-KOX, binds to the A site, which is located slightly upstream from the A′ site, and to the B site, which is also recognized by HIV-BA′-KOX. This zinc finger protein inhibits HIV transcription, and in particular, activates transcription to the same extent as HIV-BA′-KOX (FIG. 7B). Activated transcription in the presence of Tat is inhibited by 92% and 96% in the presence of 150 ng of pHIV-BA′-KOX or 150 ng of pHIV-AB-KOX, respectively.

### Example 8

### Transfection of DNA Constructs and Challenge With HIV-1

NP2/CD4 cells are set up at 10⁵ cells per well in 6-well trays in DMEM, 5% foetal calf serum and antibiotics. NP2 cells are a human glioma cell line that do not express the common HIV and SIV coreceptors (Soda, Y., N. Shimizu, A. Jinno, H. Y. Liu, K. Kanbe, T. Kitamura, and H. Hoshino. 1999. Establishment of a new system for determination of coreceptor usages of HIV based on the human glioma NP-2 cell line. Biochem. Biophys. Res. Commun. 258:313-321).

The following day, various combinations of plasmid DNA are transfected with and without the pcDNA3.1/CXCR4 expression construct. Transfections are carried out using lipofectin (Gibco) following the maker's instructions. 1 day after transfection, the cells are trypsinised and reseeded into 48 well trays at 2.5×10⁴ cells per well and reincubated.

The next day, the transfected cells are challenged with tenfold serial dilutions of the HXB2 strain of HIV-1. 100 μl of virus supernatant is added to the wells and incubated for 3 hours, after which 1 ml of growth medium is added and the infected cells incubated. After 3 days, the cells are washed in PBS and fixed in cold (40° C.) methanol acetone 1:1 for ten minutes. After further PBS and PBS+1% FCS washes, the cells are immunostained using p24 monoclonal antibodies, followed by an anti-mouse IgG-β-galactosidase and then enzyme substrate as described previously (Simmons, G., A. McKnight, Y. Takeuchi, H. Hoshino, and P. R. Clapham. 1995. Cell-to-cell fusion, but not virus entry in macrophages by T-cell line tropic HIV-1 strains: a V3 loop-determined restriction. Virology. 209:696-700). Foci of infection stained blue and are estimated by light microscopy.

Results of DNA Constructs and Challenge With HIV-1

The results of the live virus assays, which were performed in duplicate, demonstrate that the specific zinc finger for the HIV-1 LTR (pHIVBA′-KOX) represses HIV-1 (HXB2 strain) replication in human cell culture (Table 2 below). Repression does not occur when a control zinc finger repressor (pTFZ KOX) that is specific for a different DNA sequence is used, thus showing that repression is not attributable to non-specific repression from the KOX domain. Zinc finger alone, pHIVBA′, without a repression domain, also represses viral replication but to a lesser extent than pHIV-BA′-KOX.

TABLE 2
Total Numbers of Foci Formed from Infection with HIV-1 in Human
NP2 Cells Transfected with Co-receptor and Zinc Finger

| | HXB2 Foci of infection per well (in |
|---|---|
| | duplicate) |
| Transfected | Virus ¼ dilution |
| 1. pTFZ-KOX + CXCR4 | 72, 81 |
| 2. pHIV-BA′-KOX + CXCR4 | 10, 15 |
| 3. pHIV BA′ + CXCR4 | 40, 36 |
| 4. CXCR4 only | 53, 67 |
| 5. nothing | 0, 0 |

The data shown in this Example demonstrates that zinc fingers according to the present invention are effective in reducing infection with HIV virus.

### Example 9

### Delivery of Zinc Fingers to Human Cells Using a Viral Vector

The oncoretroviral vector used contains HIV-BA′-KOX gene and cis-acting viral sequences for gene expression and viral replication, such as the Long Terminal Repeat (LTR), the primer binding site, the attachment site and polypurine tract sequences and an extended packaging signal. It has been deleted of all viral protein coding sequences so that it is not replication competent This vector has been used in many gene therapy clinical trials and has shown no sign of toxicity either ex vivo or in patient treated.

The HIV-BA′-KOX gene extracted from the pcDNA3.1 plasmid using the PME1 restriction enzyme is cloned by standard genetic engineering methods into an LNL-type vector inserted into a pUC backbone. The expression of both HIV-BA′-KOX is placed under the transcriptional control of the Moloney murine leukemia virus (Mo-MuLV) long terminal repeat (LTR). The viral vector also encodes a marker protein, the green fluorescent protein (GFP). The expression of this marker gene is also driven by the viral LTR, a mechanism made possible by the insertion of an internal ribosomal entry site (IRES) sequence between both genes.

The helper functions essential to propagate the retroviral vector, such as replication and production of a functional viral capsid, may be provided by helper cells (packaging cell line) or by co-transfected plasmids.

Viral supernatant is produced by transient transfection of 293T cells, as described in detail in the following Example. The helper functions are provided from two different constructs, one expressing Gag-Pol encoding the viral capsid, reverse transcriptase and integrase but lacking the encapsidation signal normally present in the Gag region and another expressing the envelope. For successful infection of human cells, the envelope used derives from the feline endogenous retrovirus (RD114) envelope protein but alternatively the Gibbon Ape Leukemia virus (GALV) envelope protein or the G protein of vesicular stomatitis virus (VSV-G) may be used.

Oncoretroviral Vector Production

RD114 pseudotyped vectors are produced by transient transfection of three plasmids into 293T cells: the transfer vector plasmid (LNL-based), pHIT60 (from Prof Mary Collins' lab, UCL, London, UK) a helper packaging plasmid encoding GAG and POL proteins of murine leukemia virus, and pRDF (from Prof Mary Collins' lab, UCL, London, UK) encoding for feline endogenous retrovirus (RD114) envelope protein.

A total of 1.5×10⁷ 293T cells are seeded in one 150-cm² flask over-night prior to transfection Cells are cultured at 37° C. in Dulbecco's modified Eagle medium (DMEM) with 10% fetal calf serum (FCS) in a 5% CO₂ incubator. A total of 72 μg of plasmid DNA is used for the transfection of one flask: 12 μg of the envelope plasmid (pRDF), 24 μg of packaging plasmid (pHIT60), and 36 μg of transfer vector (pRetro) plasmid are pre-complex with lipofectamine 2000 (life technology) in Optimem according to the manufacturer instructions. The DNA plus lipofectamine complexes are then added to the cells. After 4 hours incubation at 37° C. in a 5% CO₂ incubator, the medium is replaced by fresh DMEM or alternatively RPMI supplemented with 10% FCS and further incubated at 33° C. to enhance the stability of the recombinant virus. At 36 hours and 60 hours post-transfection, the medium is harvested, cleared by low-speed centrifugation (1200 rpm, 5 min), filtered through 0.45-μm-pore-size filters and use directly or kept at −80° C.

Transduction of Human Cells

Hela and Jurkat cell are then infected with the recombinant viral vector encoding the HIV-BA′-KOX gene. An empty viral vector containing the GFP gene is used as control.

Hela cell line, a human cell line, is grown according to supplier instruction in DMEM L-glutamine containing medium supplemented with penicillin/streptavidin and fetal calf serum (complete DMEM). For successful infection with the recombinant viral vector, cells are harvested using trypsin/EDTA and 10⁵ cells are plated into a 6 well-cell culture plate containing 4 ml of viral supernatant. Cells are then further incubated for three to five days at 33° C. in 5% CO₂.

The Jurkat T cell line, a human derived lymphoblast T cell, is grown according to supplier instruction in RPMI 16100 L-glutamine containing medium supplemented with penicillin/streptavidin and fetal calf serum (complete RPMI). Cells are resuspended in 3 ml of freshly harvested retroviral supernatant and added at the concentration of 10⁵/well to a 6 well non-tissue culture treated plate (Becton Dickinson) pre-coated with 15 μg/cm2 retronectin (TaKaRa, Shiga, Japan). Plates are then incubated for 16 hours at 33° C. A total of 2 rounds of infection are performed in which two-third of the medium is replaced with viral supernatant. At the end of the transduction protocol cells are harvested using complete RPMI.

### Example 10

### Detection of HIV-BA′-KOX Protein in Transduced Cells

After three to five days post infection, the successful delivery of the HIV-BA′-KOX construct into Hela and Jurkat T-cells is assayed by immunochemistry (FIG. 17).

HeLa cells, used as control, are transfected by electroporation with 20 μg pcmv-HIV-BA′-KOX. These cells are seeded along with viral infected HeLa cells expressing HIV-BA′-KOX, control viral infected HeLa cells not expressing HIV-BA′-KOX and Uninfected HeLa cells, at 2.5×10⁵ cells per well into 2 wells each of an 8-well chamber slide (Life Technologies). The cells are incubated at 37° C., 5% CO₂ for 16 hrs.

Media is removed from each well and the cells washed twice per well with phosphate buffered saline (PBS). Samples are fixed for 20 minutes at 4° C. in 4% paraformaldehyde in PBS then washed twice with PBS. Samples are permeablised for 10 minutes at 22° C. in 0.25% triton-X100 in PBS and washed twice with PBS. Samples are blocked for 15 minutes at 22° C. in 10% foetal calf serum (FCS) in PBS, then incubated with mouse monoclonal anti-c-Myc antibody (Autogen bioclear UK Ltd, Wiltshire), diluted according to the manufacturers' instructions in 10% FCS in PBS, for 90 minutes at 4° C. Samples are washed with PBS then incubated with Texas Red labelled anti-mouse IgG antibody (Vector Laboratories, CA), diluted according to the manufacturers' instructions in 10% FCS in PBS, for 60 minutes at 4° C. The cells are washed for a final time in PBS, then wells and gaskets removed. Samples are dried at 22° C., mounted under a coverslip using vectashield mounting medium (Vector Laboratories, CA) and analysed under a fluorescent microscope.

### Example 11

### Protocol for Transduction of Peripheral Blood CD4⁺ T Lymphocytes (Gene Therapy)

Peripheral blood mononuclear cells (PBMCs) from each patient are selected by standard procedure. PBMCs (approximately 10⁸ mononuclear/kg) are taken from the patient by leukapheresis to obtain sufficient cells for infusion. This apheresis product is overlayed onto a Ficoll-Hypaque density gradient and centrifuged to remove any erythrocytes and neutrophils. The harvested PBMCs are depleted of CD8⁺ lymphocytes using for example an anti-CD8⁺ antibody-coated AIS MicroCel-lector™ flasks, thereby leaving a CD4⁺ enriched cell population which will be stimulated with OKT3 (anti-CD3) antibody.

Activated CD4⁺ T cell are grown and transduced in close systems such as the “Peripheral Blood Lymphocyte-MPS” (cellco Cell Max™ artificial capillary system) or alternatively in the gas permeable Lifecell® X-fold™ bags (Nexell Therapeutics Inc) pre-coated with retronectin™ (TaKaRa, Shiga, Japan). For transduction, cells are exposed to GMP-grade viral conditionated medium containing IL-2 (100U/ml) once or twice a day for two or three consecutive days. At the end of the transduction protocol, cells are harvested and re-infused into the patients (up to 10⁶ CD4⁺ T cells/kg).

### Example 12

### Protocol for Transduction of Bone Marrow Repopulating Cells (Gene Therapy)

Bone marrow repopulating cells (such as CD34⁺) are selected and transduced according to standard protocols. Marrow CD34⁺ or alternatively mobilised peripheral CD34⁺ cells are positively selected by an immunomagnetic procedure (CliniMACS, Miltenyi Biotec, Bergish Gladbach, Germany). CD34⁺ enriched cells are cultured in gas-permeable stem cell culture containers Lifecell® X-fold™ bags (Nexell Therapeutics Inc) pre-coated with retronectin™ (TaKaRa, Shiga, Japan) in serum free medium (X-VIVO 10 or CellGro, Biowhittaker Walkerville, Md.) supplemented with cytokines such as stem cell factor (Amgen), IL-3 (Novartis), IL-6 (R&D Systems) and Flt3-L (R&D Systems). For transduction, cells are exposed to GMP-grade viral conditionated medium containing cytokines once or twice a day up to two consecutive days following the activation period. At the end of the transduction protocol, cells are harvested and infused into the patients (approximately 2-4 10⁷ cells/kg).

### Example 13

### General Protocol for HIV Infection of Transduced Cells

To determine whether cells transduced with repressor constructs are restricted with respect to the expression of HIV, cells are infected with the virus and expression of HIV is assayed via expression of p24 viral antigen as well as cell viability.

Jurkat cells transduced with various retroviral vectors and expressing different zinc fingers (3 positive and one negative) or untransduced Jurkat cells are infected with HIV-1 (strains RF, HXB2 or MN) at four different multiplicities of infection (10-fold dilution series). After virus absorption for 2 hours at room temperature, the cells are washed three times and distributed into duplicate wells of a 48 well cell culture plate (1×10⁵ cells per well in 1 ml of culture fluid). 200 μl of culture fluid is removed from each well and replaced with 200%1 of fresh medium daily, from day 3 until day 7. The harvested culture fluid is then assayed at different dilutions to quantitate levels of p24 viral antigen using a commercial ELISA (Abbott). In addition and in parallel, cells are distributed into duplicate wells of a 96 well plate (5×10⁴ cells per well in 200 μl of medium) and incubated for 6 days prior to the addition of XTT to determine cell viability.

For each virus which is tested, the Virus Input (TCID50) is assayed at the various different dilutions of no virus, 1:100, 1:1000, 1:10000 and 1:100000 for each of the following combinations: Jurkat, Jurkat+vector A, Jurkat+vector B Jurkat+vector C and Jurkat+negative vector.

### Example 14

### Inhibition of HIV-1 Replication in Human T-Cells With a Stable Integrated HIV-BA′-KOX Zinc Finger Repressor

Human Jurkat T-cells cultured in RPMI with 10% FCS are transduced with LNL-derived retrovirus that expresses the zinc finger repressor protein pHIVBA′-KOX (see above Example 9. “Delivery of Zinc Fingers to Human Cells Using a Viral Vector”). Seven days after transduction, the infected cells are sorted for expression of the HIV-BA′-KOX zinc finger and a pool of the cells expressing the zinc finger is made, JurkatBA′-KOX. This population is assayed by FACS analysis to verify expression of CD4/CXCR4 coreceptors against a control Jurkat cell line.

JurkatBA′-KOX and a control Jurkat cell line are seeded into 48 well plates at 2.5×10⁴ cells/well and infected with tenfold serial dilutions of the HXB2 strain of HIV-1. 100 μl of virus supernatant is added to the wells and incubated for 3 hours followed by three washes with 1 ml of growth media. 1 ml of growth media is finally added to the cells and the cells are incubated. Daily measurements of soluble p24 antigen are made by ELISA from the culture supernatants for up to seven days. Comparison of the p24 antigen levels between the control and test cell lines shows the inhibition of HIV-1 replication in human T-cells.

### Example 15

### Selection of HSV Promoter Binding Zn Fingers from Libraries in Phage Display System

This and the following Examples describe the construction and properties of zinc fingers directed against sequences present in the HSV promoter.

Two 9 bp sequences (named t, t2 and t4 shown below), spanning the transactivation complex binding region (including TAATGARAT—underlined on IE175k promoter sequence shown below), are chosen as targets for zinc finger factors.

| | |
|---|---|
| −270 | |
| GATCGGGCGGTAATGAGATGCCATG | HSV IE1 75k |
| TAATGAGAT | t2 |
| GATCGGGCG | t4 |

Target sequences are used to screen libraries of randomized 3 zinc finger proteins in a phage display system. Two bipartite GCGG-anchored libraries 12 and 23 (i.e., Lib12 and Lib23 as described above) are used for screening. Library 12 contains randomisations in fingers 1 and 2 while finger 3 is of fixed sequence design to bind GCGG. Library 23 contains randomisations in fingers 3 and 2 while finger 1 is fixed to bind GGCG sequence.

Proteins binding t4 (i.e., 4/3 and 4A) are selected directly from Lib23.

The nucleic acid sequence of Clone 4/3 is as follows:

ATGGCAGAGGAACgcccatatgctTGCCCTGTCGAGTCCTGCGATCGCCG
CTTTTCTCGCTCGGATGAGCTTACCCGCCATATCCGCATCCACACAGGCC
AGAAGCCCTTCCAGTGTCGAATCTGCATGCGTAACTTCAGTCGTAGTGAC
CACCtgaGCACGCACATCCGCACCCACACAGGCGAGAAGCCTTTTGCCTG
TGACATTTGTGGGAGGAaattTGCCACCAACAGCAACCGCATAAAGCATA
CCAAGATACACCTGCGCCAAAAAGATGCGGCC

The amino acid sequence of Clone 4/3 is as follows:

MAEERPYACPVESCDRRFSRSDELTRHIRIHTGQKPFQCRICMRNFSRSD
HLSTHIRTHTGEKPFACDICGRKFATNSNRIKHTKIHLRQKDAA

The nucleic acid sequence of Clone 4A is as follows:

ATGGCAGAGGAACgcccatatgctTGCCCTGTCGAGTCCTGCGATCGCCG
CTTTTCTCGCTCGGATGAGCTTACCCGCCATATCCGCATCCACACAGGCC
AGAAGCCCTTCCAGTGTCGAATCTGCATGCGTAACTTCAGTCGTAGTGAC
CACCtgaGCGAGCACATCCGCACCCACACAGGCGAGAAGCCTTTTGCCTG
TGACATTTGTGGGAGGAaattTGCCACCAACAACAACCGCAAAAAGCATA
CCAAGATACACCTGCGCCAAAAAGATGCGGCC

The nucleic acid sequence of Clone 4A is as follows:

MAEERPYACPVESCDRRFSRSDELTRHIRIHTGQKPFQCRICMRNFSRSD
HLSEHIRTHTGEKPFACDICGRKFATNNNRKKHTKIHLRQKDAA

A combination of phage library selections and rational design is used to engineer a protein which binds target t2 (TAATGAGAT). Initially, a series of clones that bind the sequence TAATGGGCG (containing the TAATG portion of t2) are selected from Lib23. These clones are pooled and subjected to the following manipulations based on rational design (as described in the description above):

(a) F2 amino acid positions −1, 1 and 2 re engineered such that position −1=Gln, position 1=Asp and position 2=Ala;

(b) amino acid positions of F1 are engineered such that position 6=Arg and position 3=Asn. The resulting clones are predicted to bind the sequence TAATGAGCG. This pool of clones comprising these rational modifications is further randomised at positions −1, 1 and 2 and the resulting library of clones is displayed on phage and subjected to selections using t2, i.e TAATGAGAT.

The nucleotide sequence of Clone 7N is as follows:

ATGGCAGAGGAACgcccatatgctTGCCCTGTCGAGTCCTGCGATCGCCG
CTTTTCTACGCGAACTAACCTTACCCGCCATATCCGCATCCACACCAGGC
CAGAAGCCCTTCCAGTGTCGAATCTGCATGCGTAACTTCAGTCAGGACGC
ACACCtgaGCACGCACATCCGCACCCACACAGGCGAGAAGCCTTTTGCCT
GTGACATTTGTGGGAGGAaattTGCCCAGAGCGCCAACCGCAAAACGCAT
ACCAAGATACACCTGCGCCAAAAAGATGCGGCC

The amino acid sequence of Clone 7N is as follows:

MAEERPYACPVESCDRRFSTRTNLTRHIRIHTGQKPFQKPFQCRICMRNF
SQDAHLSTHIRTHTGEKPFACDICGRKFAQSANRKTHTKIHLRQKDAA

Furthermore, six finger constructs were produced from the three finger clones (for example, 6F6 is a finger protein comprising 7N and 4/3, which binds GATCGGGCG g TAATGAGAT).

The nucleic acid sequence of Clone 6F6 is as follows:

ATGGCAGAGGAACgcccatatgctTGCCCTGTCGAGTCCTGCGATCGCCG
CTTTTCTACGCGAACTAACCTTACCCGCCATATCCGCATCCACACAGGCC
AGAAGCCCTTCCAGTGTCGAATCTGCATGCGTAACTTCAGTCAGGACGCA
CACCtgaGCACGCACATCCGCACCCACACAGGCGAGAAGCCTTTTGCCTG
TGACATTTGTGGGAGGAaattTGCCCAGAGCGCCAACCGCAAAACGCATA
CCAAGATACACCTGCGCCAAAAAGATGGCGAACgcccatatgctTGCCCT
GTCGAGTCCTGCGATCGCCGCTTTTCTCGCTCGGATGAGCTTACCCGCCA
TATCCGCATCCACACAGGCCAGAAGCCCTTCCAGTGTCGAATCTGCATGC
GTAACTTCAGTCGTAGTGACCACCtgaGCACGCACATCCGCACCCACACA
GGCGAGAAGCCTTTTGCCTGTGACATTTGTGGGAGGAaattTGCCACCAA
CAGCAACCGCATAAAGCATACCAAGATACACCTGCGCCAAAAAGATGCGG
CCCGGAATTCCACCACACTGGACTAG

The amino acid sequence of Clone 6F6 is as follows:

MAEERPYACPVESCDRRFSTRTNLTRHIRIHTGQKPFQCRICMRNFSQDA
HLSTHIRTHTGEKPFACDICGRKFAQSANRKTHTKIHLRQKDGERPYACP
VESCDRRFSRSDELTRHIRIHTGQKPFQCRICMRNFSRSDHLSTHIRTHT
GEKPEACDICGRKFATNSNRIKHTKIHLRQKDAARNSTTLD

Clone 6F6 is also fused with the KRAB repression domain of KOX to produce 6F6-KOX.

The nucleic acid sequence of 6F6-KOX is as follows:

ATGGCAGAGGAACgcccatatgctTGCCCTGTCGAGTCCTGCGATCGCCG
CTTTTCTACGCGAACTAACCTTACCCGCCATATCCGCATCCACACAGGCC
AGAAGCCCTTCCAGTGTCGAATCTGCATGCGTAACTTCAGTCAGGACGCA
CACCtgaGCACGCACATCCGCACCCACACAGGCGAGAAGCCTTTTGCCTG
TGACATTTGTGGGAGGAaattTGCCCAGAGCGCCAACCGCAAAACGCATA
CCAAGATACACCTGCGCCAAAAAGATGGCGAACgcccatatgctTGCCCT
GTCGAGTCCTGCGATCGCCGCTTTTCTCGCTCGGATGAGCTTACCCGCCA
TATCCGCATCCACACAGGCCAGAAGCCCTTCCAGTGTCGAATCTGCATGC
GTAACTTCAGTCGTAGTGACCACCtgaGCACGCACATCCGCACCCACACA
GGCGAGAAGCCTTTTGCCTGTGACATTTGTGGGAGGAaattTGCCACCAA
CAGCAACCGCATAAAGCATACCAAGATACACCTGCGCCAAAAAGATGCGG
CCcggaattccggccaaaaaagagaaaaggtcgacggcggtggtgctttg
tctcctcagcactctgctgtcactcaaggaagtatcactggtgaccttca
aggatgtatttgtggacttcaccagggaggagtggaagctgctggacact
gctcagcagatcgtgtacagaaatgtgatgctggagaactataagaacct
ggtttccttgggttatcagcttactaagccagatgtgatcctccggttgg
agaagggagaagagccctggctggtggagagagaaattcaccaagagacc
catcctgattcagagactgcatttgaaatcaaatcatcagttgaacaaaa
acttatttctgaagatctgtaa

The amino acid sequence of 6F6-KOX is as follows:

MAEERPYACPVESCDRRFSTRTNLTRHIRIHTGQKPFQCRICMRNFSQDA
HLSTHIRTHTGEKPFACDICGRKFAQSANRKTHTKIHLRQKDGERPYACP
VESCDRRFSRSDELTRHIRIHTGQKPFQCRICMRNFSRSDHLSTHIRTHT
GEKPFACDICGRKFATNSNRIKHTKIHLRQKDAARNSGPKKRKVDGGGAL
SPQHSAVTQGSIIKNKEGMDAKSLTAWSRTLVTFKDVFVDFTREEWKLLD
TAQQIVYRNVMLENYKNLVSLGYQLTKPDVILRLEKGEEPWLVEREIHQE
THPDSETAFEIKSSVEQKLISELD*

Zinc finger constructs are cloned into vectors for further manipulation. These are described below.

Primers Used for PCR Cloning

| | |
|---|---|
| 4AFOR: | CTG CTC TAG AGC GCC GCC.ATG GCA GAG |
| | GAA CGC; |
| HIV13Rev: | TCC GGG ATC CCG CGG AAT TCC GGG CCG |
| | CAT CTT TTT GGC GCA GGT G; |
| HIV13For: | CTC TAG AGC GCC GCC ATG GCG GAA GAG |
| | AGG CCC; |
| NSFUS2: | GAA ACG CCC ATA TGC TTG CCC TGT C; |
| RevlinGly: | CAG GGC AAG CAT ATG GGC GTT C GCC ATC |
| | TTT TTG GCG CAG GTG TAT CTT GG; |
| FOR2: | GA CAG AAG GAC GCG GCC ACG CGT CCA |
| | AAA AAG AAG AGA AAG GTC; |
| REV2: | CGC GGA TCC TTA CAG ATC TTC TTC AGA |
| | AAT AAG TTT TTG TTC AAC TGA TGA TTT |
| | GAT TTC AAA TGC; |
| 6F6HIND FOR: | CTA CGT AAG CTT GCG CCG CCA TGG CAG |
| | AGG AAC G; |
| KOX/VP16REV: | GCT CGG ATC CTT ACA GAT CTT CTT CAG A |

Plasmids

pc413 is an expression plasmid based on pcDNA 3.1 (−) (Invitrogen) that expresses the zinc finger protein Clone 4/3. The sequence encoding the 3-finger domain (described above) is amplified from the phage clone 4/3 using 4AFOR primer and HIV13Rev primer, and cloned into XbaI and EcoRI sites of pcDNA3.1 (−). The TAG sequence present 7 codons downstream from EcoRI site in the MCS serves as a stop codon.

pc4A is an expression plasmid based on pcDNA 3.1 (−) that expresses the zinc finger protein Clone 4A. The sequence encoding the 3-finger domain (described above) is amplified from the phage clone 4A using 4AFOR primer and HIV13Rev primer, and cloned into XbaI and EcoRI sites of pcDNA3.1 (−). The TAG sequence present 7 codons downstream from EcoRI site in the MCS serves as a stop codon

pc7N is an expression plasmid based on pcDNA 3.1 (−) that expresses the zinc finger protein Clone 7N. The sequence encoding the 3-finger domain (described above) is amplified from the phage clone 7N using 4AFOR primer and HIV13Rev primer, and cloned into XbaI and EcoRI sites of pcDNA3.1 (−). The TAG sequence present 7 codons downstream from EcoRI site in the MCS serves as a stop codon

pc4A-KOX is a plasmid based on pcDNA 3.1 (−), which expresses a fusion protein comprising the DNA binding domain of Clone 4A and the repression domain from KOX protein (i.e., 4A-KOX). A DNA fragment corresponding to the 3-finger domain is amplified by PCR from the phage clone 4A as above and joined with regions coding for NLS, KRAB repression domain from KOX and c-myc epitope, generated by PCR amplification.

pc4/3-KOX is a plasmid based on pcDNA 3.1 (−), which expresses 4/3-KOX fusion protein, i.e., a DNA binding domain of Clone 4/3 together with the KOX repression domain. A DNA fragment corresponding to the 3-finger domain is amplified by PCR from the phage clone 4/3 as above and joined with regions coding for NLS, KRAB repression domain from KOX and c-myc epitope, generated by PCR amplification (as above).

pcHIV3-KOX is a plasmid based on pcDNA 3.1 (−), which expresses HIV3-KOX fusion protein, i.e., Clone HIV-C of Table 1 fused with the KOX repression domain. It is used as a negative control in HSV-1 infections. A DNA fragment corresponding to a 3-finger domain selected to recognize DNA sequence from the HIV LTR (GAT GCT GCA) is amplified by PCR from selected phage clone (HIV-C) as above and joined with regions coding for NLS, KRAB repression domain from KOX and c-myc epitope, generated by PCR amplification (as above).

pc6F6 is a protein expression plasmid based on pcDNA 3.1 (−) which expresses 6F6, a six finger DNA binding domain comprising a fusion between three finger clones 7N and 4/3. DNA fragments corresponding to 3-finger domains are PCR amplified directly from phage clones 7N and 4/3 selected to bind t2 and t4 respectively (described above). Primers 4AFOR and RevlinGly are used to amplify the 7N portion of the protein and primers HIV13Rev and NCFUS2 are used to amplify the 4/3 portion The PCR products are mixed and subjected to a second round of amplification using only an external pair of primers 4AFOR and HIV13REV. The resulting product (sequence shown above) is cloned into the XbaI and EcoRI sites of pcDNA3. (−).

pc6F6-KOX is a plasmid expressing a fusion protein (6F6-KOX) comprising the six finger DNA binding domain from 6F6 and the KRAB repression domain of KOX. It is constructed by swapping the 4A 3-finger DNA binding domain in pc4A-KOX with the 6F6 domain from pc6F6.

pFRT6F6 To construct this vector, the 6F6-KOX coding sequence is PCR amplified from pc6F6-KOX using 6F6HIND FOR and KOX/VP16Rev primers and cloned into the HindIII and BamHI sites of pcDNA5/FRT (Invitrogen).

p6F6-KOX-TRACER is based on pTRACER-CMV/Bsd (Invitrogen) and expresses 6F6-KOX from the CMV promoter and Cycle3 GFP-blasticidin from the EF-1 promoter. This plasmid is constructed by extracting a NheI-NotI fragment (which contains the entire 6F6-KOX sequence with fragments of polylinker) from pFRT6F6 and cloning it into the NheI and NotI sites of pTracer CMV/Bsd (Invitrogen)

pPO13 is a reporter plasmid containing the entire HSV IE175k promoter region (−380 to +30) fused to a CAT reporter gene (donated by P.O'Hare)

pCMV-VP16 (RG50) is a plasmid expressing full length HSV-I VP16 protein from the CMV IE promoter (donated by P.O'Hare)

Organisms

Bacterial strains: TG1; virus strains: HSV-1 strain 17 (donated by A. Minson); cell lines: HeLa, COS-1, HeLa T-REX (Invitrogen).

### Example 16

### Protocols for Zinc Finger Binding Assays

Phage Display ELISA Assay

A standard phage ELISA method is used to evaluate the specificity and Kd of 3-finger proteins that bind to HSV sequences. Binding of the 3 finger proteins displayed on phage is tested against closely related targets (to test specificity) as well as against serial dilutions of their 9 bp target sites ranging from 0.125 to 32 nM. Phage displaying the three finger domain from Zif268 is used as a control in these experiments (Kd about 1-2 nM when bound to its optimal DNA target 5′-GCGTGGGCG-3′).

Gel Retardation (Bandshift) Assays

Three finger proteins and their derivatives are expressed in vitro (TNT system, Promega) mixed with radioactively labeled target DNA and subjected to electrophoresis in native gels. Binding studies are performed using an excess of protein (tested in serial 5 fold dilutions) and with constant amounts of DNA (0.1 nM). DNA binding reactions contain the appropriate zinc-finger peptide, binding site and 1 μg competitor DNA (Holy dI-dC) in a total volume of 10 μl, which contains: 20 mM Bis-tris propane (pH 7.0), 100 mM NaCl, 5 mM MgCl₂, 50 PM ZnCl₂, 5 mM DTT, 0.1 mg/ml BSA, 0.1% Nonidet P40. Incubations are performed at room temperature for 1 hour.

Binding of zinc finger proteins is assayed in the presence and absence of regulatory domains fused to the C-terminus. The 6-finger construct which binds to the IE175 promoter (6F6) is also tested on related sites e.g. those present in the IE68k promoter region (contains 3 mismatches in the 19 bp target), the IE 11 Ok promoter region (8 mismatches in 19 bp target) and the human H2B promoter normally activated by Oct-1 (11 mimatches)

The sequences of molecular probes used for gel retardation assays are as follow:

| | |
|---|---|
| T24: | CCG CCG GAT CGG GCG G TAA TGA GAT GCC ATG |
| H2B: | ATA GAA TCG CTT ATG C AAA TAA GGT GAA GA |
| 68K: | CTT CCC GGT TCG GCG G TAA TGA GAT ACG AG |
| IE110: | TGG GTT CCG GGT ATG G TAA TGA GTT TCT TC |

Transfections of Mammalian Cell Lines

Zinc finger constructs are also co-transfected to HeLa or COS-1 cells along with CAT reporter gene containing target DNA site (as described above). The cells are harvested at 40-48 h post transfection and assayed for the levels of CAT enzyme using CAT ELISA Kit (Roche) according to manufacturer instructions.

Transient transfections of COS-1 and HeLa cells are performed using FuGene (Roche) and CsCl purified DNA, according to the manufacturer's instructions. Cells are plated the day before transfection into cluster dishes (6×35 mm) at 2×10⁵ cells per well and the medium is changed directly before transfection. L-2 μg of total DNA is used, equalized in all cases by addition of pUC19 carrier DNA. For CAT assays, pcDNA 3.1 (−) vector is added when required to equalize total levels of CMV promoter input.

HSV-1 Infections of Cells Transiently Transfected with 6F6-KOX Constructs

Subconfluent COS-1 cells are transfected with pc6F6-KOX using FuGene (as described above) to a minimum efficiency of transfection of 30%, and infected with 0.01-0.1 pfu/cell of HSV-1 strain 17 at 40 h post transfection. Infection is carried out in 24-well or 6-well cluster tissue culture dishes in 300 or 1000 μl of medium (DMEM+2% FCS) respectively, at 37 degrees C. for 1 h (no shaking), followed by changing medium and incubation at 37 degrees C. Infected cells are washed in PBS and harvested in 100 or 300 μl (from 24 or 6-well cluster dish, respectively) of hot SDS-loading buffer and analyzed by Western blots.

To ensure that all the cells intended for infection express 6F6-KOX, COS-1 cells are transfected with p6F6-KOX-TRACER and at 24 h post transfection cells are subjected to FACS sorting using GFP as a tracer. Prior to FACS sorting transfected cells are washed twice in PBS and harvested in trypsin and neutalised with DMEM with 10%FCS, spun down at 1500 g 5 min, resuspended in PBS+propidium iodide (0.005 ng/ml) and strained through a cell strainer. Only cells positive for GFP and negative for propidium iodide are selected, spun down, resuspended in fresh medium and replated in either 6-well or 24-well plates at desired densities. The cells are infected, as above, with HSV-1 at 16-24 hours after re-plating and harvested at different time points post infection.

To estimate a number of HSV-1 particles released at different times post infection, medium from cells infected in 24-well cluster dish (300 μl) is collected and used in a standard serial dilution plaque assay.

Western Blots of Total Cell Lysates

Adherent mammalian cells intended for Western blot analysis are washed twice in PBS and lysed in 100 or 300%1 of hot SDS-loading buffer directly on the plate (6 or 24-well cluster dish, respectively), harvested and boiled for 5 min. Samples are sonicated and boiled again directly before being subjected to SDS-PAGE. Usually 50 μl samples are applied per well. Proteins are blotted onto nitrocellulose, probed with relevant antibodies and detected using the ECL detection system according to the manufacturer's instructions (Amersham). The c-myc epitope-tagged proteins are detected with monoclonal antibody 9E10 (Santa Cruz) used at a dilution of 1:200, HSV-1 VP16 is detected with monoclonal antibody LP1 (donated by A. Minson) used at a dilution of 1:100, HSV IE110k is detected with rabbit polyclonal antibody r191 (donated by R. Everett) and HSV IE175k is detected with monoclonal antibody 10176 (donated by R. Everett) used at a dilution of 1:5000. The same membrane is stripped and re-blotted up to 5 times.

### Example 17

### Analysis of 3-Finger Protein Selected to Bind T4 (GATCGGGCG) and T2 (TAATGAGAT)

The 3-finger proteins selected to bind the DNA sequences t4 (GATCGGGCG) and t2 (TAATGAGAT) are initially screened by phage ELISA assays against related targets. The phage displayed clones 4A, 4/3 and 7N selected to recognize t4 (4/3 and 4A) and t2 (7N) are tested against serial dilutions of their target site (FIG. 10) and compared directly with Zif268 displayed on phage. All of the clones tested −4A, 4/3 and 7N exhibited apparent Kds comparable with Zif268 (about 1 nM), with 7N being the weakest binder.

The 4/3 protein has slightly higher affinity (about 2 fold) for the t4 site than 4A; however it is marginally less discriminative when tested against closely related sites. 4A and 4/3 are also tested in gel retardation assays with a DNA fragment containing the t4 site (T24). Data from these experiments agrees with the ELISA results where 4/3 is found to be a stronger binder than 4A. The gel retardation studies of 7N confirm its strong affinity for the t2 site. When tested in parallel with 4/3 protein using a DNA probe containing both t2 and t4 sites (T24), both of the 3 finger proteins shown roughly similar apparent Kd.

To perform in vivo analysis, the 3-finger domains of 4A and 4/3 are fused to the KRAB repression domain from KOX, the NLS from SV40 large T antigen, and a c-myc epitope tag and are cloned into a eukaryotic expression vector (resulting in p4A-KOX and p4/3-KOX). The above constructs are tested in COS and HeLa cells for repression of an IE175k-CAT reporter construct in the presence of full length VP16 (added as an additional plasmid to transfection, in order to mimic gene activation during HSV infection). High levels of activation (about 30 fold) are elicited by VP16 alone suggesting that IE175k promoter is active and responsive. No significant repression by either 4A-KOX or 4/3-KOX is observed, despite the presence of recombinant proteins in the cells (confirmed by Western blots and immunofluorescence).

From these results it can be concluded that the 3-finger protein does not bind to the promoter (which contains only a single t4 site) with high enough affinity to cause a strong effect on gene expression and longer arrays of zinc fingers are needed.

### Example 18

### Analysis 6-Finger Protein Binding T4+T2 (GATCGGGCGGTAATGAGAT)

In an attempt to create a strong binder (capable of in vivo HSV inhibition via binding to the complete t4+t2 site), the 4/3 and 7N 3-finger proteins are fused using the amino acid sequence QKDGERP as a linker to form a 6-finger protein (6F6). The resulting 6-finger protein (6F6) is capable of binding one of the two TAATGARAT sequences (+adjacent region) present in the IE175k promoter (position −230 in respect to the start of transcription).

Predicted contacts between the DNA target sequences t4 and t2 and 3-finger domains 4/3 and 7N are shown on FIG. 11

When tested in gel retardation assays 6F6 shows at least 25 fold greater affinity for its composite DNA site than any of its 3-finger components alone (i.e., 4/3 or 7N) (FIG. 12).

When tested on related sites (FIG. 13) e.g. the IE68k promoter region (containing 3 mismatches in 19 bp target), the IE110k promoter region containing octa+motif (8 mismatches in 19 bp target) and the human H2B promoter normally activated by Oct1 (11 mismatches), 6F6 shows almost no affinity for these sites within the concentration range tested while e.g. 7N binds the IE68k promoter containing the intact t2 site as well as the IE110k promoter.

The 6-finger protein has therefore both higher affinity and higher specificity than 3-finger proteins.

The 6F6 peptide is subsequently fused to the KRAB repression domain from KOX, equipped with the NLS from the SV40 large T antigen and c-myc epitope tag and tested in vivo. Prior to CAT assay experiments the fusion proteins are subjected to bandshift assays, which reveal that the presence of the additional domains does not significantly alter 6F6 binding affinity.

In vivo analysis of 6F6 focussed on repression studies in which expression of CAT is driven by the IE175k promoter, activated with wild type VP16 and repressed with different doses of 6F6-KOX. In all the cell lines used (COS and HeLa) 6F6-KOX has a clear inhibitory effect on activated expression from the IE175k promoter and the degree of repression is found to depend on the amount of 6F6-KOX. The repression is over 90% with the highest dose of 6F6-KOX plasmid used (FIG. 14).

The 6F6 alone (no repression domain) is also found to partly inhibit CAT expression and it confirms our initial assumption that the zinc finger protein competes with VP16 for binding to TAATGAGAT, and repression by 6F6-KOX is partly due to the competition and partly due to the repressive action of KRAB. In the presence of KRAB the repression effect is about 3-fold greater. The conclusion is that 6F6-KOX is capable of inhibiting transcription from the IE175k promoter when used in the CAT reporter system.

### Example 19

### Inhibition of HSV-1 Infection by 6F6-KOX

Initial experiments with HSV-1 are carried out in transient transfection system. The viral gene expression is monitored using Western blots during the course of infection in the presence and absence of 6F6-KOX (FIG. 15). For control experiments a zinc finger construct selected to bind an unrelated DNA sequence (HIV3-KOX, which comprises Clone HIV-C of Table 1 fused to a KOX repression domain) is used. A significant delay in appearance of all classes of HSV-1 proteins (including IE and late) is observed when infection is carried out in the presence of 6F6-KOX when compared with infection in the cells expressing control the fusion protein (HIV3-KOX). Taking into account that only about 30-35% of the cells infected with HSV in this type of experiment are expressing recombinant proteins (due to the limitations of transfection), the inhibitory effect of 6F6-KOX on HSV-1 infection is significant.

To enrich the population of 6F6-KOX positive cells in the transiently transfected pool, the p6F6-KOX-TRACER vector is employed and transfected cells are subjected to FACS sorting using GFP as a tracer. Cells selected by this type of procedure are used for HSV-1 infection and virus titre analysis (FIG. 16). The total number of infectious viral particles released by 6F6-KOX positive cells is found to be 10 fold lower than amount of virus released by control cells (which express GFP alone).

This level of virus inhibition in single-step growth experiment is comparable with the results obtained with mutant viruses containing insertions or deletions in the ORF coding for the IE110k gene. Specifically, in these experiments a 10-100 fold reduction in p.f.u. yields (depending on the mutated region) is observed. (Everett, R. D. Construction and characterization of herpes simplex virus type I mutants with defined lesions in immediate early gene 1. J. Gen. Virol 70, 1185-1202(1989))

In summary, we show that nucleic acid binding polypeptides comprising zinc fingers can be selected and/or designed against viral sequences, in particular viral promoter sequences. Such zinc fingers are shown to bind to their targets with high specificity and affinity both in vitro and in vivo, and are capable of repressing and otherwise modulating gene expression of reporters, as well as the native viral proteins.

### REFERENCES

1. Choo, Y., Sanchez-Garcia, I. & Klug, A. In vivo repression by a site-specific DNA-binding protein designed against an oncogenic sequence. Nature 372, 642-645 (1994).

2. Greisman, H. A. & Pabo, C, O. A general strategy for selecting high-affinity zinc finger proteins for diverse DNA target sites. Science 275, 657-661 (1997).

3. Klug, A. & Rhodes, D. ‘Zinc fingers’: a novel protein motif for nucleic acid recognition. Trends Biochem. Sci. 12, 464469 (1987).

4. Choo, Y. & Klug, A. Designing DNA-binding proteins on the surface of filamentous phage. Curr. Opin Biotech 6,431-436 (1995).

5. Miller, J., McLachlan, A. D. & Klug, A. Repetitive zinc-binding domains in the protein transcription factor IIIA from Xenopus oocytes. EMBO J 4, 1609-1614 (1985).

6. Pavletich, N. P. & Pabo, C, O. Zinc finger-DNA recognition: Crystal structure of a Zif268-DNA complex at 2.1 Å. Science 252, 809-817 (1991).

7. Rebar, E. J. & Pabo, C, O. Zinc Finger Phage: Affinity Selection of Fingers with New DNA-Binding Specificities. Science 263, 671-673 (1994).

8. Jamieson, A. C., Kim, S.-H. & Wells, 3. A. In vitro selection of zinc fingers with altered DNA-binding specificity. Biochemistry 33, 5689-5695 (1994).

9. Choo, Y. & Klug, A. Toward a code for the interactions of zinc fingers with DNA: Selection of randomised zinc fingers displayed on phage. Proc. Natl. Acad. Sci. U.S.A. 91, 11163-11167 (1994).

10. Wu, H., Yang, W.-P. & Barbas III, C. F. Building zinc fingers by selection: Toward a therapeutic application. Proc. Natl. Acad. Sci. USA 92, 344-348 (1995).

11. Isalan, M., Klug, A. & Choo, Y. Comprehensive DNA recognition through concerted interactions from adjacent zinc fingers. Biochemistry 37, 12026-12033 (1998).

12. Choo, Y. Recognition of DNA methylation by zinc fingers. Nature Struct. Biol. 5, 264-265 (1998).

13. Segal, D. J., Dreier, B., Beerli, R. R. & Barbas, C. F. Toward controlling gene expression at will: selection and design of zinc finger domains recognising each of the 5′-GNN-3′ DNA target sequences. Proc. Natl. Acad. Sci. USA 96, 2758-2763 (1999).

14. Isalan, M. & Choo, Y. Engineered zinc finger proteins that recognise DNA modification by HaeIII and HBhaI methyltransferase enzymes. J Mol Biol 295, 471477 (2000).

15. Beerli, R. R., Dreier, B. & Barbas, C. F. Positive and negative regulation of endogenous genes by designed transcription factors. Proc Natl Acad Sci Early Edition (2000).

16. Isalan, M. D. & Choo, Y. Engineering protein-nucleic acid recognition. Curr Opin Struct Biol 10, Issue 4, in press (2000).

17. Wolfe, S. A., Greisman, H. A., Ramm, E. I. & Pabo, C, O. Analysis of zinc fingers optimised via phage display: evaluating the utility of a recognition code. J. Mol. Biol. 285, 1917-1934 (1999).

18. Isalan, M., Choo, Y. & Klug, A. Synergy between adjacent zinc fingers in sequence-specific DNA recognition. Proc Natl Acad Sci 94, 5617-5621 (1997).

19. Christy, B. A., Lau, L. F. & Nathans, D. A gene activated in mouse 3T3 cells by serum growth factors encodes a protein with “zinc finger” sequences. Proc. Natl. Acad Sci. USA 85, 7857-7861 (1988).

20. Choo, Y. & Klug, A. Selection of DNA binding sites for zinc fingers using rationally randomised DNA reveals coded interactions. Proc. Natl. Acad. Sci. U.S.A. 91, 11168-11172 (1994).

21. Choo, Y. & Klug, A. Physical basis of a protein-DNA recognition code. Curr. Opin. Str. Biol. 7, 117-125 (1997).

22. Elrod-Erickson, M., Rould, M. A., Nekludova, L. & Pabo, C, O. Zif268 protein-DNA complex refined at 1.6A: a model system for understanding zinc finger interactions. Structure 4, 1171-1180 (1996).

Each of the applications and patents mentioned above, and each document cited or referenced in each of the foregoing applications and patents, including during the prosecution of each of the foregoing applications and patents (“application cited documents”) and any manufacturer's instructions or catalogues for any products cited or mentioned in each of the foregoing applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference. In particular, we hereby incorporate by reference International Patent Application Numbers PCT/GB00/02080, PCT/GB00/02071, PCT/GB00/03765, United Kingdom Patent Application Numbers GB0001582.6, GB0001578.4, and GB9912635.1 as well as U.S. Ser. No. 09/478,513.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

On page 3, please replace the paragraph from line 12 to line 27 with the following amended paragraph:

FIG. 2. Composition of the ‘bipartite’ library. (a) DNA recognition by the two zinc finger master libraries, Lib12 and Lib23. The libraries are based on the three-finger DNA-binding domain of Zif268 and the putative binding scheme is based on the crystal structure of the wild-type domain in complex with DNA (6, 22). The DNA-binding positions of each zinc finger are numbered and randomised residues in the two libraries are circled. Broken arrows denote possible DNA contacts from Lib12 to bases H′IJKLM and from Lib23 to bases MNOPQ. Solid arrows show DNA contacts from those regions of the two libraries that carry the wild-type Zif268 amino acid sequence, as observed in the crystal structure. The wild-type portion of each library target site (white boxes) determines the register of the zinc finger-DNA interactions, such that the selected portions of the two libraries can be recombined to recognise the composite site H′IJKLMNOPQ. (b) Amino acid composition (SEQ ID NO: 1) of the randomised DNA-binding positions on the α-helix of each zinc finger. A subset of the 20 amino acids is included in each DNA-binding position. Note that positions 4 and 5 of F2 (LS) are specified by the codons CTG AGC, which contain the recognition site of the restriction enzyme DdeI (underlined), used as a breakpoint to recombine the products of the two libraries.

On page 4, please replace the paragraph from line 18 to line 27 with the following amended paragraph:

FIG. 4. Binding sites of zinc finger DNA binding doamins selected to recognise the HIV-1 LTR. Shown is the 9 kbp HIV-1 genome encoding the gag pol env genes and the 5′ and 3′ long terminal repeats (LTR). These genes are transcribed from a single promoter in the 5′ LTR, the DNA sequence (SEQ ID NO: 2) of which is shown in detail. This is the sequence as reported by Jones and Peterlin Annu. Rev. Biochem. 63:717-743 (1994). The DNA bases in the sequence are numbered relative to the transcription start site (+1). Highlighted above the sequence are the binding sites for the human transcription factors NF-kB and SP1. Highlighted below the sequence are the sites targeted by exemplary zinc finger DNA binding domains selected by the bipartite selection strategy as described herein (HIV-A, HIV-A′, HIV-B to HIV-G).

On page 6, please replace the paragraph from line 6 to line 8 with the following amended paragraph:

FIG. 9. Mechanism of activation of HSV-1 IE genes by VP16 interaction with TAATGARAT elements. Two types of TAATGARAT sites—octa+ (SEQ ID NO: 3) and octa− are shown on IE175k and IE110k promoters respectively.

On page 18, please replace the paragraph from line 13 to line 14 with the following amended paragraph:

In general, a preferred zinc finger framework has the structure (SEQ ID NO: 4):

X₀₋₂ C X₁₋₅ C X₉₋₁₄ H X₃₋₆ H/C

On page 18, please replace the paragraph from line 17 to line 19 with the following amended paragraph:

The above framework may be further refined to include the structure (SEQ ID NO 5):

| (A′) X₀₋₂ C X₁₋₅ C X₂₋₇ | X X X X X X X H X₃₋₆ ^{H}/_{C} |
|---|---|
| | −1 1 2 3 4 5 6 7 |

On page 18, please replace the paragraph from line 20 to line 21 with the following amended paragraph:

In a preferred aspect of the present invention, zinc finger nucleic acid binding motifs may be represented as motifs having the following primary structure (SEQ ID NO: 6):

On page 21, please replace the paragraph from line 19 to line 23 with the following amended paragraph:

Consensus zinc finger structures may be prepared by comparing the sequences of known zinc fingers, irrespective of whether their binding domain is known. Preferably, the consensus structure is selected from the group consisting of the consensus structure P Y K C P E C G K S F S Q K S D L V K H Q R T H T (SEQ ID NO: 7), and the consensus structure P Y K C S E C G K A F S Q K S N L T R H Q R I H T (SEQ ID NO: 8).

On page 26, please replace the paragraph from line 4 to line 14 with the following amended paragraph:

By “linker sequence” we mean an amino acid sequence that links together two nucleic acid binding modules. For example, in a “wild type” zinc finger protein, the linker sequence is the amino acid sequence lacking secondary structure which lies between the last residue of the α-helix in a zinc finger and the first residue of the β-sheet in the next zinc finger. The linker sequence therefore joins together two zinc fingers. Typically, the last amino acid in a zinc finger is a threonine residue, which caps the α-helix of the zinc finger, while a tyrosine/phenylalanine or another hydrophobic residue is the first amino acid of the following zinc finger. Accordingly, in a “wild type” zinc finger, glycine is the first residue in the linker, and proline is the last residue of the linker. Thus, for example, in the Zif268 construct, the linker sequence is G(E/Q)(K/R)P (SEQ ID NO: 9-12).

On page 26, please replace the paragraph from line 15 to line 22 with the following amended paragraph:

A “flexible” linker is an amino acid sequence which does not have a fixed structure (secondary or tertiary structure) in solution. Such a flexible linker is therefore free to adopt a variety of conformations. An example of a flexible linker is the canonical linker sequence GERP (SEQ ID NO: 9)/GEKP (SEQ ID NO: 10)/GQRP (SEQ ID NO: 11)/GQKP (SEQ ID NO: 12). Flexible linkers are also disclosed in WO99/45132 (Kim and Pabo). By “structured linker” we mean an amino acid sequence which adopts a relatively well-defined conformation when in solution. Structured linkers are therefore those which have a particular secondary and/or tertiary structure in solution.

On page 27, please replace the paragraph from line 14 to line 25 with the following amended paragraph:

Once the length of the amino acid sequence has been selected, the sequence of the linker may be selected, for example by phage display technology (see for example U.S. Pat. No. 5,260,203) or using naturally occurring or synthetic linker sequences as a scaffold (for example, GQKP (SEQ ID NO: 12) and GEKP (SEQ ID NO: 10), see Liu et al., 1997, Proc. Natl. Acad. Sci. USA 94, 5525-5530 and Whitlow et al., 1991, Methods: A Companion to Methods in Enzymology 2: 97-105). The linker sequence may be provided by insertion of one or more amino acid residues into an existing linker sequence of the nucleic acid binding polypeptide. The inserted residues may include glycine and/or serine residues. Preferably, the existing linker sequence is a canonical linker sequence selected from GEKP (SEQ ID NO: 10), GERP (SEQ ID NO: 9), GQKP (SEQ ID NO: 12) and GQRP (SEQ ID NO: 11). More preferably, each of the linker sequences comprises a sequence selected from GGEKP (SEQ ID NO: 13), GGQKP (SEQ ID NO: 14), GGSGEKP (SEQ ID NO: 15), GGSGQKP (SEQ ID NO: 16), GGSGGSGEKP (SEQ ID NO: 17), and GGSGGSGQKP (SEQ ID NO: 18).

On pages 34-36, please replace the paragraph from line 4 on page 34 to page 36 with the following amended paragraph:

In a preferred embodiment of the invention, a nucleic acid binding polypeptide capable of binding a human immunodeficiency virus nucleotide sequence comprises one or more of the following sequences:

| | | |
|---|---|---|
| SEQ ID NO: | Sequence | Name |

| | | |
|---|---|---|
| 19 | X₀₋₂ C X1-5 C X₂₋₇ R S D E L T R H X₃₋₆ ^{H}/_{C} | HIV-A F1 |
| 20 | X₀₋₂ C X1-5 C X₂₋₇ R S D N L S T H X₃₋₆ ^{H}/_{C} | HIV-A F2 |
| 21 | X₀₋₂ C X1-5 C X₂₋₇ R R D H R T T H X₃₋₆ ^{H}/_{C} | HIV-A F3 |
| 22 | X₀₋₂ C X1-5 C X₂₋₇ R S D V L T R H X₃₋₆ ^{H}/_{C} | HIV-A′ F1 |
| 23 | X₀₋₂ C X1-5 C X₂₋₇ R S D H L T T H X₃₋₆ ^{H}/_{C} | HIV-A′ F2 |
| 24 | X₀₋₂ C X1-5 C X₂₋₇ D Y S V R K R H X₃₋₆ ^{H}/_{C} | HIV-A′ F3 |
| 25 | X₀₋₂ C X1-5 C X₂₋₇ D S A H L T R H X₃₋₆ ^{H}/_{C} | HIV-B F1 |
| 26 | X₀₋₂ C X1-5 C X₂₋₇ R S D H L S T H X₃₋₆ ^{H}/_{C} | HIV-B F2 |
| 27 | X₀₋₂ C X1-5 C X₂₋₇ D S A N R T K H X₃₋₆ ^{H}/_{C} | HIV-B F3 |
| 28 | X₀₋₂ C X1-5 C X₂₋₇ A S A D L T R H X₃₋₆ ^{H}/_{C} | HIV-C F1 |
| 29 | X₀₋₂ C X1-5 C X₂₋₇ N R S D L S R H X₃₋₆ ^{H}/_{C} | HIV-C F2 |
| 30 | X₀₋₂ C X1-5 C X₂₋₇ T S S N R K K H X₃₋₆ ^{H}/_{C} | HIV-C F3 |
| 31 | X₀₋₂ C X1-5 C X₂₋₇ H S S D L T R H X₃₋₆ ^{H}/_{C} | HIV-D F1 |
| 32 | X₀₋₂ C X1-5 C X₂₋₇ Q S S D L S K H X₃₋₆ ^{H}/_{C} | HIV-D F2 |
| 33 | X₀₋₂ C X1-5 C X₂₋₇ Q N A T R K R H X₃₋₆ ^{H}/_{C} | HIV-D F3 |
| 34 | X₀₋₂ C X1-5 C X₂₋₇ D S S S L T K H X₃₋₆ ^{H}/_{C} | HIV-E F1 |
| 35 | X₀₋₂ C X1-5 C X₂₋₇ Q S A H L S T H X₃₋₆ ^{H}/_{C} | HIV-E F2 |
| 36 | X₀₋₂ C X1-5 C X₂₋₇ D S S S R T K H X₃₋₆ ^{H}/_{C} | HIV-E F3 |
| 37 | X₀₋₂ C X1-5 C X₂₋₇ A S D D L T Q H X₃₋₆ ^{H}/_{C} | HIV-F F1 |
| 38 | X₀₋₂ C X1-5 C X₂₋₇ R S S D L S R H X₃₋₆ ^{H}/_{C} | HIV-F F2 |
| 39 | X₀₋₂ C X1-5 C X₂₋₇ Q S A H R T K H X₃₋₆ ^{H}/_{C} | HIV-F F3 |
| 40 | X₀₋₂ C X1-5 C X₂₋₇ R S D A L I Q H X₃₋₆ ^{H}/_{C} | HIV-G F1 |
| 41 | X₀₋₂ C X1-5 C X₂₋₇ D R A N L S T H X₃₋₆ ^{H}/_{C} | HIV-G F2 |
| 42 | X₀₋₂ C X1-5 C X₂₋₇ A S S T R T K H X₃₋₆ ^{H}/_{C} | HIV-G F3 |
| 43 | X₀₋₂ C X₁₋₅ C X₂₋₇ R S D E L T R H X₃₋₆ ^{H}/_{C}- | HIV-A |
| | linker-X₀₋₂ C X₁₋₅ C X₂₋₇ R S D N L S T H | |
| | X₃₋₆ ^{H}/_{C}-linker-X₀₋₂ C X₁₋₅ C X₂₋₇ R R D H R | |
| | T T H X₃₋₆ ^{H}/_{C} | |
| 44 | X₀₋₂ C X₁₋₅ C X₂₋₇ D S A H L T R H X₃₋₆ ^{H}/_{C}- | HIV-A′ |
| | linker -X₀₋₂ C X₁₋₅ C X₂₋₇ R S D H L S T H | |
| | X₃₋₆ ^{H}/_{C}-linker-X₀₋₂ C X₁₋₅ C X₂₋₇ D S A N R | |
| | T K H X₃₋₆ ^{H}/_{C} | |
| 45 | X₀₋₂ C X₁₋₅ C X₂₋₇ R S D V L T R H X₃₋₆ ^{H}/_{C}- | HIV-B |
| | linker-X₀₋₂ C X₁₋₅ C X₂₋₇ R S D H L T T H | |
| | X₃₋₆ ^{H}/_{C}-linker-X₀₋₂ C X₁₋₅ C X₂₋₇ D Y S V R | |
| | K R H X₃₋₆ ^{H}/_{C} | |
| 46 | MAERPYACPVESCDRRFSRSDVLTRHIRIHTGQKPFQCRICM | HIV-A′ A |
| | RNFSRSDHLTTHIRTHTGEKPFACDICGRKFADYSVRKRHTK | |
| | IHTGGSGGSGERPYACPVESCDRRFSRSDELTRHIRIHTGQK | |
| | PFQCRICMRNFSRSDNLSTHIRTHTGEKPFACDICGRKFARR | |
| | DHRTTHTKIHL | |
| 47 | MAERPYACPVESCDRRFSDSAHLTRHIRIHTGQKPFQCRICM | HIV-BA |
| | RNFSRSDHLSTHIRTHTGEKPFACDICGRKFADSANRTKHTK | |
| | IHLRQKDGGSGGSGGSGGSGGSGGSERPYACPVESCDRRFSR | |
| | SDELTRHIRIHTGQKPFQCRICMRNFSRSDNLSTHIRTHTGE | |
| | KPFACDICGRKFARRDHRTTHTKIH | |
| 48 | MAERPYACPVESCDRRFSDSAHLTRHIRIHTGQKPFQCRICM | HIV-BA′ |
| | RNFSRSDHLSTHIRTHTGEKPFACDICGRKFADSANRTKHTK | |
| | IHTGGSGERPYACPVESCDRRFSRSDVLTRHIRIHTGQKPFQ | |
| | CRICMRNFSRSDHLTTHIRTHTGEKPFACDICGRKFADYSVR | |
| | KRHTKIH | |
| 49 | MAERPYACPVESCDRRFSRSDVLTRHIRIHTGQKPFQCRICM | HIV-A′ A-KOK |
| | RNFSRSDHLTTHIRTHTGEKPFACDICGRKFADYSVRKRHTK | |
| | IHTGGSGGSGERPYACPVESCDRRFSRSDELTRHIRIHTGQK | |
| | PFQCRICMRNFSRSDNLSTHIRTHTGEKPFACDICGRKFARR | |
| | DHRTTHTKIHLRQKDAARNSGPKKKRKVDGGGALSPQHSAVT | |
| | QGSIIKNKEGMDAKSLTAWSRTLVTFKDVFVDFTREEWLLLD | |
| | TAQQIVYRNVMLENYKNLVSLGYQLTKPDVILRLEKGEEPWL | |
| | VEREIHQETHPDSETAFEIKSSVEQKLISEEDL | |
| 50 | MAERPYACPVESCDRRFSDSAHLTRHIRIHTGQKPFQCRICM | HIV-BA-KOX |
| | RNFSRSDHLSTHIRTHTGEKPFACDICGRKFADSANRTKHTK | |
| | IHLRQKDGGSGGSGGSGGSGGSGGSERPYACPVESCDRRFSR | |
| | SDELTRHIRIHTGQKPFQCRICMRNFSRSDNLSTHIRTHTGE | |
| | KPFACDICGRKFARRDHRTTHTKIHLRQKDAARNSGPKKKRK | |
| | VDGGGALSPQHSAVTQGSIIKNKEGMDAKSLTAWSRTLVTFK | |
| | DVFVDFTREEWKLLDTAQQIVYRNVMLENYKNLVSLGYQLTK | |
| | PDVILRLEKGEEPWLVEREIHQETHPDSETAFEIKSSVEQKL | |
| | ISEEDL | |
| 51 | MAERPYACPVESCDRRFSDSAHLTRHIRIHTGQKPFQCRICM | HIV-BA′ -KOX |
| | RNFSRSDHLSTHIRTHTGEKPFACDICGRKFADSANRTKHTK | |
| | IHTGGSGERPYACPVESCDRRFSRSDVLTRHIRIHTGQKPFQ | |
| | CRICMRNFSRSDHLTTHIRTHTGEKPFACDICGRKFADYSVR | |
| | KRHTKIHLRQKDAARNSGPKKKRKVDGGGALSPQHSAVTQGS | |
| | IIKNKEGMDAKSLTAWSRTLVTFKDVFVDFTREEWKLLDTAQ | |
| | QIVYRNVMLENYKNLVSLGYQLTKPDVILRLEKGEEPWLVER | |
| | EIHQETHPDSETAFEIKSSVEQKLISEEDL | |

On pages 40 and 41, please replace the paragraph from line 8 on page 40 to page 41 with the following amended paragraph:

In a preferred embodiment of the invention, a nucleic acid binding polypeptide capable of binding a herpes virus nucleotide sequence comprises one or more of the following sequences:

| | | |
|---|---|---|
| SEQ ID NO: | Sequence | Name |

| | | |
|---|---|---|
| 52 | X₀₋₂ C X₁₋₅ C X₂₋₇ R S D E L T R H X₃₋₆ ^{H}/_{C} | 4/3 F1 |
| 53 | X₀₋₂ C X₁₋₅ C X₂₋₇ R S D H L S T H X₃₋₆ ^{H}/_{C} | 4/3 F2 |
| 54 | X₀₋₂ C X₁₋₅ C X₂₋₇ T N S N R I K H X₃₋₆ ^{H}/_{C} | 4/3 F3 |
| 55 | X₀₋₂ C X₁₋₅ C X₂₋₇ R S D E L T R H X₃₋₆ ^{H}/_{C} | 4A F1 |
| 56 | X₀₋₂ C X₁₋₅ C X₂₋₇ R S D H L S E H X₃₋₆ ^{H}/_{C} | 4A F2 |
| 57 | X₀₋₂ C X₁₋₅ C X₂₋₇ T N N N R K K H X₃₋₆ ^{H}/_{C} | 4A F3 |
| 58 | X₀₋₂ C X₁₋₅ C X₂₋₇ T R T N L T R H X₃₋₆ ^{H}/_{C} | 7N F1 |
| 59 | X₀₋₂ C X₁₋₅ C X₂₋₇ Q D A H L S T H X₃₋₆ ^{H}/_{C} | 7N F2 |
| 60 | X₀₋₂ C X₁₋₅ C X₂₋₇ Q S A N R K T H X₃₋₆ ^{H}/_{C} | 7N F3 |
| 61 | X₀₋₂ C X₁₋₅ C X₂₋₇ R S D E L T R H X₃₋₆ ^{H}/_{C} | 4/3 |
| | -linker-X₀₋₂ C X₁₋₅ C X₂₋₇ R S D H L S T | |
| | H X₃₋₆ ^{H}/_{C} -linker-X₀₋₂ C X₁₋₅ C X₂₋₇ T N | |
| | S N R I K H X₃₋₆ ^{H}/_{C} | |
| 62 | X₀₋₂ C X₁₋₅ C X₂₋₇ R S D E L T R H X₃₋₆ ^{H}/_{C} | 4A |
| | -linker-X₀₋₂ C X₁₋₅ C X₂₋₇ R S D H L S E | |
| | H X₃₋₆ ^{H}/_{C}-linker-X₀₋₂ C X₁₋₅ C X₂₋₇ T N | |
| | N N R K K H X₃₋₆ ^{H}/_{C} | |
| 63 | X₀₋₂ C X₁₋₅ C X₂₋₇ T R T N L T R H X₃₋₆ ^{H}/_{C} | 7N |
| | -linker-X₀₋₂ C X₁₋₅ C X₂₋₇ Q D A H L S T | |
| | H X₃₋₆ ^{H}/_{C}-linker-X₀₋₂ C X₁₋₅ C X₂₋₇ Q S | |
| | A N R K T H X₃₋₆ ^{H}/_{C} | |
| 64 | MAEERPYACPVESCDRRFSRSDELTRHIRIHTGQKPFQ | 4/3 |
| | CRICMRNFSRSDHLSTHIRTHTGEKPFACDICGRKFAT | |
| | NSNRIKHTKIHLRQKDAA | |
| 65 | MAEERPYACPVESCDRRFSRSDELTRHIRIHTGQKPFQ | 4A |
| | CRICMRNFSRSDHLSEHIRTHTGEKPFACDICGRKFAT | |
| | NNNRKKHTKIHLRQKDAA | |
| 66 | MAEERPYACPVESCDRRFSTRTNLTRHIRIHTGQKPFQ | 7N |
| | CRICMRNFSQDAHLSTHIRTHTGEKPFACDICGRKFAQ | |
| | SANRKTHTKIHLRQKDAA | |
| 67 | MAEERPYACPVESCDRRFSTRTNLTRHIRIHTGQKPFQ | 6F6 |
| | CRICMRNFSQDAHLSTHIRTHTGEKPFACDICGRKFAQ | |
| | SANRKTHTKIHLRQKDGERPYACPVESCDRRFSRSDEL | |
| | TRHIRIHTGQKPFQCRICMRNFSRSDHLSTHIRTHTGE | |
| | KPFACDICGRKFATNSNRIKHTKIHLRQKDAARNSTTL | |
| | D | |
| 68 | MAEERPYACPVESCDRRFSTRTNLTRHIRIHTGQKPFQ | 6F6-KOX |
| | CRICMRNFSQDAHLSTHIRTHTGEKPFACDICGRKFAQ | |
| | SANRKTHTKIHLRQKDGERPYACPVESCDRRFSRSDEL | |
| | TRHIRIHTGQKPFQCRICMRNFSRSDHLSTHIRTHTGE | |
| | KPFACDICGRKFATNSNRIKHTKIHLRQKDAARNSGPK | |
| | KRKVDGGGALSPQHSAVTQGSIIKNKEGMDAKSLTAWS | |
| | RTLVTFKDVFVDFTREEWKLLDTAQQIVYRNVMLENYK | |
| | NLVSLGYQLTKPDVILRLEKGEEPWLVEREIHQETHPD | |
| | SETAFEIKSSVEQKLISEDL | |

On pages 60 and 61, please replace the paragraph from line 25 on page 60 to line 14 on page 61 with the following amended paragraph:

The transcription factor binding site may be a binding site for a known transcription factor. The transcription factor may be an animal, preferably vertebrate, or plant transcription factor. Such transcription factors, and their putative or determined binding sites, including any consensus motifs, are known in the art, and may be found in (for example), the “Transcription Factor Database”, at http://www.hsc.virginia.edu/achs/molbio/databases/tfd_dat.html. Reference is also made to Nucleic Acids Res 21, 3117-8 (1993), Gene Transcription: A Practical Approach, 321-45 (1993) and Nucleic Acids Res 24, 238-41 (1996). A list of transcription factors, together with their binding sites, is contained in the file “tfsites.dat”, is a composite of the datasets TFD (release 7.5) SITES dataset file, March 1996 and Transfac (release 2.5) SITES dataset selected entries, January 1996. The file “tfsites.dat” may be obtained using the GCG command “FETCH tfsites.dat”. Any of these binding sites may be targeted according to the invention. Preferred transcription factors include those comprising homeodomains. Specific transcription factors and sites include those for NF-kB (GGGAAATTCC) (SEQ ID NO: 69), Sp1 (consensus sequence G/T-GGGCGG-G/A-G/A-C/T) (SEQ ID NO: 70) Oct-1 (ATTTGCAT), p53, myC, myB, API etc.

On page 72, please replace the paragraph from line 7 to line 16 with the following amended paragraph:

The following mutagenic protocol is used. The gene coding for the three zinc fingers of the wild-type Zif268 DNA-binding domain is altered by mutagenic PCR with the following primers:

| | |
|---|---|
| SfiVal3 (introduces a valine at position +3 of F1) | |
| 5′ GCAACTGCGGCCCAGCCGCCATGGCAGAGGAACGCCCATATGCTTGCCCTGTCGAGTCCTGC | (SEQ ID NO: 71) |
| GATCGCCGCTTTTCTCGCTCGGATGTCCTTACCCG-3′ | |
| F1 Val +3 | |
| NotGCC (introduces mutations in F3 to allow it to bind “GCC”) | |
| 5′ GAGTCATTCTGCGGCCGCGTCCTTCTGTCTTAAATGGATTTTGGTATGCCTCTTGCGCDMGC | (SEQ ID NO: 72) |
| TGKRGTSGGCAAACTTCCTCCC-3′ | |

On page 72, please replace the paragraph from line 18 to line 22 with the following amended paragraph:

After cloning the above PCR cassette into phage vector (by standard methods, as described previously) three rounds of selection are carried out (under standard selection conditions described herein) against a DNA target site containing the sequence: 5′-GCC TGG GCG G-3′ (SEQ ID NO: 73). The resulting Clone HIV-A′ (as shown in Table 1) binds its target sequence with a Kd of 5 nM, as measured by phage ELISA.

On page 73, please replace the paragraph from line 2 to line 5 with the following amended paragraph:

Using the above protocol, eight DNA-binding domains are produced (Table 1, Clones HIV-A to HIV-G and HIV-A′ (also known as Clone HIV-H; binds 5′-GCC TGG G(T/C)G-3′ (SEQ ID NO: 73)).

| | |
|---|---|
| DNA target | Zinc finger |
| sequence (a) | sequence (b) |

| | | | F1 | F2 | F3 | | F1 | F2 | F3 | |
|---|---|---|---|---|---|---|---|---|---|---|
| CLONE | SEQ ID NO | 3′-H | IJK | LMN | QPQ-5′ | SEQ ID NO | −1123456 | −1123456 | −1123456 | Kd/nM (c) |
| HIV-A | 74 | T | GCG | GAG | GGA | 81 | RSDELTR | RSDNLST | RRDHRTT | 1.2 ± 0.2 |
| HIV-A′ | 73 | G | GCG | GGT | CCG | 82 | RSDVLTR | TSDHLTT | DYSVRKR | 4.9 ± 0.4 |
| HIV-B | 75 | G | ACG | GGT | CAG | 83 | DSAHLTR | RSDHLST | DSANRTK | 1.0 ± 0.1 |
| HIV-C | 76 | T | ACG | TCG | TAG | 84 | ASADLTR | NRSDLSR | TSSNRKK | 13.7 ± 3.6 |
| HIV-D | 77 | T | TCG | TCG | ACG | 85 | HSSDLTR | QSSDLSK | QNATRKR | 4.0 ± 0.6 |
| HIV-E | 78 | T | CCG | AGT | CTA | 86 | DSSSLTK | QSAHLST | DSSSRTK | 36.6 ± 15.0 |
| HIV-F | 79 | T | CTC | TCG | AGG | 87 | ASDDLTQ | RSSDLSR | QSAHRTK | 13.3 ± 4.8 |
| HIV-G | 80 | G | GAT | CAA | TCG | 88 | RSDALTQ | DRANLST | ASSTRTK | 40.3 ± 14.6 |

On page 74, please replace the paragraph from line 24 to line 26 with the following amended paragraph:

The sequence of HIV-A (SEQ ID NO: 89) is

MAERPYACPVESCDRRFSRSDELTRHIRIHTGQKPFQCRICMRNFSRSDN
LSTHIRTHTGEKPFACDICGRKFARRDHRTTHTKIHLRQKD

On page 75, please replace the paragraphs from line 1 to line 6 with the following amended paragraphs:

The sequence of HIV-A′ (SEQ ID NO: 90) is

The sequence of HIV-A′ (SEQ IN NO: 90) is
MAERPYACPVESCDRRFSRSDVLTRHIRIHTGQKPFQCRICMRNFSRSDH
LTTHIRTHTGEKPFACDICGRKFADYSVRKRHTKIHLRQKD
The sequence of HIV-B (SEQ ID NO: 91) is
MAERPYACPVESCDRRFSDSAHLTRHIRIHTGQKPFQCRICMRNFSRSDH
LSTHIRTHTGEKPFACDICGRKFADSANRTKHTKIHLRQKD

On page 76, please replace the paragraphs from line 3 to line 22 with the following amended paragraphs:

HIV clones A′ and A are fused using the peptide linker sequence TGGSGGSGERP (SEQ ID NO: 92) to form HIV-A′A Clone HIV-A ′A has the following amino acid sequence (SEQ ID NO: 93)

MAERPYCPVESCDRRFSRSDVLTRHIRIHTGQKPFQCRICMRNFSRSDHL
TTHIRTHTGEKPFACDICGRKFADYSVRKRHTKIHTGGSGGSGERPYACP
VESCDRRFSRSDELTRHIRIHTGQKPFQCRICMRNFSRSDNLSTHIRTHT
GEKPFACDICGRKFARRDHRTTHTKIHLRQKD

HIV clones B and A are joined using the peptide linker sequence LRQKDGGSGGSGGSGGSGGSGGSERP (SEQ ID NO: 94) to form HIV-BA. Clone HIV-BA has the following amino acid sequence (SEQ ID NO: 95):

MAERPYACPVESCDRRFSDSAHLTRHIRIHTGQKPFQCRICMRNFSRSDH
LSTHIRTHTGEKPFACDICGRKFADSANRTKHTKIHLRQKDGGSGGSGGS
GGSGGSGGSERPYACPVESCDRRFSRSDELTRHIRIHTGQKPFQCRICMR
NFSRSDNLSTHIRTHTGEKPFACDICGRKFARRDHRTTHTKIHLRQKD

HIV clones B and A′ are fused using the peptide linker sequence TGGSGERP (SEQ ID NO: 96) to form HIV-BA′. Clone HIV-BA′ has the following amino acid sequence (SEQ ID NO: 97)

MAERPYACPVESCDRRFSDSAHLTRHIRIHTGQKPFQCRICMRNFSRSDH
LSTHIRTHTGEKPFACDICGRKFADSANRTKHTKIHTGGSGERPYACPVE
SCDRRFSRSDVLTRHIRIHTGQKPFQCRICMRNFSRSDHLTTHIRTHTGE
KPFACDICGRKFADYSVRKRHTKIHLRQKD

On page 77, please replace the paragraph from line 7 to line 15 with the following amended paragraph:

The KOX1 domain contains amino acids 1-97 from the human KOX1 protein (database accession code P21506) in addition to 23 amino acids which act as a linker. In addition, a 10 amino acid sequence from the c-myc protein (Evan et al., Mol. Cell. Biol. 5: 3610 (1985)) is introduced downstream of the KOX1 domain as a tag to facilitate expression studies of the fusion protein. The sequence of SV40-NLS-KOX1-c-myc repressor domain (NLS-KOX1-c-myc domain sequence) follows (SEQ ID NO: 98):

AARNSGPKKKRKVDGGGALSPQHSAVTQGSIIKNKEGMDAKSLTAWSRTL
VTFKDVFVDFTREEWKLLDTAQQIVYRNVMLENYKNLVSLGYQLTKPDVI
LRLEKGEEPWLVEREIHQETHPDSETAFEIKSSVEQKLISEEDL

On pages 77-81, please replace the paragraphs from line 21 on page 77 to line 27 on page 81 with the following amended paragraphs:

The nucleic acid sequence of HIV A-KOX is as follows (SEQ ID NO: 99):

ATGGCAGAGCGGCCGTATGCTTGCCCTGTCGAGTCCTGCGATCGCCGCTT
TTCTCGCTCGGATGAGCTTACCCGCCATATCCGCATCCACACAGGCCAGA
AGCCCTTCCAGTGTCGAATCTGCATGCGTAACTTCAGTCGTAGTGACAAC
CTGAGCACGCACATCCGCACCCACACAGGCGAGAAGCCTTTTGCCTGTGA
CATTTGTGGGAGGAAATTTGCCCGGAGGGACCACCGCACAACGCATACCA
AGATACACCTGCGCCAAAAAGATGCGGCCCGGAATTCCGGCCCAAAAAAG
AAGAGAAAGGTCGACGGCGGTGGTGCTTTGTCTCCTCAGCACTCTGCTGT
CACTCAAGGAAGTATCATCAAGAACAAGGAGGGCATGGATGCTAAGTCAC
TAACTGCCTGGTCCCGGACACTGGTGACCTTCAAGGATGTATTTGTGGAC
TTCACCAGGGAGGAGTGGAAGCTGCTGGACACTGCTCAGCAGATCGTGTA
CAGAAATGTGATGCTGGAGAACTATAAGAACCTGGTTTCCTTGGGTTATC
AGCTTACTAAGCCAGATGTGATCCTCCGGTTGGAGAAGGGAGAAGAGCCC
TGGCTGGTGGAGAGAGAAATTCACCAAGAGACCCATCCTGATTCAGAGAC
TGCATTTGAAATCAAATCATCAGTTGAACAAAAACTTATTTCTGAAGAAG
ATCTGTAA

The amino acid sequence of HIV A-KOX is as follows (SEQ ID NO: 100):

MAERPYACPVESCDRRFSRSDELTRHIRIHTGQKPFQCRICMRNFSRSDN
LSTHIRTHTGEKPFACDICGRKFARRDHRTTHTKIHLRQKDAARNSGPKK
KRKVDGGGALSPQHSAVTQGSIIKNKEGMDAKSLTAWSRTLVTFKDVFVD
FTREEWKLLDTAQQIVYRNVMLENYKNLVSLGYQLTKPDVILRLEKGEEP
WLVEREIHQETHPDSETAFEIKSSVEQKLISEEDL.

The nucleic acid sequence of HIV A′-KOX is as follows (SEQ ID NO: 101):

ATGGCAGAACGCCCGTATGCTTGCCCTGTCGAGTCCTGCGATCGCCGCTT
TTCTCGCTCGGATGTCCTTACCCGCCATATCCGCATCCACACAGGCCAGA
AGCCCTTCCAGTGTCGAATCTGCATGCGTAACTTCAGTCGTAGTGACCAC
CTTACCACCCACATCCGCACCCACACAGGCGAGAAGCCTTTTGCCTGTGA
CATTTGTGGGAGGAAGTTTGCCGACTACAGCGTACGCAAGAGGCATACCA
AAATCCATCTGCGCCAAAAAGATGCGGCCCGGAATTCCGGCCCAAAAAAG
AAGAGAAAGGTCGACGGCGGTGGTGCTTTGTCTCCTCAGCACTCTGCTGT
CACTCAAGGAAGTATCATCAAGAACAAGGAGGGCATGGATGCTAAGTCAC
TAACTGCCTGGTCCCGGACACTGGTGACCTTCAAGGATGTATTTGTGGAC
TTCACCAGGGAGGAGTGGAAGCTGCTGGACACTGCTCAGCAGATCGTGTA
CAGAAATGTGATGCTGGAGAACTATAAGAACCTGGTTTCCTTGGGTTATC
AGCTTACTAAGCCAGATGTGATCCTCCGGTTGGAGAAGGGAGAAGAGCCC
TGGCTGGTGGAGAGAGAAATTCACCAAGAGACCCATCCTGATTCAGAGAC
TGCATTTGAAATCAAATCATCAGTTGAACAAAAACTTATTTCTGAAGAAG
ATCTGTAA

The amino acid sequence of HIV A′-KOX is as follows (SEQ ID NO: 102):

MERPYACPVESCDRRFSRSDVLTRHIRIHTGQKPFQCRICMRNFSRSDHL
TTHIRTHTGEKPFACDICGRKFADYSVRKRHTKIHLRQKDAARNSGPKKK
RKVDGGGALSPQHSAVTQGSIIKNKEGMDAKSLTAWSRTLVTFKDVFVDF
TREEWKLLDTAQQIVYRNVMLENYKNLVSLGYQLTKPDVILRLEKGEEPW
LVEREIHQETHPDSETAFEIKSSVEQKLISEEDL.

The nucleic acid sequence of HIVB-KOX is as follows (SEQ ID NO: 103):

ATGGCGGAGAGGCCCTACGCATGCCCTGTCGAGTCCTGCGATCGCCGCTT
TTCTGACTCGGCCCACCTTACCCGGCATATCCGCATCCACACCGGTCAGA
AGCCCTTCCAGTGTCGAATCTGCATGCGTAACTTCAGTCGGAGCGACCAC
CTGAGCACCCACATCCGCACCCACACAGGCGAGAAGCCTTTTGCCTGTGA
CATTTGTGGGAGGAAATTTGCCGACAGCGCCAACCGCACAAAGCATACCA
AGATACACCTGCGCCAAAAAGATGCGGCCCGGAATTCCGGCCCAAAAAAG
AAGAGAAAGGTCGACGGCGGTGGTGCTTTGTCTCCTCAGCACTCTGCTGT
CACTCAAGGAAGTATCATCAAGAACAAGGAGGGCATGGATGCTAAGTCAC
TAACTGCCTGGTCCCGGACACTGGTGACCTTCAAGGATGTATTTGTGGAC
TTCACCAGGGAGGAGTGGAAGCTGCTGGACACTGCTCAGCAGATCGTGTA
CAGAAATGTGATGCTGGAGAACTATAAGAACCTGGTTTCCTTGGGTTATC
AGCTTACTAAGCCAGATGTGATCCTCCGGTTGGAGAAGGGAGAAGAGCCC
TGGCTGGTGGAGAGAGAAATTCACCAAGAGACCCATCCTGATTCAGAGAC
TGCATTTGAAATCAAATCATCAGTTGAACAAAAACTTATTTCTGAAGAAG
ATCTGTAA

The amino acid sequence of HIVB-KOX is as follows (SEQ ID NO: 104):

MERPYACPVESCDRRFSDSAHLTRHIRIHTGQKPFQCRICMRNFSRSDHL
STHIRTHTGEKPFACDICGRKFADSANRTKHTKIHLRQKDAARNSGPKKK
RKVDGGGALSPQHSAVTQGSIIKNKEGMDAKSLTAWSRTLVTFKDVFVDF
TREEWKLLDTAQQIVYRNVMLENYKNLVSLGYQLTKPDVILRLEKGEEPW
LVEREIHQETHPDSETAFEIKSSVEQKLISEEDL.

The nucleic acid sequence of HIV A′A-KOX is as follows (SEQ ID NO: 105):

ATGGCAGAACGCCCGTATGCTTGCCCTGTCGAGTCCTGCGATCGCCGCTT
TTCTCGCTCGGATGTCCTTACCCGCCATATCCGCATCCACACAGGCCAGA
AGCCCTTCCAGTGTCGAATCTGCATGCGTAACTTCAGTCGTAGTGACCAC
CTTACCACCCACATCCGCACCCACACAGGCGAGAAGCCTTTTGCCTGTGA
CATTTGTGGGAGGAAGTTTGCCGACTACAGCGTACGCAAGAGGCATACCA
AAATCCATACCGGCGGGAGCGGCGGGAGCGGCGAGCGGCCGTATGCTTGC
CCTGTCGAGTCCTGCGATCGCCGCTTTTCTCGCTCGGATGAGCTTACCCG
CCATATCCGCATCCACACAGGCCAGAAGCCCTTCCAGTGTCGAATCTGCA
TGCGTAACTTCAGTCGTAGTGACAACCTGAGCACGCACATCCGCACCCAC
ACAGGCGAGAAGCCTTTTGCCTGTGACATTTGTGGGAGGAAATTTGCCCG
GAGGGACCACCGCACAACGCATACCAAGATACACCTGCGCCAAAAAGATG
CGGCCCGGAATTCCGGCCCAAAAAAGAAGAGAAAGGTCGACGGCGGTGGT
GCTTTGTCTCCTCAGCACTCTGCTGTCACTCAAGGAAGTATCATCAAGAA
CAAGGAGGGCATGGATGCTAAGTCACTAACTGCCTGGTCCCGGACACTGG
TGACCTTCAAGGATGTATTTGTGGACTTCACCAGGGAGGAGTGGAAGCTG
CTGGACACTGCTCAGCAGATCGTGTACAGAAATGTGATGCTGGAGAACTA
TAAGAACCTGGTTTCCTTGGGTTATCAGCTTACTAAGCCAGATGTGATCC
TCCGGTTGGAGAAGGGAGAAGAGCCCTGGCTGGTGGAGAGAGAAATTCAC
CAAGAGACCCATCCTGATTCAGAGACTGCATTTGAAATCAAATCATCAGT
TGAACAAAAACTTATTTCTGAAGAAGATCTGTAA

The amino acid sequence of HIVA′A-KOX is as follows (SEQ ID NO: 106):

MERPYACPVESCDRRFSRSDVLTRHIRIHTGQKPFQCRICMRNFSRSDHL
TTHIRTHTGEKPFACDICGRKFADYSVRKRHTKIHTGGSGGSGERPYACP
VESCDRRFSRSDELTRHIRIHTGQKPFQCRICMRNFSRSDNLSTHIRTHT
GEKPFACDICGRKFARRDHRTTHTKIHLRQKDAARNSGPKKKRKVDGGGA
LSPQHSAVTQGSIIKNKEGMDAKSLTAWSRTLVTFKDVFVDFTREEWKLL
DTAQQIVYRNVMLENYKNLVSLGYQLTKPDVILRLEKGEEPWLVEREIHQ
ETHPDSETAFEIKSSVEQKLISEEDL . . .

The nucleic acid sequence of HIVBA-KOX is as follows (SEQ ID NO: 107):

ATGGCGGAGAGGCCCTACGCATGCCCTGTCGAGTCCTGCGATCGCCGCTT
TTCTGACTCGGCCCACCTTACCCGGCATATCCGCATCCACACCGGTCAGA
AGCCCTTCCAGTGTCGAATCTGCATGCGTAACTTCAGTCGGAGCGACCAC
CTGAGCACCCACATCCGCACCCACACAGGCGAGAAGCCTTTTGCCTGTGA
CATTTGTGGGAGGAAATTTGCCGACAGCGCCAACCGCACAAAGCATACCA
AGATACACCTGCGCCAAAAAGATGGGGGCAGCGGCGGGTCCGGGGGGAGC
GGCGGCTCCGGGGGCAGCGGCGGGTCCGAGCGGCCGTATGCTTGCCCTGT
CGAGTCCTGCGATCGCCGCTTTTCTCGCTCGGATGAGCTTACCCGCCATA
TCCGCATCCACACAGGCCAGAAGCCCTTCCAGTGTCGAATCTGCATGCGT
AACTTCAGTCGTAGTGACAACCTGAGCACGCACATCCGCACCCACACAGG
CGAGAAGCCTTTTGCCTGTGACATTTGTGGGAGGAAATTTGCCCGGAGGG
ACCACCGCACAACGCATACCAAGATACACCTGCGCCAAAATGAGCACGCA
CATCCGCACCCACACAGGCGAGAAGCCTTTTGCCTGTGACATTTGTGGGA
GGAAATTTGCCCGGAGGGACCACCGCACAACGCATACCAAGATACACCTG
CGCCAAAAAGATGCGGCCCGGAATTCCGGCCCAAAAAAGAAGAGAAAGGT
CGACGGCGGTGGTGCTTTGTCTCCTCAGCACTCTGCTGTCACTCAAGGAA
GTATCATCAAGAACAAGGAGGGCATGGATGCTAAGTCACTAACTGCCTGG
TCCCGGACACTGGTGACCTTCAAGGATGTATTTGTGGACTTCACCAGGGA
GGAGTGGAAGCTGCTGGACACTGCTCAGCAGATCGTGTACAGAAATGTGA
TGCTGGAGAACTATAAGAACCTGGTTTCCTTGGGTTATCAGCTTACTAAG
CCAGATGTGATCCTCCGGTTGGAGAAGGGAGAAGAGCCCTGGCTGGTGGA
GAGAGAAATTCACCAAGAGACCCATCCTGATTCAGAGACTGCATTTGAAA
TCAAATCATCAGTTGAACAAAAACTTATTTCTGAAGAAGATCTGTAA

The amino acid sequence of HIVBA-KOX is as follows (SEQ ID NO: 108):

MAERPYACPVESCDRRFSDSAHLTRHIRIHTGQKPFQCRICMRNFSRSDH
LSTHIRTHTGEKPFACDICGRKFADSANRTKHTKIHLRQKDGGSGGSGGS
GGSGGSGGSERPYACPVESCDRRFSRSDELTRHIRIHTGQKPFQCRICMR
NFSRSDNLSTHIRTHTGEKPFACDICGRKFARRDHRTTHTKIHLRQKDAA
RNSGPKKKRKVDGGGALSPQHSAVTQGSIIKNKEGMDAKSLTAWSRTLVT
FKDVFVDFTREEWKLLDTAQQIVYRNVMLENYKNLVSLGYQLTKPDVILR
LEKGEEPWLVEREIHQETHPDSETAFEIKSSVEQKLISEEDL.

The nucleic acid sequence of HIVBA′-KOX is as follows (SEQ ID NO: 109):

ATGGCGGAGAGGCCCTACGCATGCCCTGTCGAGTCCTGCGATCGCCGCTT
TTCTGACTCGGCCCACCTTACCCGGCATATCCGCATCCACACCGGTCAGA
AGCCCTTCCAGTGTCGAATCTGCATGCGTAACTTCAGTCGGAGCGACCAC
CTGAGCACCCACATCCGCACCCACACAGGCGAGAAGCCTTTTGCCTGTGA
CATTTGTGGGAGGAAATTTGCCGACAGCGCCAACCGCACAAAGCATACCA
AGATACACACCGGCGGGAGCGGCGAGCGGCCGTATGCTTGCCCTGTCGAG
TCCTGCGATCGCCGCTTTTCTCGCTCGGATGTCCTTACCCGCCATATCCG
CATCCACACAGGCCAGAAGCCCTTCCAGTGTCGAATCTGCATGCGTAACT
TCAGTCGTAGTGACCACCTTACCACCCACATCCGCACCCACACAGGCGAG
AAGCCTTTTGCCTGTGACATTTGTGGGAGGAAGTTTGCCGACTACAGCGT
GCGCAAGAGGCATACCAAAATCCATTTAAGACAGAAGGACGCGGCCCGGA
ATTCCGGCCCAAAAAAGAAGAGAAAGGTCGACGGCGGTGGTGCTTTGTCT
CCTCAGCACTCTGCTGTCACTCAAGGAAGTATCATCAAGAACAAGGAGGG
CATGGATGCTAAGTCACTAACTGCCTGGTCCCGGACACTGGTGACCTTCA
AGGATGTATTTGTGGACTTCACCAGGGAGGAGTGGAAGCTGCTGGACACT
GCTCAGCAGATCGTGTACAGAAATGTGATGCTGGAGAACTATAAGAACCT
GGTTTCCTTGGGTTATCAGCTTACTAAGCCAGATGTGATCCTCCGGTTGG
AGAAGGGAGAAGAGCCCTGGCTGGTGGAGAGAGAAATTCACCAAGAGACC
CATCCTGATTCAGAGACTGCATTTGAAATCAAATCATCAGTTGAACAAAA
CTTATTTCTGAAGAAGATCTGTAA

The amino acid sequence of HIVBA′-KOX is as follows (SEQ ID NO: 110):

MAERPYACPVESCDRRFSDSAHLTRHIRIHTGQKPFQCRICMRNFSRSDH
LSTHIRTHTGEKPFACDICGRKFADSANRTKHTKIHTGGSGERPYACPVE
SCDRRFSRSDVLTRHIRIHTGQKPFQCRICMRNFSRSDHLTTHIRTHTGE
KPFACDICGRKFADYSVRKRHTKIHLRQKDAARNSGPKKKRKVDGGGALS
PQHSAVTQGSIIKNKEGMDAKSLTAWSRTLVTFKDVFVDFTREEWKLLDT
AQQIVYRNVMLENYKNLVSLGYQLTKPDVILRLEKGEEPWLVEREIHQET
HPDSETAFEIKSSVEQKLISEEDL.

On pages 96 and 97, please replace the paragraph from line 22 on page 96 to line 1 on page 97 with the following amended paragraph:

Two 9 bp sequences (named t, t2 and t4 shown below), spanning the transactivation complex binding region (including TAATGARAT—underlined on IE175k promoter sequence (SEQ ID NO: 111) shown below), are chosen as targets for zinc finger factors.

−270

(SEQ ID NO: 111)

| | |
|---|---|
| GATCGGGCGGTAATGAGATGCCATG | HSV IE175k |
| TAATGAGAT | t2 |
| GATCGGGCGG | t4 |

On pages 97 and 98, please replace the paragraphs from line 9 on page 97 to line 2 on page 98 with the following amended paragraphs:

The nucleic acid sequence of Clone 4/3 is as follows (SEQ ID NO: 112):

ATGGCAGAGGAACgccatatgctTGCCCTGTCGAGTCCTGCGATCGCCGC
TTTTCTCGCTCGGATGAGCTTACCCGCCATATCCGCATCCACACAGGCCA
GAAGCCCTTCCAGTGTCGAATCTGCATGCGTAACTTCAGTCGTAGTGACC
ACCtgaGCACGCACATCCGCACCCACACAGGCGAGAAGCCTTTTGCCTGT
GACATTTGTGGGAGGAaattTGCCACCAACAGCAACCGCATAAAGCATAC
CAAGATACACCTGCGCCAAAAAGATGCGGCC

The amino acid sequence of Clone 4/3 is as follows (SEQ ID NO: 113):

MAEERPYACPVESCDRRFSRSDELTRHIRIHTGQKPFQCRICMRNFSRSD
HLSTHIRTHTGEKPFACDICGRKFATNSNRIKHTKIHLRQKDAA

The nucleic acid sequence of Clone 4A is as follows (SEQ ID NO: 114):

ATGGCAGAGGAACgcccatatgctTGCCCTGTCGAGTCCTGCGATCGCCG
CTTTTCTCGCTCGGATGAGCTTACCCGCCATATCCGCATCCACACAGGCC
AGAAGCCCTTCCAGTGTCGAATCTGCATGCGTAACTTCAGTCGTAGTGAC
CACCtgaGCGAGCACATCCGCACCCACACAGGCGAGAAGCCTTTTGCCTG
TGACATTTGTGGGAGGAaattTGCCACCAACAACAACGCAAAAAGCATAC
CAAGATACACCTGCGCCAAAAAGATGCGGCC

The nucleic amino acid sequence of Clone 4A is as follows (SEQ ID NO: 115): MAEERPYACPVESCDRRFSRSDELTRHIRIHTGQKPFQCRICMRNFSRSDHLSEHIRTHTGEKPFACDICGRKFATNNNRKKHTKIHLRQKDAA

On pages 98-100, please replace the paragraphs from line 15 on page 98 to line 11 on page 100 with the following amended paragraphs:

The nucleotide sequence of Clone 7N is as follows (SEQ ID NO: 116):

ATGGCAGAGGAACgcccatatgctTGCCCTGTCGAGTCCTGCGATCGCCG
CTTTTCTACGCGAACTAACCTTACCCGCCCATATCCGCATCCACACAGGC
CAGAAGCCCTTCCAGTGTCGAATCTGCATGCGTAACTTCAGTCAGGACGC
ACACCtgaGCACGCACATCCGCACCCACACAGGCGAGAAGCCTTTTGCCT
GTGACATTTGTGGGAGGAaattTGCCCAGAGCGCCAACCGCAAAACGCAT
ACCAAGATACACCTGCGCCAAAAAGATGCGGCC

The amino acid sequence of Clone 7N is as follows (SEQ ID NO: 117):

MAEERPYACPVESCDRRFSTRTNLTRHIRIHTGQKPFQCRICMRNFSQDA
HLSTHIRTHTGEKPFACDICGRKFAQSANRKTHTKIHLRQKDAA

Furthermore, six finger constructs were produced from the three finger clones (for example, 6F6 is a finger protein comprising 7N and 4/3, which binds GATCGGGCG g TAATGAGAT (SEQ ID NO:111)).

The nucleic acid sequence of Clone 6F6 is as follows (SEQ ID NO: 118):

ATGGCAGAGGAACgcccatatgctTGCCCTGTCGAGTCCTGCGATCGCCG
CTTTTCTACGCGAACTAACCTTACCCGCCATATCCGCATCCACACAGGCC
AGAAGCCCTTCCAGTGTCGAATCTGCATGCGTAACTTCAGTCAGGACGCA
CACCtgaGCACGCACATCCGCACCCACACAGGCGAGAAGCCTTTTGCCTG
TGACATTTGTGGGAGGAaattTGCCCAGAGCGCCAACCGCAAAACGCATA
CCAAGATACACCTGCGCCAAAAAGATGGCGAACgcccatatgctTGCCCT
GTCGAGTCCTGCGATCGCCGCTTTTCTCGCTCGGATGAGCTTACCCGCCA
TATCCGCATCCACACAGGCCAGAAGCCCTTCCAGTGTCGAATCTGCATGC
GTAACTTCAGTCGTAGTGACCACCtgaGCACGCACATCCGCACCCACACA
GGCGAGAAGCCTTTTGCCTGTGACATTTGTGGGAGGAaattTGCCACCAA
CAGCAACCGCATAAAGCATACCAAGATACACCTGCGCCAAAAAGATGCGG
CCCGGAATTCCACCACACTGGACTAG

The amino acid sequence of Clone 6F6 is as follows (SEQ ID NO: 119):

MAEERPYACPVESCDRRFSTRTNLTRHIRIHTGQKPFQCRICMRNFSQDA
HLSTHIRTHTGEKPFACDICGRKFAQSANRKTHTKIHLRQKDGERPYACP
VESCDRRFSRSDELTRHTRIHTGQKPFQCRICMRNFSRSDHLSTHIRTHT
GEKPFACDICGRKFATNSNRIKHTKIHLRQKDAARNSTTLD

Clone 6F6 is also fused with the KRAB repression domain of KOX to produce 6F6-KOX.

The nucleic acid sequence of 6F6-KOX is as follows (SEQ ID NO: 120):

ATGGCAGAGGAACgcccatatgctTGCCCTGTCGAGTCCTGCGATCGCCG
CTTTTCTACGCGAACTAACCTTACCCGCCATATCCGCATCCACACAGGCC
AGAAGCCCTTCCAGTGTCGAATCTGCATGCGTAACTTCAGTCAGGACGCA
CACCtgaGCACGCACATCCGCACCCACACAGGCGAGAAGCCTTTTGCCTG
TGACATTTGTGGGAGGAaattTGCCCAGAGCGCCAACCGCAAAACGCATA
CCAAGATACACCTGCGCCAAAAAGATGGCGAACgcccatatgctTGCCCT
GTCGAGTCCTGCGATCGCCGCTTTTCTCGCTCGGATGAGCTTACCCGCCA
TATCCGCATCCACACAGGCCAGAAGCCCTTCCAGTGTCGAATCTGCATGC
GTAACTTCAGTCGTAGTGACCACCtgaGCACGCACATCCGCACCCACACA
GGCGAGAAGCCTTTTGCCTGTGACATTTGTGGGAGGAaattTGCCACCAA
CAGCAACCGCATAAAGCATACCAAGATACACCTGCGCCAAAAAGATGCGG
CCcggaattccggcccaaaaaagagaaaggtcgacggcggtggtgctttg
tctcctcagcactctgctgtcactcaaggaagtatcatcaagaacaagga
gggcatggatgctaagtcactaactgcctggtcccggacactggtgacct
tcaaggatgtatttgtggacttcaccagggaggagtggaagctgctggac
actgctcagcagatcgtgtacagaaatgtgatgctggagaactataagaa
cctggtttccttgggttatcagcttactaagccagatgtgatcctccggt
tggagaagggagaagagccctggctggtggagagagaaattcaccaagag
acccatcctgattcagagactgcatttgaaatcaaatcatcagttgaaca
aaaacttatttctgaagatctgtaa

The amino acid sequence of 6F6-KOX is as follows (SEQ ID NO: 121):

MAEERPYACPVESCDRRFSTRTNLTRHIRIHTGQKPFQCRICMRNFSQDA
HLSTHTRTHTGEKPFACDICGRKFAQSANRTKTHTKIHLRQKDGERPYAC
PVESCDRRFSRSDELTRHIRIHTGQKPFQCRICMRNFSRSDHLSTHIRTH
TGEKPFACDICGRKFATNSNRIKHTKIHLRQKDAARNSGPKKRKVDGGGA
LSPQHSAVTQGSIIKNKEGMDAKSLTAWSRTLVTFKDVFVDFTREEWKLL
DTAQQIVYRNVMLENYKNLVSLGYQLTKPDVILRLEKGEEPWLVEREIHQ
ETHPDSETAFEIKSSVEQKLISEDL*

On page 100, please replace the paragraph from line 14 to line 25 with the following amended paragraph:

Primers Used for PCR Cloning

| | |
|---|---|
| 4AFOR: | |
| CTG CTC TAG AGC GCC GCC | (SEQ ID NO: 122) |
| ATG GCA GAG GAA CGC; | |
| HIV13Rev: | |
| TCC GGG ATC CCG CGG AAT | (SEQ ID NO: 123) |
| TCC GGG CCG CAT CTT TTT | |
| GGC GCA GGT G; | |
| HIV13For: | |
| CTC TAG AGC GCC GCC ATG | (SEQ ID NO: 124) |
| GCG GAA GAG AGG CCC; | |
| NCFUS2: | |
| GAA ACG CCC ATA TGC TTG | (SEQ ID NO: 125) |
| CCC TGT C; | |
| RevlinGLY: | |
| CAG GGC AAG CAT ATG GGC | (SEQ ID NO: 126) |
| GTT C GCC ATC TTT TTG | |
| GCG CAG GTG TAT CTT GG; | |
| FOR2: | |
| GA CAG AAG GAC GCG GCC | (SEQ ID NO: 127) |
| ACG CGT CCA AAA AAG AAG | |
| AGA AAG GTC; | |
| REV2: | |
| CGC GGA TCC TTA CAG ATC | (SEQ ID NO: 128) |
| TTC TTC AGA AAT AAG TTT | |
| TTG TTC AAC TGA TGA TTT | |
| GAT TTC AAA TGC; | |
| 6F6HIND FOR: | |
| CTA CGT AAG CTT GCG CCG | (SEQ ID NO: 129) |
| CCA TGG CAG AGG AAC G; | |
| KOX/VP16REV: | |
| GCT CGG ATC CTT ACA GAT | (SEQ ID NO: 130) |
| CTT CTT CAG A | |

On page 104, please replace the paragraph from line 7 to line 12 with the following amended paragraph:

The sequences of molecular probes used for gel retardation assays are as follow:

(SEQ ID NO: 131)

| | |
|---|---|
| T24: | CCG CCG GAT CGG GCG G TAA TGA GAT GCC ATG |

(SEQ ID NO: 132)

| | |
|---|---|
| H2B: | ATA GAA TCG CTT ATG C AAA TAA GGT GAA GA |

(SEQ ID NO: 133)

| | |
|---|---|
| 68K: | CTT CCC GGT TCG GCG G TAA TGA GAT ACG AG |

(SEQ ID NO: 134)

| | |
|---|---|
| IE110: | TGG GTT CCG GGT ATG G TAA TGA GTT TCT TC |

On page 107, please replace the paragraphs from line 15 to line 22 with the following amended paragraphs:

### Example 18

### Analysis 6-Finger Protein Binding T4+T2 (GATCGGGCGGTAATGAGAT) (SEQ ID NO:111)

In an attempt to create a strong binder (capable of in vivo HSV inhibition via binding to the complete t4+t2 site), the 4/3 and 7N 3-finger proteins are fused using the amino acid sequence QKDGERP (SEQ ID NO: 135) as a linker to form a 6-finger protein (6F6). The resulting 6-finger protein (6F6) is capable of binding one of the two TAATGARAT sequences (+adjacent region) present in the IE175k promoter (position −230 in respect to the start of transcription).

1. A polypeptide capable of binding to a nucleic acid comprising a viral nucleotide sequence.

2. A polypeptide according to claim 1, in which the viral nucleotide sequence comprises a viral promoter sequence.

3. A polypeptide according to claim 1 or 2, in which the viral promoter sequence comprises a Human Immunodeficiency Virus (HIV) promoter sequence.

4. A polypeptide according to any preceding claim, in which the polypeptide comprises a zinc finger motif having a general primary structure:

| (A′) X₀₋₂ C X₁₋₅ C X₂₋₇ | X X X X X X X H X₃₋₆ ^{H}/_{C} |
|---|---|
| | −1 1 2 3 4 5 6 7 |

where X is any amino acid, and the numbers in subscript indicate the possible numbers of residues represented by X in which the amino acids at positions −1, 1, 2, 3, 4, 5 and 6 are selected from the group consisting of: RSDELTR, RSDNLST, RRDHRTT, RSDVLTR, RSDHLTT, DYSVRKR, DSAHLTR, RSDHLST, DSANRTK, ASADLTR, NRSDLSR, TSSNRKK, HSSDLTR, QSSDLSK, QNATRKR, DSSSLTK, QSAHLST, DSSSRTK, ASDDLTQ, RSSDLSR, QSAHRTK, RSDALIQ, DRANLST, ASSTRTK.

5. A polypeptide according to claim 4, in which the polypeptide comprises three zinc finger motifs F1, F2 and F3, in which the amino acids at positions −1, 1, 2, 3, 4, 5 and 6 of F1, F2 and F3 are selected from the group consisting of:

| | | | |
|---|---|---|---|
| (a) | F1: RSDELTR, | F2: RSDNLST, | F3: RRDHRTT; |
| (b) | F1: RSDVLTR, | F2: RSDHLTT, | F3: DYSVRKR; |
| (c) | F1: DSAHLTR, | F2: RSDHLST, | F3: DSANRTK. |

6. A polypeptide according to claim 4 or 5, in which the polypeptide comprises six zinc finger motifs F1 to F6, in which the amino acids at positions −1, 1, 2, 3, 4, 5 and 6 of F1, F2, F3, F4, F5 and F6 are selected from the group consisting of:

| | | | |
|---|---|---|---|
| (a) | F1: RSDVLTR, | F2: RSDHLTT, | F3: DYSVRKR, |
| | F4: RSDELTR, | F5: RSDNLST, | F6: RRDHRTT; |
| (b) | F1: DSAHLTR, | F2: RSDHLST, | F3: DSANRTK, |
| | F4: RSDELTR, | F5: RSDNLST, | F6: RRDHRTT; |
| (c) | F1: DSAHLTR, | F2: RSDHLST, | F3: DSANRTK, |
| | F4: RSDVLTR, | F5: RSDHLTT, | F6: DYSVRKR. |

7. A polypeptide according to any preceding claim, in which the polypeptide is selected from the group consisting of: HIV-A, HIV-A′, HIV-B, HIV-C, HIV-D, HIV-E, HIV-F, HIV-G, HIV-A′A, HIV-BA and HIV-BA′.

8. A polypeptide according to claim 1 or 2, in which the viral promoter sequence comprises a herpesvirus promoter sequence.

9. A polypeptide according to any of claims 1, 2 or 8, in which the polypeptide comprises a zinc finger motif having a general primary structure:

| (A′) X₀₋₂ C X₁₋₅ C X₂₋₇ | X X X X X X X H X₃₋₆ ^{H}/_{C} |
|---|---|
| | −1 1 2 3 4 5 6 7 |

where X is any amino acid, and the numbers in subscript indicate the possible numbers of residues represented by X, in which the amino acids at positions −1, 1, 2, 3, 4, 5 and 6 are selected from the group consisting of: RSDELTR, RSDHLST, TNSNRIK, RSDELTR, RSDHLST, TNSNRIK, TRTNLTR, QDAHLST and QSANRKT.

10. A polypeptide according to claim 9, in which the polypeptide comprises three zinc finger motifs F1, F2 and F3, in which the amino acids at positions −1, 1, 2, 3, 4, 5 and 6 of F1, F2 and F3 are selected from the group consisting of:

| | | | |
|---|---|---|---|
| (a) | F1: RSDELTR, | F2: RSDHLST, | F3: TNSNRIK |
| (b) | E1: RSDELTR, | F2: RSDHLST, | F3: TNSNRIK |
| (c) | F1: TRTNLTR, | P2: QDAHLST, | F3: QSANRKT. |

11. A polypeptide according to claim 9 or 10, in which the polypeptide comprises six zinc finger motifs F1 to F6, in which the amino acids at positions −1, 1, 2, 3, 4, 5 and 6 of F1 comprise TRTNLTR, of F2 comprise QDAHLST, of F3 comprise QSANRKT, of F4 comprise RSDELTR, of F5 comprise RSDHLST, and of F6 comprise TNSNRIK.

12. A polypeptide according to any preceding claim, in which the polypeptide is selected from the group consisting of: 4/3, 4A, and 7N.

13. A polypeptide according to any preceding claim, which further comprises a transcriptional effector domain.

14. A polypeptide according to claim 13, in which the transcriptional effector domain is a repressor domain selected from the group comprising a KRAB-A domain, an engrailed domain and a snag domain.

15. A polypeptide according to claim 13 or 14, which is selected from the group consisting of: HIV-A-KOX, HIV-A′-KOX, HIV-B-KOX HIV-A′A-KOX HIV-BA-KOX, HIV-BA′-KOX and 6F6-KOX.

16. A polypeptide according to any preceding claim, in which the polypeptide is capable of repressing transcription from a viral promoter.

17. A polypeptide according to any preceding claim selected by phage display.

18. A composition comprising a pharmaceutically effective amount of a polypeptide according to any preceding claim, together with a pharmaceutically acceptable excipient, diluent or carrier.

19. A nucleic acid molecule encoding a polypeptide according to any of claims 1 to 17.

20. An expression vector comprising a nucleic acid molecule according to claim 19.

21. A particle harbouring a polypeptide according to any of claims 1 to 17, a nucleic acid according to claim 19, or an expression vector according to claim 20.

22. A method of modulating transcription by targeting nucleic acid sequences that overlap with transcription factor binding sites by the use of engineered zinc finger molecules.

23. A method of modulating transcription of a nucleic acid molecule comprising contacting said nucleic acid molecule with a polypeptide according to any of claims 1 to 17.

24. A method according to claim 23, in which the polypeptide binds to a nucleic acid sequence comprising a transcription factor binding site or a variant or part thereof.

25. A method according to claim 23, in which the polypeptide binds to a nucleic acid sequence adjacent to a transcription factor binding site or a variant or part thereof.

26. A method according to claim 23, in which the polypeptide binds to more than one nucleic acid sequence, each nucleic acid sequence comprising or being adjacent to a transcription factor binding site or a variant or part thereof.

27. A method of modulating transcription of a nucleic acid molecule comprising contacting the nucleic acid molecule with two or more polypeptides according to any of claims 1 to 17.

28. A method of modulating transcription from a HIV promoter comprising contacting a nucleic acid comprising HIV promoter with a polypeptide according to any of claims 1 to 7 or 13 to 17 as dependent thereon.

29. A method of modulating transcription from a herpesvirus promoter comprising contacting a nucleic acid comprising the herpesvirus promoter with a polypeptide according to any of claims 1, 2, 8 to 12 or 13 to 17 as dependent thereon.

30. Use of a zinc finger polypeptide, or a nucleic acid encoding such a polypeptide, to modulate transcription of a viral nucleotide sequence.

31. A method of treating a disease in a patient caused by a virus, the method comprising administering a zinc finger polypeptide capable of binding to a viral nucleotide sequence, or a nucleic acid encoding such a polypeptide, to the patient.

32. A zinc finger polypeptide, or a nucleic acid encoding such a polypeptide, for use in a method of treatment of a disease caused by a virus.

33. Use of a zinc finger polypeptide, or a nucleic acid encoding such a polypeptide, in the preparation of a medicament for use in the treatment of a disease caused by a virus in a patient.

34. Use according to claim 30 or 33, a method according to claim 31, or a polypeptide or nucleic acid according to claim 32, in which the zinc finger polypeptide comprises a polypeptide according to any of claims 1 to 17.

35. A method of treating a disease in a patient, the method comprising introducing a nucleic acid sequence encoding a nucleic acid binding polypeptide into a cell of a patient, such that the nucleic acid sequence is capable of being propagated to daughter cells of the introduced cell.

36. A method according to claim 35, in which the nucleic acid is stably integrated into the cell.

37. A method according to claim 35 or 36, in which the nucleic acid sequence encodes a polypeptide according to any of claims 1 to 17.

38. A method of targeting a native viral nucleic acid sequence with a nucleic acid binding polypeptide, the method comprising: (a) providing a nucleic acid binding polypeptide; (b) providing a native viral nucleic acid sequence comprising one or more nucleotide sequences capable of being bound by the nucleic acid binding polypeptide; and (b) contacting the nucleic acid binding polypeptide with the native viral nucleic acid sequence.

39. A method according to claim 38, in which the native viral nucleic acid mediates the infection of a cell by a virus.

40. A method according to claim 37 or 38, in which the native viral nucleic acid sequence comprises a provirus or an virus integrated into the genome of a host cell.

41. A method of downregulating a viral function in a cell infected with the virus, the method comprising contacting the virus and/or the cell with a nucleic acid binding polypeptide capable of binding a nucleic acid sequence of the virus.

42. A method of modulating a viral function in a system comprising administering a polypeptide according to any preceding claim to said system.

43. A method according to claim 41 or 42, in which the viral function is selected from the group consisting of: viral titre, viral infectivity, viral replication, viral packaging, and viral transcription.


SEQUENCE LISTING (163 sequences)

SEQ ID NO: 1 - protein, 21 aa, Artificial
  zinc finger
XSXXLXXXXXXLSXXXXXRXX

SEQ ID NO: 2 - DNA, 174 bp, Artificial
  HIV-1 LTR
agctttctacaagggactttccgctggggactttccagggaggcgtggcctgggcgggactggggagtggcgtccctcagatgctgcatataagcagctgctttttgcctgtactgggtctctctggttagaccagatctgagcctgggagctctctggctaactagggaaccc

SEQ ID NO: 3 - DNA, 13 bp, Artificial
  octamer-GARAT
atgctaatgarat

SEQ ID NO: 4 - protein, 31 aa, Artificial
  preferred zinc finger framework Formula A
XXCXXXXXCXXXXXXXXXXXXXXHXXXXXXX

SEQ ID NO: 5 - protein, 31 aa, Artificial; identical to SEQ ID NO: 4
  preferred zinc finger framework formula A′

SEQ ID NO: 6 - protein, 24 aa, Artificial
  preferred zinc finger framework Formula B
XCXXXXCXXXFXXXXXLXXHXXXH

SEQ ID NO: 7 - protein, 25 aa, Artificial
  zinc finger consensus structure
PYKCPECGKSFSQKSDLVKHQRTHT

SEQ ID NO: 8 - protein, 25 aa, Artificial
  zinc finger consensus structure
PYKCSECGKAFSQKSNLTRHQRIHT

SEQ ID NO: 9 - protein, 4 aa, Artificial
  canonical linker
GERP

SEQ ID NO: 10 - protein, 4 aa, Artificial
  canonical linker
GEKP

SEQ ID NO: 11 - protein, 4 aa, Artificial
  canonical linker
GQRP

SEQ ID NO: 12 - protein, 4 aa, Artificial
  canonical linker
GQKP

SEQ ID NO: 13 - protein, 5 aa, Artificial
  linker
GGEKP

SEQ ID NO: 14 - protein, 5 aa, Artificial
  linker
GGQKP

SEQ ID NO: 15 - protein, 7 aa, Artificial
  linker
GGSGEKP

SEQ ID NO: 16 - protein, 7 aa, Artificial
  linker
GGSGQKP

SEQ ID NO: 17 - protein, 10 aa, Artificial
  linker
GGSGGSGEKP

SEQ ID NO: 18 - protein, 10 aa, Artificial
  linker
GGSGGSGQKP

SEQ ID NO: 19 - protein, 31 aa, Artificial
  HIV-A F1
XXCXXXXXCXXXXXXXRSDELTRHXXXXXXX

SEQ ID NO: 20 - protein, 31 aa, Artificial
  HIV-A F2
XXCXXXXXCXXXXXXXRSDNLSTHXXXXXXX

SEQ ID NO: 21 - protein, 31 aa, Artificial
  HIV-A F3
XXCXXXXXCXXXXXXXRRDHRTTHXXXXXXX

SEQ ID NO: 22 - protein, 31 aa, Artificial
  HIV-A′ F1
XXCXXXXXCXXXXXXXRSDVLTRHXXXXXXX

SEQ ID NO: 23 - protein, 31 aa, Artificial
  HIV-A′ F2
XXCXXXXXCXXXXXXXRSDHLTTHXXXXXXX

SEQ ID NO: 24 - protein, 31 aa, Artificial
  HIV-A′ F3
XXCXXXXXCXXXXXXXDYSVRKRHXXXXXXX

SEQ ID NO: 25 - protein, 31 aa, Artificial
  HIV-B F1
XXCXXXXXCXXXXXXXDSAHLTRHXXXXXXX

SEQ ID NO: 26 - protein, 31 aa, Artificial
  HIV-B F2
XXCXXXXXCXXXXXXXRSDHLSTHXXXXXXX

SEQ ID NO: 27 - protein, 31 aa, Artificial
  HIV-B F3
XXCXXXXXCXXXXXXXDSANRTKHXXXXXXX

SEQ ID NO: 28 - protein, 31 aa, Artificial
  HIV-C F1
XXCXXXXXCXXXXXXXASADLTRHXXXXXXX

SEQ ID NO: 29 - protein, 31 aa, Artificial
  HIV-C F2
XXCXXXXXCXXXXXXXNRSDLSRHXXXXXXX

SEQ ID NO: 30 - protein, 31 aa, Artificial
  HIV-C F3
XXCXXXXXCXXXXXXXTSSNRKKHXXXXXXX

SEQ ID NO: 31 - protein, 31 aa, Artificial
  HIV-D F1
XXCXXXXXCXXXXXXXHSSDLTRHXXXXXXX

SEQ ID NO: 32 - protein, 31 aa, Artificial
  HIV-D F2
XXCXXXXXCXXXXXXXQSSDLSKHXXXXXXX

SEQ ID NO: 33 - protein, 31 aa, Artificial
  HIV-D F3
XXCXXXXXCXXXXXXXQNATRKRHXXXXXXX

SEQ ID NO: 34 - protein, 31 aa, Artificial
  HIV-E F1
XXCXXXXXCXXXXXXXDSSSLTKHXXXXXXX

SEQ ID NO: 35 - protein, 31 aa, Artificial
  HIV-E F2
XXCXXXXXCXXXXXXXQSAHLSTHXXXXXXX

SEQ ID NO: 36 - protein, 31 aa, Artificial
  HIV-E F3
XXCXXXXXCXXXXXXXDSSSRTKHXXXXXXX

SEQ ID NO: 37 - protein, 31 aa, Artificial
  HIV-F F1
XXCXXXXXCXXXXXXXASDDLTQHXXXXXXX

SEQ ID NO: 38 - protein, 31 aa, Artificial
  HIV-F F2
XXCXXXXXCXXXXXXXRSSDLSRHXXXXXXX

SEQ ID NO: 39 - protein, 31 aa, Artificial
  HIV-F F3
XXCXXXXXCXXXXXXXQSAHRTKHXXXXXXX

SEQ ID NO: 40 - protein, 31 aa, Artificial
  HIV-G F1
XXCXXXXXCXXXXXXXRSDALIQHXXXXXXX

SEQ ID NO: 41 - protein, 31 aa, Artificial
  HIV-G F2
XXCXXXXXCXXXXXXXDRANLSTHXXXXXXX

SEQ ID NO: 42 - protein, 31 aa, Artificial
  HIV-G F3
XXCXXXXXCXXXXXXXASSTRTKHXXXXXXX

SEQ ID NO: 43 - protein, 95 aa, Artificial
  HIV-A
XXCXXXXXCXXXXXXXRSDELTRHXXXXXXXXXXCXXXXXCXXXXXXXRSDNLSTHXXXXXXXXXXCXXXXXCXXXXXXXRRDHRTTHXXXXXXX

SEQ ID NO: 44 - protein, 95 aa, Artificial
  HIV-A′
XXCXXXXXCXXXXXXXDSAHLTRHXXXXXXXXXXCXXXXXCXXXXXXXRSDHLSTHXXXXXXXXXXCXXXXXCXXXXXXXDSANRTKHXXXXXXX

SEQ ID NO: 45 - protein, 95 aa, Artificial
  HIV-B
XXCXXXXXCXXXXXXXRSDVLTRHXXXXXXXXXXCXXXXXCXXXXXXXRSDHLTTHXXXXXXXXXXCXXXXXCXXXXXXXDYSVRKRHXXXXXXX

SEQ ID NO: 46 - protein, 179 aa, Artificial
  HIV-A′A
MAERPYACPVESCDRRFSRSDVLTRHIRIHTGQKPFQCRICMRNFSRSDHLTTHIRTHTGEKPFACDICGRKFADYSVRKRHTKIHTGGSGGSGERPYACPVESCDRRFSRSDELTRHIRIHTGQKPFQCRICMRNFSRSDNLSTHIRTHTGEKPFACDICGRKFARRDHRTTHTKIHL

SEQ ID NO: 47 - protein, 193 aa, Artificial
  HIV-BA
MAERPYACPVESCDRRFSDSAHLTRHIRIHTGQKPFQCRICMRNFSRSDHLSTHIRTHTGEKPFACDICGRKFADSANRTKHTKIHLRQKDGGSGGSGGSGGSGGSGGSERPYACPVESCDRRFSRSDELTRHIRIHTGQKPFQCRICMRNFSRSDNLSTHIRTHTGEKPFACDICGRKFARRDHRTTHTKIH

SEQ ID NO: 48 - protein, 175 aa, Artificial
  HIV-BA′
MAERPYACPVESCDRRFSDSAHLTRHIRIHTGQKPFQCRICMRNFSRSDHLSTHIRTHTGEKPFACDICGRKFADSANRTKHTKIHTGGSGERPYACPVESCDRRFSRSDVLTRHIRIHTGQKPFQCRICMRNFSRSDHLTTHIRTHTGEKPFACDICGRKFADYSVRKRHTKIH

SEQ ID NO: 49 - protein, 327 aa, Artificial
  HIV-A′A-KOX
MAERPYACPVESCDRRFSRSDVLTRHIRIHTGQKPFQCRICMRNFSRSDHLTTHIRTHTGEKPFACDICGRKFADYSVRKRHTKIHTGGSGGSGERPYACPVESCDRRFSRSDELTRHIRIHTGQKPFQCRICMRNFSRSDNLSTHIRTHTGEKPFACDICGRKFARRDHRTTHTKIHLRQKDAARNSGPKKKRKVDGGGALSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVEQKLISEEDL

SEQ ID NO: 50 - protein, 342 aa, Artificial
  HIV-BA-KOX
MAERPYACPVESCDRRFSDSAHLTRHIRIHTGQKPFQCRICMRNFSRSDHLSTHIRTHTGEKPFACDICGRKFADSANRTKHTKIHLRQKDGGSGGSGGSGGSGGSGGSERPYACPVESCDRRFSRSDELTRHIRIHTGQKPFQCRICMRNFSRSDNLSTHIRTHTGEKPFACDICGRKFARRDHRTTHTKIHLRQKDAARNSGPKKKRKVDGGGALSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVEQKLISEEDL

SEQ ID NO: 51 - protein, 324 aa, Artificial
  HIV-BA′-KOX
MAERPYACPVESCDRRFSDSAHLTRHIRIHTGQKPFQCRICMRNFSRSDHLSTHIRTHTGEKPFACDICGRKFADSANRTKHTKIHTGGSGERPYACPVESCDRRFSRSDVLTRHIRIHTGQKPFQCRICMRNFSRSDHLTTHIRTHTGEKPFACDICGRKFADYSVRKRHTKIHLRQKDAARNSGPKKKRKVDGGGALSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVEQKLISEEDL

SEQ ID NO: 52 - protein, 31 aa, Artificial; identical to SEQ ID NO: 19
  4/3 F1

SEQ ID NO: 53 - protein, 31 aa, Artificial; identical to SEQ ID NO: 26
  4/3 F2

SEQ ID NO: 54 - protein, 31 aa, Artificial
  4/3 F3
XXCXXXXXCXXXXXXXTNSNRIKHXXXXXXX

SEQ ID NO: 55 - protein, 31 aa, Artificial; identical to SEQ ID NO: 19
  4A F1

SEQ ID NO: 56 - protein, 31 aa, Artificial
  4A F2
XXCXXXXXCXXXXXXXRSDHLSEHXXXXXXX

SEQ ID NO: 57 - protein, 31 aa, Artificial
  4A F3
XXCXXXXXCXXXXXXXTNNNRKKHXXXXXXX

SEQ ID NO: 58 - protein, 31 aa, Artificial
  7N F1
XXCXXXXXCXXXXXXXTRTNLTRHXXXXXXX

SEQ ID NO: 59 - protein, 31 aa, Artificial
  7N F2
XXCXXXXXCXXXXXXXQDAHLSTHXXXXXXX

SEQ ID NO: 60 - protein, 31 aa, Artificial
  7N F3
XXCXXXXXCXXXXXXXQSANRKTHXXXXXXX

SEQ ID NO: 61 - protein, 95 aa, Artificial
  4/3
XXCXXXXXCXXXXXXXRSDELTRHXXXXXXXXXXCXXXXXCXXXXXXXRSDHLSTHXXXXXXXXXXCXXXXXCXXXXXXXTNSNRIKHXXXXXXX

SEQ ID NO: 62 - protein, 95 aa, Artificial
  4A
XXCXXXXXCXXXXXXXRSDELTRHXXXXXXXXXXCXXXXXCXXXXXXXRSDHLSEHXXXXXXXXXXCXXXXXCXXXXXXXTNNNRKKHXXXXXXX

SEQ ID NO: 63 - protein, 95 aa, Artificial
  7N
XXCXXXXXCXXXXXXXTRTNLTRHXXXXXXXXXXCXXXXXCXXXXXXXQDAHLSTHXXXXXXXXXXCXXXXXCXXXXXXXQSANRKTHXXXXXXX

SEQ ID NO: 64 - protein, 94 aa, Artificial
  4/3
MAEERPYACPVESCDRRFSRSDELTRHIRIHTGQKPFQCRICMRNFSRSDHLSTHIRTHTGEKPFACDICGRKFATNSNRIKHTKIHLRQKDAA

SEQ ID NO: 65 - protein, 94 aa, Artificial
  4A
MAEERPYACPVESCDRRFSRSDELTRHIRIHTGQKPFQCRICMRNFSRSDHLSEHIRTHTGEKPFACDICGRKFATNNNRKKHTKIHLRQKDAA

SEQ ID NO: 66 - protein, 94 aa, Artificial
  7N
MAEERPYACPVESCDRRFSTRTNLTRHIRIHTGQKPFQCRICMRNFSQDAHLSTHIRTHTGEKPFACDICGRKFAQSANRKTHTKIHLRQKDAA

SEQ ID NO: 67 - protein, 191 aa, Artificial
  6F6
MAEERPYACPVESCDRRFSTRTNLTRHIRIHTGQKPFQCRICMRNFSQDAHLSTHIRTHTGEKPFACDICGRKFAQSANRKTHTKIHLRQKDGERPYACPVESCDRRFSRSDELTRHIRIHTGQKPFQCRICMRNFSRSDHLSTHIRTHTGEKPFACDICGRKFATNSNRIKHTKIHLRQKDAARNSTTLD

SEQ ID NO: 68 - protein, 324 aa, Artificial
  6F6 KOX
MAEERPYACPVESCDRRFSTRTNLTRHIRIHTGQKPFQCRICMRNFSQDAHLSTHIRTHTGEKPFACDICGRKFAQSANRKTHTKIHLRQKDGERPYACPVESCDRRFSRSDELTRHIRIHTGQKPFQCRICMRNFSRSDHLSTHIRTHTGEKPFACDICGRKFATNSNRIKHTKIHLRQKDAARNSGPKKRKVDGGGALSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVEQKLISEDL

SEQ ID NO: 69 - DNA, 10 bp, Artificial
  NF-kB
gggaaattcc

SEQ ID NO: 70 - DNA, 10 bp, Artificial
  Sp1
ngggcggnnn

SEQ ID NO: 71 - DNA, 98 bp, Artificial
  Sfi Val3
gcaactgcggcccagccggccatggcagaggaacgcccatatgcttgccctgtcgagtcctgcgatcgccgcttttctcgctcggatgtccttacccg

SEQ ID NO: 72 - DNA, 84 bp, Artificial
  NotGCC
gagtcattctgcggccgcgtccttctgtcttaaatggattttggtatgcctcttgcgcdmgctgkrgtsggcaaacttcctccc

SEQ ID NO: 73 - DNA, 10 bp, Artificial
  HIV-A′ DNA target site
gcctgggcgg

SEQ ID NO: 74 - DNA, 10 bp, Artificial
  HIV-A DNA target site
agggaggcgt

SEQ ID NO: 75 - DNA, 10 bp, Artificial
  HIV-B DNA target site
gacggtggag

SEQ ID NO: 76 - DNA, 10 bp, Artificial
  HIV-C DNA target site
gatgctgcat

SEQ ID NO: 77 - DNA, 10 bp, Artificial
  HIV-D DNA target site
gcagctgctt

SEQ ID NO: 78 - DNA, 10 bp, Artificial
  HIV-E DNA target site
atctgagcct

SEQ ID NO: 79 - DNA, 10 bp, Artificial
  HIV-F DNA target site
ggagctctct

SEQ ID NO: 80 - DNA, 10 bp, Artificial
  HIV-G DNA target site
gctaactagg

SEQ ID NO: 81 - protein, 21 aa, Artificial
  HIV-A zinc finger
RSDELTRRSDNLSTRRDHRTT

SEQ ID NO: 82 - protein, 21 aa, Artificial
  HIV-A′ zinc finger
RSDVLTRRSDHLTTDYSVRKR

SEQ ID NO: 83 - protein, 21 aa, Artificial
  HIV-B zinc finger
DSAHLTRRSDHLSTDSANRTK

SEQ ID NO: 84 - protein, 21 aa, Artificial
  HIV-C zinc finger
ASADLTRNRSDLSRTSSNRKK

SEQ ID NO: 85 - protein, 21 aa, Artificial
  HIV-D zinc finger
HSSDLTRQSSDLSKQNATRKR

SEQ ID NO: 86 - protein, 21 aa, Artificial
  HIV-E zinc finger
DSSSLTKQSAHLSTDSSSRTK

SEQ ID NO: 87 - protein, 21 aa, Artificial
  HIV-F zinc finger
ASDDLTQRSSDLSRQSAHRTK

SEQ ID NO: 88 - protein, 21 aa, Artificial
  HIV-G zinc finger
RSDALIQDRANLSTASSTRTK

SEQ ID NO: 89 - protein, 91 aa, Artificial
  HIV-A sequence
MAERPYACPVESCDRRFSRSDELTRHIRIHTGQKPFQCRICMRNFSRSDNLSTHIRTHTGEKPFACDICGRKFARRDHRTTHTKIHLRQKD

SEQ ID NO: 90 - protein, 91 aa, Artificial
  HIV-A′ sequence
MAERPYACPVESCDRRFSRSDVLTRHIRIHTGQKPFQCRICMRNFSRSDHLTTHIRTHTGEKPFACDICGRKFADYSVRKRHTKIHLRQKD

SEQ ID NO: 91 - protein, 91 aa, Artificial
  HIV-B sequence
MAERPYACPVESCDRRFSDSAHLTRHIRIHTGQKPFQCRICMRNFSRSDHLSTHIRTHTGEKPFACDICGRKFADSANRTKHTKIHLRQKD

SEQ ID NO: 92 - protein, 11 aa, Artificial
  HIV-A′ and HIV-A linker
TGGSGGSGERP

SEQ ID NO: 93 - protein, 183 aa, Artificial
  HIV-A′A sequence
MAERPYACPVESCDRRFSRSDVLTRHIRIHTGQKPFQCRICMRNFSRSDHLTTHIRTHTGEKPFACDICGRKFADYSVRKRHTKIHTGGSGGSGERPYACPVESCDRRFSRSDELTRHIRIHTGQKPFQCRICMRNFSRSDNLSTHIRTHTGEKPFACDICGRKFARRDHRTTHTKIHLRQKD

SEQ ID NO: 94 - protein, 26 aa, Artificial
  HIV-B and HIV-A linker
LRQKDGGSGGSGGSGGSGGSGGSERP

SEQ ID NO: 95 - protein, 198 aa, Artificial
  HIV-BA sequence
MAERPYACPVESCDRRFSDSAHLTRHIRIHTGQKPFQCRICMRNFSRSDHLSTHIRTHTGEKPFACDICGRKFADSANRTKHTKIHLRQKDGGSGGSGGSGGSGGSGGSERPYACPVESCDRRFSRSDELTRHIRIHTGQKPFQCRICMRNFSRSDNLSTHIRTHTGEKPFACDICGRKFARRDHRTTHTKIHLRQKD

SEQ ID NO: 96 - protein, 8 aa, Artificial
  HIV-B and HIV-A′ linker
TGGSGERP

SEQ ID NO: 97 - protein, 180 aa, Artificial
  HIV-BA′ sequence
MAERPYACPVESCDRRFSDSAHLTRHIRIHTGQKPFQCRICMRNFSRSDHLSTHIRTHTGEKPFACDICGRKFADSANRTKHTKIHTGGSGERPYACPVESCDRRFSRSDVLTRHIRIHTGQKPFQCRICMRNFSRSDHLTTHIRTHTGEKPFACDICGRKFADYSVRKRHTKIHLRQKD

SEQ ID NO: 98 - protein, 144 aa, Artificial
  NLS-KOX1-c-myc domain sequence
AARNSGPKKKRKVDGGGALSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVEQKLISEEDL

SEQ ID NO: 99 - DNA, 708 bp, Artificial
  HIV A-KOX sequence
atggcagagcggccgtatgcttgccctgtcgagtcctgcgatcgccgcttttctcgctcggatgagcttacccgccatatccgcatccacacaggccagaagcccttccagtgtcgaatctgcatgcgtaacttcagtcgtagtgacaacctgagcacgcacatccgcacccacacaggcgagaagccttttgcctgtgacatttgtgggaggaaatttgcccggagggaccaccgcacaacgcataccaagatacacctgcgccaaaaagatgcggcccggaattccggcccaaaaaagaagagaaaggtcgacggcggtggtgctttgtctcctcagcactctgctgtcactcaaggaagtatcatcaagaacaaggagggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttgaacaaaaacttatttctgaagaagatctgtaa

SEQ ID NO: 100 - protein, 235 aa, Artificial
  HIV A-KOX sequence
MAERPYACPVESCDRRFSRSDELTRHIRIHTGQKPFQCRICMRNFSRSDNLSTHIRTHTGEKPFACDICGRKFARRDHRTTHTKIHLRQKDAARNSGPKKKRKVDGGGALSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVEQKLISEEDL

SEQ ID NO: 101 - DNA, 708 bp, Artificial
  HIV A′-KOX sequence
atggcagaacgcccgtatgcttgccctgtcgagtcctgcgatcgccgcttttctcgctcggatgtccttacccgccatatccgcatccacacaggccagaagcccttccagtgtcgaatctgcatgcgtaacttcagtcgtagtgaccaccttaccacccacatccgcacccacacaggcgagaagccttttgcctgtgacatttgtgggaggaagtttgccgactacagcgtacgcaagaggcataccaaaatccatctgcgccaaaaagatgcggcccggaattccggcccaaaaaagaagagaaaggtcgacggcggtggtgctttgtctcctcagcactctgctgtcactcaaggaagtatcatcaagaacaaggagggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttgaacaaaaacttatttctgaagaagatctgtaa

SEQ ID NO: 102 - protein, 235 aa, Artificial
  HIV A′-KOX sequence
MAERPYACPVESCDRRFSRSDVLTRHIRIHTGQKPFQCRICMRNFSRSDHLTTHIRTHTGEKPFACDICGRKFADYSVRKRHTKIHLRQKDAARNSGPKKKRKVDGGGALSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVEQKLISEEDL

SEQ ID NO: 103 - DNA, 708 bp, Artificial
  HIV B-KOX sequence
atggcggagaggccctacgcatgccctgtcgagtcctgcgatcgccgcttttctgactcggcccaccttacccggcatatccgcatccacaccggtcagaagcccttccagtgtcgaatctgcatgcgtaacttcagtcggagcgaccacctgagcacccacatccgcacccacacaggcgagaagccttttgcctgtgacatttgtgggaggaaatttgccgacagcgccaaccgcacaaagcataccaagatacacctgcgccaaaaagatgcggcccggaattccggcccaaaaaagaagagaaaggtcgacggcggtggtgctttgtctcctcagcactctgctgtcactcaaggaagtatcatcaagaacaaggagggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttgaacaaaaacttatttctgaagaagatctgtaa

SEQ ID NO: 104 - protein, 235 aa, Artificial
  HIV B-KOX sequence
MAERPYACPVESCDRRFSDSAHLTRHIRIHTGQKPFQCRICMRNFSRSDHLSTHIRTHTGEKPFACDICGRKFADSANRTKHTKIHLRQKDAARNSGPKKKRKVDGGGALSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVEQKLISEEDL

SEQ ID NO: 105 - DNA, 984 bp, Artificial
  HIV A′A-KOX sequence
atggcagaacgcccgtatgcttgccctgtcgagtcctgcgatcgccgcttttctcgctcggatgtccttacccgccatatccgcatccacacaggccagaagcccttccagtgtcgaatctgcatgcgtaacttcagtcgtagtgaccaccttaccacccacatccgcacccacacaggcgagaagccttttgcctgtgacatttgtgggaggaagtttgccgactacagcgtacgcaagaggcataccaaaatccataccggcgggagcggcgggagcggcgagcggccgtatgcttgccctgtcgagtcctgcgatcgccgcttttctcgctcggatgagcttacccgccatatccgcatccacacaggccagaagcccttccagtgtcgaatctgcatgcgtaacttcagtcgtagtgacaacctgagcacgcacatccgcacccacacaggcgagaagccttttgcctgtgacatttgtgggaggaaatttgcccggagggaccaccgcacaacgcataccaagatacacctgcgccaaaaagatgcggcccggaattccggcccaaaaaagaagagaaaggtcgacggcggtggtgctttgtctcctcagcactctgctgtcactcaaggaagtatcatcaagaacaaggagggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttgaacaaaaacttatttctgaagaagatctgtaa

SEQ ID NO: 106 - protein, 327 aa, Artificial; identical to SEQ ID NO: 49
  HIV A′A-KOX sequence

SEQ ID NO: 107 - DNA, 1029 bp, Artificial
  HIV BA-KOX sequence
atggcggagaggccctacgcatgccctgtcgagtcctgcgatcgccgcttttctgactcggcccaccttacccggcatatccgcatccacaccggtcagaagcccttccagtgtcgaatctgcatgcgtaacttcagtcggagcgaccacctgagcacccacatccgcacccacacaggcgagaagccttttgcctgtgacatttgtgggaggaaatttgccgacagcgccaaccgcacaaagcataccaagatacacctgcgccaaaaagatgggggcagcggcgggtccggggggagcggcggctccgggggcagcggcgggtccgagcggccgtatgcttgccctgtcgagtcctgcgatcgccgcttttctcgctcggatgagcttacccgccatatccgcatccacacaggccagaagcccttccagtgtcgaatctgcatgcgtaacttcagtcgtagtgacaacctgagcacgcacatccgcacccacacaggcgagaagccttttgcctgtgacatttgtgggaggaaatttgcccggagggaccaccgcacaacgcataccaagatacacctgcgccaaaaagatgcggcccggaattccggcccaaaaaagaagagaaaggtcgacggcggtggtgctttgtctcctcagcactctgctgtcactcaaggaagtatcatcaagaacaaggagggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttgaacaaaaacttatttctgaagaagatctgtaa

SEQ ID NO: 108 - protein, 342 aa, Artificial; identical to SEQ ID NO: 50
  HIV BA-KOX sequence

SEQ ID NO: 109 - DNA, 975 bp, Artificial
  HIV BA′-KOX sequence
atggcggagaggccctacgcatgccctgtcgagtcctgcgatcgccgcttttctgactcggcccaccttacccggcatatccgcatccacaccggtcagaagcccttccagtgtcgaatctgcatgcgtaacttcagtcggagcgaccacctgagcacccacatccgcacccacacaggcgagaagccttttgcctgtgacatttgtgggaggaaatttgccgacagcgccaaccgcacaaagcataccaagatacacaccggcgggagcggcgagcggccgtatgcttgccctgtcgagtcctgcgatcgccgcttttctcgctcggatgtccttacccgccatatccgcatccacacaggccagaagcccttccagtgtcgaatctgcatgcgtaacttcagtcgtagtgaccaccttaccacccacatccgcacccacacaggcgagaagccttttgcctgtgacatttgtgggaggaagtttgccgactacagcgtgcgcaagaggcataccaaaatccatttaagacagaaggacgcggcccggaattccggcccaaaaaagaagagaaaggtcgacggcggtggtgctttgtctcctcagcactctgctgtcactcaaggaagtatcatcaagaacaaggagggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttgaacaaaaacttatttctgaagaagatctgtaa

SEQ ID NO: 110 - protein, 324 aa, Artificial; identical to SEQ ID NO: 51
  HIV BA′-KOX sequence

SEQ ID NO: 111 - DNA, 25 bp, Artificial
  HSV IE175K
gatcgggcggtaatgagatgccatg

SEQ ID NO: 112 - DNA, 282 bp, Artificial
  clone 4/3 sequence
atggcagaggaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccacctgagcacgcacatccgcacccacacaggcgagaagccttttgcctgtgacatttgtgggaggaaatttgccaccaacagcaaccgcataaagcataccaagatacacctgcgccaaaaagatgcggcc

SEQ ID NO: 113 - protein, 94 aa, Artificial; identical to SEQ ID NO: 64
  clone 4/3 sequence

SEQ ID NO: 114 - DNA, 282 bp, Artificial
  clone 4A sequence
atggcagaggaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccacctgagcgagcacatccgcacccacacaggcgagaagccttttgcctgtgacatttgtgggaggaaatttgccaccaacaacaaccgcaaaaagcataccaagatacacctgcgccaaaaagatgcggcc

SEQ ID NO: 115 - protein, 94 aa, Artificial; identical to SEQ ID NO: 65
  clone 4A sequence

SEQ ID NO: 116 - DNA, 282 bp, Artificial
  clone 7N sequence
atggcagaggaacgcccatatgcttgccctgtcgagtcctgcgatcgccgcttttctacgcgaactaaccttacccgccatatccgcatccacacaggccagaagcccttccagtgtcgaatctgcatgcgtaacttcagtcaggacgcacacctgagcacgcacatccgcacccacacaggcgagaagccttttgcctgtgacatttgtgggaggaaatttgcccagagcgccaaccgcaaaacgcataccaagatacacctgcgccaaaaagatgcggcc

SEQ ID NO: 117 - protein, 94 aa, Artificial; identical to SEQ ID NO: 66
  clone 7N sequence

SEQ ID NO: 118 - DNA, 576 bp, Artificial
  clone 6F6 sequence
atggcagaggaacgcccatatgcttgccctgtcgagtcctgcgatcgccgcttttctacgcgaactaaccttacccgccatatccgcatccacacaggccagaagcccttccagtgtcgaatctgcatgcgtaacttcagtcaggacgcacacctgagcacgcacatccgcacccacacaggcgagaagccttttgcctgtgacatttgtgggaggaaatttgcccagagcgccaaccgcaaaacgcataccaagatacacctgcgccaaaaagatggcgaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccacctgagcacgcacatccgcacccacacaggcgagaagccttttgcctgtgacatttgtgggaggaaatttgccaccaacagcaaccgcataaagcataccaagatacacctgcgccaaaaagatgcggcccggaattccaccacactggactag

SEQ ID NO: 119 - protein, 191 aa, Artificial; identical to SEQ ID NO: 67
  clone 6F6 sequence

SEQ ID NO: 120 - DNA, 975 bp, Artificial
  6F6-KOX sequence
atggcagaggaacgcccatatgcttgccctgtcgagtcctgcgatcgccgcttttctacgcgaactaaccttacccgccatatccgcatccacacaggccagaagcccttccagtgtcgaatctgcatgcgtaacttcagtcaggacgcacacctgagcacgcacatccgcacccacacaggcgagaagccttttgcctgtgacatttgtgggaggaaatttgcccagagcgccaaccgcaaaacgcataccaagatacacctgcgccaaaaagatggcgaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccacctgagcacgcacatccgcacccacacaggcgagaagccttttgcctgtgacatttgtgggaggaaatttgccaccaacagcaaccgcataaagcataccaagatacacctgcgccaaaaagatgcggcccggaattccggcccaaaaaagagaaaggtcgacggcggtggtgctttgtctcctcagcactctgctgtcactcaaggaagtatcatcaagaacaaggagggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttgaacaaaaacttatttctgaagatctgtaa

SEQ ID NO: 121 - protein, 324 aa, Artificial; identical to SEQ ID NO: 68
  clone 6F6-KOX sequence

SEQ ID NO: 122 - DNA, 33 bp, Artificial
  4AFOR primer
ctgctctagagcgccgccatggcagaggaacgc

SEQ ID NO: 123 - DNA, 46 bp, Artificial
  HIV13Rev primer
tccgggatcccgcggaattccgggccgcatctttttggcgcaggtg

SEQ ID NO: 124 - DNA, 33 bp, Artificial
  HIV13For primer
ctctagagcgccgccatggcggaagagaggccc

SEQ ID NO: 125 - DNA, 25 bp, Artificial
  NCFUS2 primer
gaaacgcccatatgcttgccctgtc

SEQ ID NO: 126 - DNA, 51 bp, Artificial
  RevlinGly primer
cagggcaagcatatgggcgttcgccatctttttggcgcaggtgtatcttgg

SEQ ID NO: 127 - DNA, 44 bp, Artificial
  FOR2 primer
gacagaaggacgcggccacgcgtccaaaaaagaagagaaaggtc

SEQ ID NO: 128 - DNA, 66 bp, Artificial
  REV2 primer
cgcggatccttacagatcttcttcagaaataagtttttgttcaactgatgatttgatttcaaatgc

SEQ ID NO: 129 - DNA, 34 bp, Artificial
  6F6HIND FOR primer
ctacgtaagcttgcgccgccatggcagaggaacg

SEQ ID NO: 130 - DNA, 28 bp, Artificial
  KOX/VP16REV
gctcggatccttacagatcttcttcaga

SEQ ID NO: 131 - DNA, 31 bp, Artificial
  T24 probe
ccgccggatcgggcggtaatgagatgccatg

SEQ ID NO: 132 - DNA, 30 bp, Artificial
  H2B probe
atagaatcgcttatgcaaataaggtgaaga

SEQ ID NO: 133 - DNA, 30 bp, Artificial
  68K probe
cttcccggttcggcggtaatgagatacgag

SEQ ID NO: 134 - DNA, 30 bp, Artificial
  IE110 probe
tgggttccgggtatggtaatgagtttcttc

SEQ ID NO: 135 - protein, 7 aa, Artificial
  linker
QKDGERP

SEQ ID NO: 136 - protein, 7 aa, Artificial
  zinc finger motif
RSDELTR

SEQ ID NO: 137 - protein, 7 aa, Artificial
  zinc finger motif
RSDNLST

SEQ ID NO: 138 - protein, 7 aa, Artificial
  zinc finger motif
RRDHRTT

SEQ ID NO: 139 - protein, 7 aa, Artificial
  zinc finger motif
RSDVLTR

SEQ ID NO: 140 - protein, 7 aa, Artificial
  zinc finger motif
RSDHLTT

SEQ ID NO: 141 - protein, 7 aa, Artificial
  zinc finger motif
DYSVRKR

SEQ ID NO: 142 - protein, 7 aa, Artificial
  zinc finger motif
DSAHLTR

SEQ ID NO: 143 - protein, 7 aa, Artificial
  zinc finger motif
RSDHLST

SEQ ID NO: 144 - protein, 7 aa, Artificial
  zinc finger motif
DSANRTK

SEQ ID NO: 145 - protein, 7 aa, Artificial
  zinc finger motif
ASADLTR

SEQ ID NO: 146 - protein, 7 aa, Artificial
  zinc finger motif
NRSDLSR

SEQ ID NO: 147 - protein, 7 aa, Artificial
  zinc finger motif
TSSNRKK

SEQ ID NO: 148 - protein, 7 aa, Artificial
  zinc finger motif
HSSDLTR

SEQ ID NO: 149 - protein, 7 aa, Artificial
  zinc finger motif
QSSDLSK

SEQ ID NO: 150 - protein, 7 aa, Artificial
  zinc finger motif
QNATRKR

SEQ ID NO: 151 - protein, 7 aa, Artificial
  zinc finger motif
DSSSLTK

SEQ ID NO: 152 - protein, 7 aa, Artificial
  zinc finger motif
QSAHLST

SEQ ID NO: 153 - protein, 7 aa, Artificial
  zinc finger motif
DSSSRTK

SEQ ID NO: 154 - protein, 7 aa, Artificial
  zinc finger motif
ASDDLTQ

SEQ ID NO: 155 - protein, 7 aa, Artificial
  zinc finger motif
RSSDLSR

SEQ ID NO: 156 - protein, 7 aa, Artificial
  zinc finger motif
QSAHRTK

SEQ ID NO: 157 - protein, 7 aa, Artificial
  zinc finger motif
RSDALIQ

SEQ ID NO: 158 - protein, 7 aa, Artificial
  zinc finger motif
DRANLST

SEQ ID NO: 159 - protein, 7 aa, Artificial
  zinc finger motif
ASSTRTK

SEQ ID NO: 160 - protein, 7 aa, Artificial
  zinc finger motif
TNSNRIK

SEQ ID NO: 161 - protein, 7 aa, Artificial
  zinc finger motif
TRTNLTR

SEQ ID NO: 162 - protein, 7 aa, Artificial
  zinc finger motif
QDAHLST

SEQ ID NO: 163 - protein, 7 aa, Artificial
  zinc finger motif
QSANRKT